SEQUENCE LISTING (145 sequences)

SEQ ID NO: 1 - protein, 6 aa, artificial sequence
  Abeta 1-6
DAEFRH

SEQ ID NO: 2 - protein, 7 aa, artificial sequence
  Abeta 1-7
DAEFRHD

SEQ ID NO: 3 - protein, 4 aa, artificial sequence
  Abeta 3-6
EFRH

SEQ ID NO: 4 - protein, 5 aa, artificial sequence
  Abeta 1-5
DAEFR

SEQ ID NO: 5 - protein, 222 aa, artificial sequence
  CMVNtt830
MGQYIKANSKFIGITERRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 6 - protein, 238 aa, artificial sequence
  CMV-Ntt830-Ab16
MGQYIKANSKFIGITERRRRPRRGSRSAPSSADANFRVLSQQLSRLNKTLAAGRPTINHPTFVGSERCKPGYTFTSITLKPPKIDRGSGGGSDAEFRHGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 7 - protein, 239 aa, artificial sequence
  CMV-Ntt830-Ab17
MGQYIKANSKFIGITERRRRPRRGSRSAPSSADANFRVLSQQLSRLNKTLAAGRPTINHPTFVGSERCKPGYTFTSITLKPPKIDRGSGGGSDAEFRHDGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 8 - protein, 244 aa, artificial sequence
  CMV-Ntt830-Ab36
CMVNTTABMGQYIKANSKFIGITERRRRPRRGSRSAPSSADANFRVLSQQLSRLNKTLAAGRPTINHPTFVGSERCKPGYTFTSITLKPPKIDRGSGGGSEFRHGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 9 - protein, 237 aa, artificial sequence
  CMV-Ntt830-Ab15
MGQYIKANSKFIGITERRRRPRRGSRSAPSSADANFRVLSQQLSRLNKTLAAGRPTINHPTFVGSERCKPGYTFTSITLKPPKIDRGSGGGSDAEFRGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 10 - protein, 4 aa, artificial sequence
  GGGS-linker
GGGS

SEQ ID NO: 11 - protein, 6 aa, artificial sequence
  GGGSGS-linker
GGGSGS

SEQ ID NO: 12 - DNA, 726 bp, artificial sequence
  CMV-Ntt830-Ab16
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgatgcggagtttcgccatgggggtggttcgggttcctattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 13 - DNA, 729 bp, artificial sequence
  CMV-Ntt830-Ab17
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgatgcggagtttcgccatgatgggggtggttcgggttcctattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 14 - DNA, 720 bp, artificial sequence
  CMV-Ntt830-Ab36
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgagtttcgccatgggggtggttcgggttcctattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 15 - DNA, 723 bp, artificial sequence
  CMV-Ntt830-Ab15
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgatgcggagttccgcgggggtggttcgggatcctattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 16 - DNA, 24 bp, artificial sequence
  Forward - pET-90 primer
aggatcgagatctcgatcccgcga

SEQ ID NO: 17 - DNA, 35 bp, artificial sequence
  Reverse - RGSYrev
accataataggatccacggtctatttttggtggct

SEQ ID NO: 18 - DNA, 40 bp, artificial sequence
  Forward - RGSYdir
agaccgtggatcctattatggtaaaaggttgttattacct

SEQ ID NO: 19 - DNA, 25 bp, artificial sequence
  Reverse - CMV-AgeR
agtaccggtgaggctccgtccgcaa

SEQ ID NO: 20 - DNA, 77 bp, artificial sequence
  Forward - C-Ab15
tggatccggtggcggtagcgatgcggagttccgcgggggtggttcgggttcctattatggtaaaaggttgttattac

SEQ ID NO: 21 - DNA, 37 bp, artificial sequence
  Reverse -  CMcpR
caaagcttatcaaactgggagcaccccagatgtggga

SEQ ID NO: 22 - DNA, 82 bp, artificial sequence
  Forward - C-Ab16
cgtggatccggtggcggtagcgatgcggagtttcgccatgggggtggttcgggttcctattatggtaaaaggttgttattac

SEQ ID NO: 23 - DNA, 85 bp, artificial sequence
  Forward - C-Ab17
cgtggatccggtggcggtagcgatgcggagtttcgccatgatgggggtggttcgggttcctattatggtaaaaggttgttattac

SEQ ID NO: 24 - DNA, 76 bp, artificial sequence
  Forward - C-Ab36
cgtggatccggtggcggtagcgagtttcgccatgggggtggttcgggttcctattatggtaaaaggttgttattac

SEQ ID NO: 25 - DNA, 34 bp, artificial sequence
  Forward - CM-830NdeF
acacatatgggccagtatattaaggccaactcca

SEQ ID NO: 26 - DNA, 37 bp, artificial sequence; identical to SEQ ID NO: 21
  Reverse - CM-cpR

SEQ ID NO: 27 - protein, 151 aa, artificial sequence
  Ara-h202
RQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 28 - DNA, 501 bp, artificial sequence
  Ara-h202
atgggctgccgccatcatcatcatcatcatggcgcgcgccagcgccagcagtgggaactgcagggcgatcgtcgctgccagagccagctggaacgcgcgaacctgcgtccgtgcgaacagcatctgatgcagaaaattcagcgcgatgaagatagctatggccgcgatccgtatagcccaagccaagatccgtatagcccaagccaggatccggatcgccgtgatccgtatagcccaagcccgtatgatcgtcgcggcgcgggcagcagccagcatcaggaacgctgctgcaacgaactgaacgaatttgaaaacaaccagcgctgcatgtgcgaagcgctgcaacagattatggaaaaccagagcgatcgcctgcaaggccgccagcaagaacagcagtttaaacgcgaactgcgtaacctgccgcagcagtgcggcctgcgtgcgccgcagcgctgcgatctggaagtggaaagcggcggccgtgatcgctattaataa

SEQ ID NO: 29 - protein, 398 aa, artificial sequence
  CMV-Ntt830-Arah202
MGQYIKANSKFIGITERRRRPRRGSRSAPSSADANFRVLSQQLSRLNKTLAAGRPTINHPTFVGSERCKPGYTFTSITLKPPKIDRGSGGGGSGGGGSGGGGSRQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRYGGGGSGGGGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 30 - protein, 15 aa, artificial sequence
  15aa GS-linker
GGGGSGGGGSGGGGS

SEQ ID NO: 31 - protein, 10 aa, artificial sequence
  10aa GS-linker
GGGGSGGGGS

SEQ ID NO: 32 - DNA, 1215 bp, artificial sequence
  CMV-Ntt830-Arah202
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatctggaggaggtggtagtggtggaggtggatctggaggaggtggatccggtcgccagcagtgggaactgcagggcgatcgtcgctgccagagccagctggaacgcgcgaacctgcgtccgtgcgaacagcatctgatgcagaaaattcagcgcgatgaagatagctatggccgcgatccgtatagcccaagccaagatccgtatagcccaagccaggatccggatcgccgtgatccgtatagcccaagcccgtatgatcgtcgcggcgcgggcagcagccagcatcaggaacgctgctgcaacgaactgaacgaatttgaaaacaaccagcgctgcatgtgcgaagcgctgcaacagattatggaaaaccagagcgatcgcctgcaaggccgccagcaagaacagcagtttaaacgcgaactgcgtaacctgccgcagcagtgcggcctgcgtgcgccgcagcgctgcgatctggaagtggaaagcggcggccgtgatcgctatggatccggaggaggtggaagcggaggaggtggatcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 33 - DNA, 27 bp, artificial sequence
  Forward - 830-NcoF
ataccatgggccagtatattaaggcca

SEQ ID NO: 34 - DNA, 78 bp, artificial sequence
  Reverse - C-5xg4s-R
ggatccacctcctccagatccacctccaccactaccacctcctccagatccacggtctatttttggtggctttagggt

SEQ ID NO: 35 - DNA, 65 bp, artificial sequence
  Forward - C-5xg4s-F
ggatccggaggaggtggaagcggaggaggtggatcttattatggtaaaaggttgttattacctga

SEQ ID NO: 36 - DNA, 32 bp, artificial sequence
  Forward - Ara-BamF2
acaggatccggtcgccagcagtgggaactgca

SEQ ID NO: 37 - DNA, 30 bp, artificial sequence
  Reverse - Ara-BamR2
tgtggatccatagcgatcacggccgccgct

SEQ ID NO: 38 - protein, 177 aa, artificial sequence
  Feld12
EICPAVKRDVDLFLTGTPDEYVEQVAQYKALPVVLENARILKNCVDAKMTEEDKENALSVLDKIYTSPLCGGGGSGGGGSGGGGSVKMAETCPIFYDVFFAVANGNELLLDLSLTKVNATEPERTAMKKIQDCYVENGLISRVLDGLVMTTISSSKDCMGEAVQNTVEDLKLNTLGR

SEQ ID NO: 39 - protein, 424 aa, artificial sequence
  CMV-Ntt830-Feld12
MGQYIKANSKFIGITERRRRPRRGSRSAPSSADANFRVLSQQLSRLNKTLAAGRPTINHPTFVGSERCKPGYTFTSITLKPPKIDRGSGGGGSGGGGSGGGGSEICPAVKRDVDLFLTGTPDEYVEQVAQYKALPVVLENARILKNCVDAKMTEEDKENALSVLDKIYTSPLCGGGGSGGGGSGGGGSVKMAETCPIFYDVFFAVANGNELLLDLSLTKVNATEPERTAMKKIQDCYVENGLISRVLDGLVMTTISSSKDCMGEAVQNTVEDLKLNTLGRGGGGSGGGGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 40 - DNA, 1284 bp, artificial sequence
  CMV-Ntt830-Feld12
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggaggaggtggtagtggtggaggtggatctggaggaggtggaagcgaaatttgtccggcagttaaacgtgatgttgacctgtttctgaccggtacaccggatgaatatgtggaacaggttgcacagtataaagcactgccggttgttctggaaaatgcacgtattctgaaaaattgcgtggatgccaaaatgaccgaagaggataaagaaaatgccctgagcgttctggataaaatctataccagtccgctgtgcggtggtggtggtagtggtggcggtggttcaggcggtggcggtagcgttaaaatggcagaaacctgtccgatcttttatgatgttttttttgccgtggccaatggcaatgaactgctgctggatctgagcctgaccaaagttaatgcaaccgaaccggaacgtaccgcaatgaaaaaaatccaggattgctatgtggaaaacggtctgattagccgtgttctggatggtctggttatgaccaccattagcagcagcaaagattgtatgggtgaagcagtgcagaataccgttgaagatctgaaactgaataccctgggtcgtggaggaggtggaagcggaggaggtggatcctattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 41 - DNA, 77 bp, artificial sequence
  Forward - FG4S-BamF
caggatccggaggaggtggtagtggtggaggtggatctggaggaggtggtagcgaaatttgtccggcagttaaacgt

SEQ ID NO: 42 - DNA, 56 bp, artificial sequence
  Reverse - FG4S-BamR
ataggatccacctcctccgcttccacctcctccacgacccagggtattcagtttca

SEQ ID NO: 43 - protein, 4 aa, artificial sequence
  Plasmodium falciparum CS fragment
NANP

SEQ ID NO: 44 - protein, 76 aa, artificial sequence
  Plasmodium falciparum CS protein fragment
NANPNANPNANPNANPNANPNANPNANPNANPNANPNANPNANPNANPNANPNANPNANPNANPNANPNANPNANP

SEQ ID NO: 45 - DNA, 754 bp, artificial sequence
  Plasmodium falciparum CS protein fragment and CMVNtt830 3′ terminal part
ccctaaagccaccaaaaatagaccgtggatctggaggaggtggtagtggtggaggtggatctggaggaggtggatccaacgcgaatccgaacgcaaacccgaacgcgaatccgaacgcaaacccaaacgcgaatccgaacgcaaacccgaacgcgaacccgaacgcgaacccgaacgcgaacccgaacgcgaaccctaacgcgaacccgaacgcgaacccgaacgcgaacccgaacgcgaacccgaacgcgaacccgaacgcgaacccgaacgcgaacccgaacgcgaacccgaacgcgaacccgggatctggaggaggtggaagcggaggaggtggatcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagcttt

SEQ ID NO: 46 - protein, 325 aa, artificial sequence
  CMV-Ntt830-19NANP
MGQYIKANSKFIGITERRRRPRRGSRSAPSSADANFRVLSQQLSRLNKTLAAGRPTINHPTFVGSERCKPGYTFTSITLKPPKIDRGSGGGGSGGGGSGGGGSNANPNANPNANPNANPNANPNANPNANPNANPNANPNANPNANPNANPNANPNANPNANPNANPNANPNANPNANPGSGGGGSGGGGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 47 - protein, 12 aa, artificial sequence
  12aa GS-linker
GSGGGGSGGGGS

SEQ ID NO: 48 - DNA, 1215 bp, artificial sequence; identical to SEQ ID NO: 32
  CMV-Ntt830-19NANP

SEQ ID NO: 49 - protein, 10 aa, artificial sequence
  alpha-synuclein peptide egy
EGYQDYEPEA

SEQ ID NO: 50 - protein, 8 aa, artificial sequence
  alpha-synuclein petide kne
KNEEGAPQ

SEQ ID NO: 51 - protein, 8 aa, artificial sequence
  alpha-synuclein petide mdv
MDVFMKGL

SEQ ID NO: 52 - protein, 242 aa, artificial sequence
  CMV-Ntt830-egy
MGQYIKANSKFIGITERRRRPRRGSRSAPSSADANFRVLSQQLSRLNKTLAAGRPTINHPTFVGSERCKPGYTFTSITLKPPKIDRGSGGGSEGYQDYEPEAGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 53 - protein, 240 aa, artificial sequence
  CMV-Ntt830-kne
MGQYIKANSKFIGITERRRRPRRGSRSAPSSADANFRVLSQQLSRLNKTLAAGRPTINHPTFVGSERCKPGYTFTSITLKPPKIDRGSGGGSKNEEGAPQGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 54 - protein, 240 aa, artificial sequence
  CMV-Ntt830-mdv
MGQYIKANSKFIGITERRRRPRRGSRSAPSSADANFRVLSQQLSRLNKTLAAGRPTINHPTFVGSERCKPGYTFTSITLKPPKIDRGSGGGSMDVFMKGLGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 55 - DNA, 738 bp, artificial sequence
  CMV-Ntt830-egy
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgaggggtatcaggattatgagccggaggcggggggtggttcgggttcctattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 56 - DNA, 731 bp, artificial sequence
  CMV-Ntt830-kne
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcaagaatgaggagggggcgccgcaggggggtggttcgggttcctattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagct

SEQ ID NO: 57 - DNA, 732 bp, artificial sequence
  CMV-Ntt830-mdv
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcatggatgtgtttatgaaggggttggggggtggttcgggttcctattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 58 - DNA, 71 bp, artificial sequence
  1st PCR Forward CM-egyF
gtggatccggtggcggtagcgaggggtatcaggattatgagccggaggcggggggtggttcgggttcctat

SEQ ID NO: 59 - DNA, 37 bp, artificial sequence; identical to SEQ ID NO: 21
  Reverse CMcpR

SEQ ID NO: 60 - DNA, 65 bp, artificial sequence
  2nd PCR Forward CM-kneF
gtggatccggtggcggtagcaagaatgaggagggggcgccgcaggggggtggttcgggttcctat

SEQ ID NO: 61 - DNA, 65 bp, artificial sequence
  3rd PCR Forward CM-mdvF
gtggatccggtggcggtagcatggatgtgtttatgaaggggttggggggtggttcgggttcctat

SEQ ID NO: 62 - protein, 218 aa, cucumber mosaic virus
MDKSESTSAGRSRRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 63 - protein, 27 aa, artificial sequence
  aa 2-27 of SEQ ID NO62
DKSESTSAGRSRRRRPRRGSRSAPSSA

SEQ ID NO: 64 - protein, 14 aa, artificial sequence
  tetanus toxoid epitope tt830
QYIKANSKFIGITE

SEQ ID NO: 65 - protein, 13 aa, artificial sequence
  PADRE
AKFVAAWTLKAAA

SEQ ID NO: 66 - protein, 220 aa, artificial sequence
  CMV-Npadr
MAKFVAAWTLKAAARRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 67 - protein, 13 aa, artificial sequence
  HA 307-319
PKYVKQNTLKLAT

SEQ ID NO: 68 - protein, 20 aa, artificial sequence
  HBVnc 50-69
PHHTALRQAILCWGELMTLA

SEQ ID NO: 69 - protein, 21 aa, artificial sequence
  CS 378-398
DIEKKIAKMEKASSVFNVVNS

SEQ ID NO: 70 - protein, 16 aa, artificial sequence
  MT 17-31
YSGPLKAEIAQRLEDV

SEQ ID NO: 71 - protein, 21 aa, artificial sequence
  TT 947-967
FNNFTVSFWLRVPKVSASHLE

SEQ ID NO: 72 - protein, 129 aa, artificial sequence
  Ara h6
AHASAMRRERGRQGDSSSCERQVDGVNLKPCEQHIMQRIMGEQEQYDSYNFGSTRSSDQQQRCCDELNEMENTQRCMCEALQQIMENQCDGLQDRQMVQHFKRELMNLPQQCNFGAPQRCDLDVSGGRC

SEQ ID NO: 73 - protein, 135 aa, artificial sequence
  Ara h201
RQQWELQGDRRCQSQLERANLRPCEQHLMQKIQRDEDSYERDPYSPSQDPYSPSPYDRRGAGSSQHQERCCNELNEFENNQRCMCEALQQIMENQSDRLQGRQQEQQFKRELRNLPQQCGLRAPQRCDLDVESGG

SEQ ID NO: 74 - protein, 514 aa, artificial sequence
  Cry J2
MAMKFIAPMAFVAMQLIIMAAAEDQSAQIMLDSDIEQYLRSNRSLRKVEHSRHDAINIFNVEKYGAVGDGKHDCTEAFSTAWQAACKKPSAMLLVPGNKKFVVNNLFFNGPCQPHFTFKVDGIIAAYQNPASWKNNRIWLQFAKLTGFTLMGKGVIDGQGKQWWAGQCKWVNGREICNDRDRPTAIKFDFSTGLIIQGLKLMNSPEFHLVFGNCEGVKIIGISITAPRDSPNTDGIDIFASKNFHLQKNTIGTGDDCVAIGTGSSNIVIEDLICGPGHGISIGSLGRENSRAEVSYVHVNGAKFIDTQNGLRIKTWQGGSGMASHIIYENVEMINSENPILINQFYCTSASACQNQRSAVQIQDVTYKNIRGTSATAAAIQLKCSDSMPCKNINLSDISLKLTSGKIASCLNDNANGYFSGHVIPACKNLSPSAKRKESKSHKHPKTVMVENMGAYDKGNRTRILLGSRPPNCTNKCHGCSPCKAKLVIVHRIMPQEYYPQRWMCSCHGKIYHP

SEQ ID NO: 75 - protein, 371 aa, artificial sequence
  Amb a1
AEDLQEILPVNETRRLTTSGAYNIIDGCWRGKADWAENRKALADCAQGFGKGTVGGKDGDIYTVTSDLDDDVANPKEGTLRFGAAQNRPLWIIFERDMVIRLDKEMVVNSDKTIDGRGAKVEIINAGFTLNGVKNVIIHNINMHDVKVNPGGLIKSNDGPAAPRAGSDGDAISISGSSQIWIDHCSLSKSVDGLVDAKLGTTRLTVSNSLFTQHQFVLLFGAGDENIEDRGMLATVAFNTFTDNVDQRMPRCRHGFFQVVNNNYDKWGSYAIGGSASPTILSQGNRFCAPDERSKKNVLGRHGEAAAESMKWNWRTNKDVLENGAIFVASGVDPVLTPEQSAGMIPAEPGESALSLTSSAGVLSCQPGAPC

SEQ ID NO: 76 - protein, 70 aa, Felis domesticus
EICPAVKRDVDLFLTGTPDEYVEQVAQYKALPVVLENARILKNCVDAKMTEEDKENALSLLDKIYTSPLC

SEQ ID NO: 77 - protein, 92 aa, Felis domesticus
VKMAETCPIFYDVFFAVANGNELLLDLSLTKVNATEPERTAMKKIQDCYVENGLISRVLDGLVMTTISSSKDCMGEAVQNTVEDLKLNTLGR

SEQ ID NO: 78 - protein, 90 aa, Felis domesticus
VKMAETCPIFYDVFFAVANGNELLLDLSLTKVNATEPERTAMKKIQDCYVENGLISRVLDGLVMIAINEYCMGEAVQNTVEDLKLNTLGR

SEQ ID NO: 79 - protein, 78 aa, Felis domesticus
VKMAETCPIFYDVFFAVANGNELLLDLSLTKVNATEPERTAMKKIQDCYVENGLISRVLDGLVMPSTNIAWVKQFRTP

SEQ ID NO: 80 - protein, 176 aa, artificial sequence
  rFel-2-G3-1
VKMAETCPIFYDVFFAVANGNELLLDLSLTKVNATEPERTAMKKIQDCYVENGLISRVLDGLVMTTISSSKDCMGEAVQNTVEDLKLNTLGRGGGGSGGGGSGGGGSCEICPAVKRDVDLFLTGTPDEYVEQVAQYKALPVVLENARILKNCVDAKMTEEDKENALSVLDKIYTSP

SEQ ID NO: 81 - protein, 176 aa, artificial sequence
  rFel-1-G3-2
CEICPAVKRDVDLFLTGTPDEYVEQVAQYKALPVVLENARILKNCVDAKMTEEDKENALSVLDKIYTSPGGGGSGGGGSGGGGSVKMAETCPIFYDVFFAVANGNELLLDLSLTKVNATEPERTAMKKIQDCYVENGLISRVLDGLVMTTISSSKDCMGEAVQNTVEDLKLNTLGR

SEQ ID NO: 82 - protein, 132 aa, Homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 83 - protein, 115 aa, Homo sapiens
IPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 84 - protein, 114 aa, artificial sequence
  canine IL-5
AVENPMNRLVAETLTLLSTHRTWLIGDGNLMIPTPENKNHQLCIKEVFQGIDTLKNQTAHGEAVDKLFQNLSLIKEHIERQKKRCAGERWRVTKFLDYLQVFLGVINTEWTPES

SEQ ID NO: 85 - protein, 114 aa, artificial sequence
  feline IL-5
AVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTMES

SEQ ID NO: 86 - protein, 129 aa, Homo sapiens [sequence omitted]

SEQ ID NO: 87 - protein, 109 aa, artificial sequence
  canine IL-4
GHNFNITIKEIIKMLNILTARNDSCMELTVKDVFTAPKNTSDKEIFCRAATVLRQIYTHNCSNRYLRGLYRNLSSMANKTCSMNEIKKSTLKDFLERLKVIMQKKYYRH

SEQ ID NO: 88 - protein, 109 aa, artificial sequence
  feline IL-4
QNFNNTLKEIIKTLNILTARNDSCMELTVMDVLAAPKNTSDKEIFCRATTVLRQIYTHHNCSTKFLKGLDRNLSSMANRTCSVNEVKKCTLKDFLERLKAIMQKKYSKH

SEQ ID NO: 89 - protein, 122 aa, Homo sapiens
LTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 90 - protein, 113 aa, artificial sequence
  cIL13 - mature protein
SPSPVTPSPTLKELIEELVNITQNQASLCNGSMVWSVNLTAGMYCAALESLINVSDCSAIQRTQRMLKALCSQKPAAGQISSERSRDTKIEVIQLVKNLLTYVRGVYRHGNFR

SEQ ID NO: 91 - protein, 113 aa, artificial sequence
  feline IL13 - mature protein
SPGPHSRRELKELIEELVNITQNQVSLCNGSMVWSVNLTTGMQYCAALESLINVSDCTAIQRTQRMLKALCTQKPSAGQTASERSRDTKIEVIQLVKNLLNHLRRNFRHGNFK

SEQ ID NO: 92 - protein, 121 aa, artificial sequence
  equine IL13 - mature protein
CLGGLASPAPLPSSMALKELIKELVNITQNQAPLCNGSMVWSVNLTADTYCRALESLSNVSTCSAIQNTRKMLTKLCPHQLSAGQVSSERARDTKIEVIVLVKDLLKNLRKIFHGGKHVDA

SEQ ID NO: 93 - protein, 159 aa, Homo sapiens
SAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 94 - protein, 157 aa, artificial sequence
  canine IL-1alpha
SVAYNFHNNEKYNYIRIIKSQFILNDNLNQSIVRQTGGNYLMTAALQNLDDAVKFDMGAYTSEDSKLPVTLRISKTRLFVSAQNEDEPVLLKEMPETPKTIRDETNLLFFWERHGSKHYFKSVAQPKLFIATQERKLVHMARGQPSITDFRLLETQP

SEQ ID NO: 95 - protein, 158 aa, artificial sequence
  feline IL-1alpha
SVAPNFYSSEKYNYQKIIKSQFILNDNLSQSVIRKAGGKYLAAAALQNLDDAVKFDMGAYTSKEDSKLPVTLRISKTRLFVSAQNEDEPVLLKEMPETPKTIRDETNLLFFWERHGSKNYFKSVAHPKLFIATQEEQLVHMARGLPSVTDFQILETQS

SEQ ID NO: 96 - protein, 158 aa, artificial sequence
  equine IL-1alpha
SVHYNFQSNTKYNFMRIVNHQCTLNDALNQSVIRDTSGQYLATAALNNLDDAVKFDMGAYTSEEDSQLPVTLRISKTRLFVSAQNEDEPVLLKEMPDTPKTIKDETNLLFFWERHGSKNYFKSVAHPKLFIATKQGKLVHMARGQPSITDFQILDNQF

SEQ ID NO: 97 - protein, 175 aa, Homo sapiens
FGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 98 - protein, 169 aa, artificial sequence
  canine IL-33
FMGTSLFEFLMTSFSLVTEYSASLSTYNDQSITFVFEDGSYEIYVEDLRKGQEKDKVLFRYYDSQSPSHETGDDVDGQTLLVNLSPTKDKDFLLHANNEEHSVELQKCENQLPDQAFFLLHRKSSECVSFECKNNPGVFIGVKDNHLALIKVGDQTKDSYIEKTIFKLS

SEQ ID NO: 99 - protein, 170 aa, artificial sequence
  feline IL-33
FGVPMVQKCFGTTDVPSIQEYSASLSTYNDQSITFVFEDGSYEIYVEDLGKDQEKDKVLFRYYDFQSPSRETGDADDGHTLLVNLSPTKDKDFLLHANNKEHSVELQKCKKPLPDQAFFRLHRKSSKCVSFECKNDPGVFIGVKDNHLALIKVEDQTEYFSTENIIFKLS

SEQ ID NO: 100 - protein, 170 aa, artificial sequence
  equine IL-33
FDVPMIQRCARSTAGPIFEEDFASLSTYNDQSISFVIEDGSYEIYIEDLSGQQEKDPVLFRCYPSQCSSSETAGGADGQKLMVNLSPKKYKDFLLHANNKEHSVELQKCENPFPDQAFFLMHKTSSRCVSFECKNNPGVFIGVKDKHLALIKVGEQTEPSSRQNIIFKLS

SEQ ID NO: 101 - protein, 145 aa, Homo sapiens
YSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 102 - protein, 147 aa, artificial sequence
  canine IL-25
KDCTHWPNCCPSKRQDPTHEWLKRDTVLKFPGETTSLTHHPESCKASEDGPLNSRSISPWKYELDRDLNRLPQDLYHARCLCQHCVSLQTGSHMDPLGNSELLYHNQTVFYRRPCPGEQGAPDGYCLEQRLYRVSLACVCVRPRVMA

SEQ ID NO: 103 - protein, 147 aa, artificial sequence
  feline IL-25
KDCIHWSSCCPSKGQNPTHEWLTENTVLMTPPESASLIHSLESCRASEDGPLNSRSISPWRYELDRDLNRLPQDLYHARCLCPHCVSLQTGSHMDPLGNSELLYHNQTVFYRRPCPGEQGTRDGYCLEQRLYRVSLACVCVRPRVMA

SEQ ID NO: 104 - protein, 147 aa, artificial sequence
  equine IL-25
KECTHWPSCCPKKGQDPIEEWLKWSTAHVPPPETANLAHHPESCRASEDGPLNSRSISPWRYELDRDLNRLPQDLYHARCLCPHCVSLQTGSHMDPLGNSELLYHNQTVFYRRPCPGKRGAHDGYCLERRLYRVSLACVCVRPRVMA

SEQ ID NO: 105 - protein, 153 aa, Homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 106 - protein, 141 aa, Homo sapiens [sequence omitted]

SEQ ID NO: 107 - protein, 136 aa, artificial sequence
  canine IL-31
SHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQ

SEQ ID NO: 108 - protein, 136 aa, artificial sequence
  feline IL-31
SHMAPAHRLQPSDVRKIILELRPMSKGLLQDYLKKEIGLPESNHSSLPCLSSDSQLPHINGSAILPYFRAIRPLSDKNTIDKIIEQLDKLKFQREPEAKVSMPADNFERKNFILAVLQQFSACLEHVLQSLNSGPQ

SEQ ID NO: 109 - protein, 130 aa, artificial sequence
  equine IL-31
SHTGPIYQLQPKEIQAIIVELQNLSKKLLDDYLNKEKGVQKFDSDLPSCFTSDSQAPGNINSSAILPYFKAISPSLNNDKSLYIIEQLDKLNFQNAPETEVSMPTDNFERKRFILTILRWFSNCLEHRAQ

SEQ ID NO: 110 - protein, 131 aa, Homo sapiens
YDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 111 - protein, 127 aa, artificial sequence
  canine TLSP
YNFIDCDFEKIRWKYQEVIYQALEKYMDGTRSTEFSHPVYCADPPDCLARIERLTLHRIRGCASGAREAFAEGTVAALAAECPGYAAAPINNTQAKKKRKKRGVTTNKCREQVAHLIGLWRRFSRIS

SEQ ID NO: 112 - protein, 127 aa, artificial sequence
  feline TLSP
YNFTDCDFGKIRKKYQEVIYQALNKYMNGVKSTEFNNYIYCEDPPDCLAKIDLITFYPTHGCTTLAKEIFAIRTNATLTLQCPGYSGTQINNTQAKKKRKKRQVTTNKCREQVSHLMELWRRFSRIS

SEQ ID NO: 113 - protein, 125 aa, artificial sequence
  equine TLSP
YDFTDCNFAKIEEEYQNVIFLALKEYMNGIKSTQFNHTVYCGDRPSCLTEIERLTFKSSQCLSLAKKIVTATNATLNSHCPGHSGIQINNTQAMKKRKKREVTTNKCLKQVSNLIELWRYFSRSQ

SEQ ID NO: 114 - protein, 42 aa, Homo sapiens [sequence omitted]

SEQ ID NO: 115 - protein, 5 aa, artificial sequence
  Abeta 2-6
AEFRH

SEQ ID NO: 116 - protein, 5 aa, artificial sequence
  Abeta 3-7
EFRHD

SEQ ID NO: 117 - protein, 7 aa, artificial sequence
  alpha-syn kne*
KNEEGAP

SEQ ID NO: 118 - protein, 95 aa, artificial sequence
  cow myostatin
CCRYPLTVDFEAFGWDWIIAPKRYKANYCSGECEFVFLQKYPHTHLVHQANPRGSAGPCCTPTKMSPINMLYFNGEGQIIYGKIPAMVVDRCGCS

SEQ ID NO: 119 - protein, 165 aa, artificial sequence
  plasmodium vivax
DRADGQPAGDRADGQPAGDRAAGQPAGDRAAGQPAGDRADGQPAGDRADGQPAGAPGANQEGGAAAPGANQEGGAAAPGANQEGGAAAPGANQEGGAAAPGANQEGGAAAPGANQEGGAAANGAGNQPGANGAGNQPGANGAGNQPGANGAGNQPGANGAGNQPG

SEQ ID NO: 120 - protein, 24 aa, artificial sequence
  Influenza M2 peptide
SLLTEVETPIRNEWGCRCNDSSDG

SEQ ID NO: 121 - DNA, 42 bp, artificial sequence
  Reverse Cmded-BamR
ggatccatcctcgtcacctcctccagatccacctccaccact

SEQ ID NO: 122 - DNA, 42 bp, artificial sequence
  Forward CMded-BamF
ggatcccagactagtgacgaggatggaggaggtggaagcgga

SEQ ID NO: 123 - DNA, 31 bp, artificial sequence
  Forward I5-BamF
ggatccattgcagttcagagcccgatgaatc

SEQ ID NO: 124 - DNA, 27 bp, artificial sequence
  Reverse I5-SpeR
actagtgctttccatggtccattcggt

SEQ ID NO: 125 - protein, 115 aa, Felis catus
IAVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTMES

SEQ ID NO: 126 - protein, 20 aa, artificial sequence
  18 aa linker GSED
GSGGGGSGGGGSGGGDEDGS

SEQ ID NO: 127 - protein, 15 aa, artificial sequence
  15 aa linker GSED
TSDEDGGGGSGGGGS

SEQ ID NO: 128 - protein, 370 aa, artificial sequence
  CMV-Ntt830-fel-IL-5
MGQYIKANSKFIGITERRRRPRRGSRSAPSSADANFRVLSQQLSRLNKTLAAGRPTINHPTFVGSERCKPGYTFTSITLKPPKIDRGSGGGGSGGGGSGGGDEDGSIAVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTMESTSDEDGGGGSGGGGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 129 - DNA, 1122 bp, artificial sequence
  CMV-Ntt830-fel-IL-5
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatctggaggaggtggtagtggtggaggtggatctggaggaggtgacgaggatggatccattgcagttcagagcccgatgaatcgtctggttgcagaaaccctggcactgctgagcacccatcgtaccctgctgattggtgatggtaatctgatgattccgacaccggaacataataatcatcagctgtgtatcgaagaagtgtttcagggcattgataccctgaaaaatcgtaccgttccgggtgatgcagttgaaaaactgtttcgtaatctgagcctgatcaaagaacatatcgatcgccagaaaaaaaagtgtggtggtgaacgttggcgtgtgaaaaaattcctggattatctgcaggtttttctgggcgtgattaataccgaatggaccatggaaagcactagtgacgaggatggaggaggtggaagcggaggaggtggatcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgttctcgtgtattcaaaagacgatgcactcgagacagacgagttagtacttcatgttgacgtcgagcaccaacgtattcccacatctggggtgctcccagtttgataagctt

SEQ ID NO: 130 - DNA, 62 bp, artificial sequence
  Reverse 2xIL5-gsKpnR
ctccggaaccaccgccaccgctaccaccaccaccgctttccatggtccattcggtattaatc

SEQ ID NO: 131 - DNA, 44 bp, artificial sequence
  Forward 2xIL5-gsKpnF
ttccggaggtggcggtagcattgcagttcagagcccgatgaatc

SEQ ID NO: 132 - protein, 500 aa, artificial sequence
  CMV-Ntt830-2xfel-IL-5
MGQYIKANSKFIGITERRRRPRRGSRSAPSSADANFRVLSQQLSRLNKTLAAGRPTINHPTFVGSERCKPGYTFTSITLKPPKIDRGSGGGGSGGGGSGGGDEDGSIAVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTMESGGGGSGGGGSGGGGSIAVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTMESTSDEDGGGGSGGGGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 133 - DNA, 1512 bp, artificial sequence
  CMV-Ntt830-2xfel-IL-5
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatctggaggaggtggtagtggtggaggtggatctggaggaggtgacgaggatggatccattgcagttcagagcccgatgaatcgtctggttgcagaaaccctggcactgctgagcacccatcgtaccctgctgattggtgatggtaatctgatgattccgacaccggaacataataatcatcagctgtgtatcgaagaagtgtttcagggcattgataccctgaaaaatcgtaccgttccgggtgatgcagttgaaaaactgtttcgtaatctgagcctgatcaaagaacatatcgatcgccagaaaaaaaagtgtggtggtgaacgttggcgtgtgaaaaaattcctggattatctgcaggtttttctgggcgtgattaataccgaatggaccatggaaagcggtggtggtggtagcggtggcggtggttccggaggtggcggtagcattgcagttcagagcccgatgaatcgtctggttgcagaaaccctggcactgctgagcacccatcgtaccctgctgattggtgatggtaatctgatgattccgacaccggaacataataatcatcagctgtgtatcgaagaagtgtttcagggcattgataccctgaaaaatcgtaccgttccgggtgatgcagttgaaaaactgtttcgtaatctgagcctgatcaaagaacatatcgatcgccagaaaaaaaagtgtggtggtgaacgttggcgtgtgaaaaaattcctggattatctgcaggtttttctgggcgtgattaataccgaatggaccatggaaagcactagtgacgaggatggaggaggtggaagcggaggaggtggatcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgttctcgtgtattcaaaagacgatgcactcgagacagacgagttagtacttcatgttgacgtcgagcaccaacgtattcccacatctggggtgctcccagtttgataagctt

SEQ ID NO: 134 - protein, 152 aa, artificial sequence
  canine IL-1b
AAMQSVDCKLQDISHKYLVLSNSYELRALHLNGENVNKQVVFHMSFVHGDESNNKIPVVLGIKQKNLYLSCVMKDGKPTLQLEKVDPKVYPKRKMEKRFVFNKIEIKNTVEFESSQYPNWYISTSQVEGMPVFLGNTRGGQDITDFTMEFSS

SEQ ID NO: 135 - protein, 407 aa, artificial sequence
  CMV-Ntt830-cIL-1b
MGQYIKANSKFIGITERRRRPRRGSRSAPSSADANFRVLSQQLSRLNKTLAAGRPTINHPTFVGSERCKPGYTFTSITLKPPKIDRGSGGGGSGGGGSGGGDEDGSAAMQSVDCKLQDISHKYLVLSNSYELRALHLNGENVNKQVVFHMSFVHGDESNNKIPVVLGIKQKNLYLSCVMKDGKPTLQLEKVDPKVYPKRKMEKRFVFNKIEIKNTVEFESSQYPNWYISTSQVEGMPVFLGNTRGGQDITDFTMEFSSTSDEDGGGGSGGGGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 136 - DNA, 1233 bp, artificial sequence
  CMV-Ntt830-cIL-1b
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatctggaggaggtggtagtggtggaggtggatctggaggaggtgacgaggatggatccgcagccatgcaatcggtggactgcaagttacaggacataagccacaaatacctggtgctgtctaactcttatgagcttcgggctctccacctcaatggggaaaatgtgaacaaacaagtggtgttccacatgagctttgtgcacggggatgaaagtaataacaagatacctgtggtcttgggcatcaaacaaaagaatctgtacctgtcctgtgtgatgaaggatggaaagcccaccctacagctagagaaggtagaccccaaagtctacccaaagaggaagatggaaaagcgatttgtcttcaacaagatagaaatcaagaacacagtggaatttgagtcttctcagtaccctaactggtacatcagcacctctcaagtcgaaggaatgcctgtcttcctaggaaataccagaggtggccaggatataactgacttcacgatggaattctcttccactagtgacgaggatggaggaggtggaagcggaggaggtggatcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgttctcgtgtattcaaaagacgatgcactcgagacagacgagttagtacttcatgttgacgtcgagcaccaacgtattcccacatctggggtgctcccagtttgataagctt

SEQ ID NO: 137 - DNA, 48 bp, artificial sequence
  Forward CM-BamSpeF
tggatcctctactagtggaggaggtggaagcggaggaggtggatctta

SEQ ID NO: 138 - protein, 11 aa, artificial sequence
  11 amino acid GST linker
TSGGGGSGGGG

SEQ ID NO: 139 - protein, 363 aa, artificial sequence
  CMV-Ntt830-fel-IL-5*
MGQYIKANSKFIGITERRRRPRRGSRSAPSSADANFRVLSQQLSRLNKTLAAGRPTINHPTFVGSERCKPGYTFTSITLKPPKIDRGSGGGGSGGGGSGGGGSIAVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTMESTSGGGGSGGGGYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 140 - DNA, 1101 bp, artificial sequence
  CMV-Ntt830-fel-IL-5*
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatctggaggaggtggtagtggtggaggtggatctggaggaggtggatccattgcagttcagagcccgatgaatcgtctggttgcagaaaccctggcactgctgagcacccatcgtaccctgctgattggtgatggtaatctgatgattccgacaccggaacataataatcatcagctgtgtatcgaagaagtgtttcagggcattgataccctgaaaaatcgtaccgttccgggtgatgcagttgaaaaactgtttcgtaatctgagcctgatcaaagaacatatcgatcgccagaaaaaaaagtgtggtggtgaacgttggcgtgtgaaaaaattcctggattatctgcaggtttttctgggcgtgattaataccgaatggaccatggaaagcactagtggaggaggtggaagcggaggaggtggatattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 141 - protein, 115 aa, artificial sequence
  fel IL-5
IAVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTIEG

SEQ ID NO: 142 - protein, 109 aa, artificial sequence
  fel IL-5
IAVQSPMNRLVAETLALLSTHRTLLIGDGNLMIPTPEHNNHQLCIEEVFQGIDTLKNRTVPGDAVEKLFRNLSLIKEHIDRQKKNFGGKKWKVKNFLNYLQFFLGLLNT

SEQ ID NO: 143 - protein, 152 aa, artificial sequence
  canine IL-1b
AAMQSVDCKLQGISHKYLVLSNSYELRALHLNGENVNKQVVFHMSFVHGDESNNKIPVVLGIKQKNLYLSCVMKDGKPTLQLEKVDPKVYPKRKMEKRFVFNKIEIKNTVEFESSQYPNWYISTSQVEGMPVFLGNTRGGQDITDFTMEFSS

SEQ ID NO: 144 - protein, 152 aa, artificial sequence
  canine IL-1b
AALPSVDCTLQDINHKYLVLSNSYELRALHLNGENVNKQVVFHMSFVHGDESNYKIPVVLGIKQKNLYLSCVMKDGKPTLQLEKVDPKVYPKRKMEKRFVFNKIEIKNTVEFESSQYPNWYISTSQVEGMPVFLGNTKGGQDITDFTMEFSS

SEQ ID NO: 145 - protein, 152 aa, artificial sequence
  fel IlL-1b
AAIQSQDYTFRDISQKSLVLSGSYELRALHLNGQNMNQQVVFRMSFVHGEENSKKIPVVLCIKKNNLYLSCVMKDGKPTLQLEMLDPKVYPKKKMEKRFVFNKTEIKGNVEFESSQFPNWYISTSQAEEMPVFLGNTKGGQDITDFIMESAS

(19) United States
(12) Patent Application Publication
(10) Pub. No.: US 2022/0073946 A1
(43) Pub. Date: Mar. 10, 2022

(54) VIRUS-LIKE PARTICLES OF CMV MODIFIED BY FUSION

(71) Applicant: SAIBA AG, Pfäffikon (CH)

(72) Inventor: Andris ZELTINS, Riga (LV)

(21) Appl. No.: 17/414,730

(22) PCT Filed: Dec. 20, 2019
(86) PCT No.: PCT/EP2019/086788

§ 371 Date: Jun. 16, 2021

(30) Foreign Application Priority Data
Dec. 20, 2018 (EP) 18214639.9

(51) Int. Cl. C12N 15/86; C07K 14/005

(52) U.S. Cl. CPC C12N 15/86; C07K 14/005; C07K 2319/00; C12N 2770/14043; C12N 2770/14023

(57) ABSTRACT

The present invention relates to a modified virus-like particle (VLP) of cucumber mosaic virus (CMV) comprising at least one fusion protein, wherein said at least one fusion protein comprises, or preferably consists of b) a chimeric CMV polypeptide, wherein said chimeric CMV polypeptide comprises, or preferably consists of (iii) a CMV polypeptide, wherein said CMV polypeptide comprises, or preferably consists of, a coat protein of CMV, wherein preferably said coat protein of CMV comprises, or preferably consists of, SEQ ID NO:62; or an amino acid sequence having a sequence identity of at least 75%, preferably of at least 80%, more preferably of at least 85%, again further preferably of at least 90%, again more preferably of at least 95%, still further preferably of at least 98% and still again further more preferably of at least 99% with SEQ ID NO:62; and (iv) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and (iii) a T helper cell epitope, wherein said T helper cell epitope replaces a N-terminal region of said CMV polypeptide, and wherein preferably said N-terminal region of said CMV polypeptide corresponds to amino acids 2-12 of SEQ ID NO:62.

The present invention relates to modified virus-like particles of plant virus Cucumber Mosaic Virus (CMV), and in particular to modified VLPs of CMV comprising chimeric CMV polypeptides which comprises antigenic polypeptides inserted into CMV polypeptides at a specific position and further comprises Th cell epitopes replacing a N-terminal region of said CMV polypeptides. Furthermore, these modified VLPs serve as, preferably, vaccine platform, for generating immune responses, in particular antibody responses, against said antigenic polypeptides fused into said CMV polypeptides. Moreover, the present invention relates to modified virus-like particles being mosaic virus-like particle comprising said chimeric CMV polypeptides comprising the in-fused antigenic polypeptides and further CMV proteins not comprising said antigenic polypeptides.

### RELATED ART

Plant viruses and virus-like particles (VLPs) derived therefrom have recently attracted attention, mainly due to the role of plants as an economical and speedy alternative platform for producing VLP vaccines in the light of their ability to provide distinctive posttranslational modifications, cost-effectiveness, beneficial safety profiles, production speed and scalability (Chen Q and Lai H, Human Vaccines & Immunotherapeutics (2013) 9:26-49; Zeltins A, Mol Biotechnol (2013) 53:92-107). Furthermore, in particular VLPs of plant viruses have been considered as carrier structure for presentation of different antigens on their surfaces aiming at eliciting strong immune responses similar to those observed with infectious mammalian viruses (Balke I, et al., Adv. Drug Deliv. Rev. (2018) https://doi.org/10.1016/j.addr.2018.08.007).

Cucumber Mosaic Virus (CMV, family Bromoviridae, genus Cucumovirus) is a linear positive-sense isodiametric plant virus with an extremely wide host range. The virus genome consists of three single-stranded RNAs (RNA1, RNA2 and RNA3), the coat protein (CP) gene being present both in the genomic RNA3 (about 2200 nt) and in the subgenomic RNA4 (about 1000 nt). The capsid comprises 180 copies of a single protein species of about 25 kDa. There are a plurality of different strains known from CMV associated with variable symptoms related to the host plant such as CMV-B strain, CMV-C strains CMV-D strain, CMV-L strains, CMV-S strain, CMV-T strain, CMV-WL-strain, CMV-V strains, CMV-Fny strain, CMV-Ix strain, CMV-Q strain, CMV-R strain or the like (Carrére I, et al., Arch Virol (1999) 144:1846-1857; Edwards M C, et al., Phytopathology 81983) 73:1117-1120; www.dpvweb.net).

Recently, a vaccine platform based on CMV VLPs has been described using chemical linker coupling technology to present different antigens on their surface. The described CMV VLPs are derived from modified CPs of CMV with inserted T-cell stimulating epitopes (A. Zeltins, et al. Vaccines 2 (2017) 30; WO2016/062720).

Chimeric forms of CMV have also been engineered to function as a presentation system and to express on their outer surface epitopes derived from the hepatitis C virus (HCV). In detail, a CMV pseudo-recombinant form CMV-D/S has been engineered to carry genomic RNA3 from the CMV-S strain and RNA1 and RNA2 from the CMV-D strain. This system developed virus symptoms such as mild mosaic and vein clearing in Xanthi tobacco plants after inoculation. The CP gene was then engineered in different positions, to encode a Hepatitis C virus (HCV) epitope. The selected peptide was the so-called R9 mimotope, a synthetic peptide of 27 amino acids derived from many hypervariable region 1 (HVR1) sequences of the HCV envelope protein E2. The selection of the insertion points of the R9 mimotope into the CMV gene was made taking essential factors into account: i) the need to protect the N-terminal region of the CMV coat protein (containing a high concentration of basic amino acids, known as an internal R-domain involved in protein-RNA interactions stabilizing CMV (Wikoff W R, et al., Virology (1997) 232: 91-97) characterized by an unusual N-terminal helix with an additional stabilizing role in the capsid (Smith T J, et al., J Virol (2000) 74: 7578-7686); ii) the surface location of the foreign epitope to increase the chance of its putative immunogenic capability; iii) the availability of mutagenesis routes able to produce the modified clones. On the basis of these considerations, the insertion of the R9 mimotopes has been effected at different locations within the CP gene of CMV-S RNA3 (AF063610, www.dpvweb.net). For the insertion of one single R9 mimotope within said CP gene, the R9 mimotope nucleotide sequence was inserted in positions 253, 475, 529 of said CP gene, whereas for the insertion of two R9 mimotopes, the R9 mimotope nucleotide sequence was inserted in position 392 and 529. Even though the so prepared chimeric CMVs retained their ability to spread systemically in the host plant, a lower virus extraction yield was obtained in case of the first two insertion sites. Thus, to guarantee higher concentrations of virus particles in infected tissues, the mosaic CMV containing the R9 mimotope inserted at position 529 of the CP gene was selected and tested for HCV patient serum reactivity. Serum samples from 60 patients with chronic hepatitis C displayed a significant immunoreactivity to crude plant extracts infected with said chimeric CMV (Natilla A, et al., Arch Virol (2004) 149:137-154; Piazzolla G, et al., J Clin Immunol (2005) 25:142-152; Nuzzaci M, et al., Arch Virol (2007) 152:915-928; Nuzzaci M, et al., Journal of Virological Methods (2009) 155:118-121; Nuzzaci M, et al., Journal of Virological Methods (2010) 165:211-215; Piazzolla G, et al., J Clin Immunol (2012) 32:866-876).

Furthermore, cucumber mosaic virus based expression systems have been described either as potential vaccine against Alzheimer's disease as well as for the production of porcine circovirus specific vaccines (Vitti A, et al., J Virol Methods (2010) 169:332-340; Gellert A, et al., PloS ONE (2012) 7(12): e52688). In detail, chimeric constructs bearing different 11-15 amino acids long Aβ-derived fragments in positions 248, 392 or 529 of the CMV coat protein (CP) gene were created and the viral products proved to be able to replicate in their natural host. On the other hand, porcine circovirus type 2 (PCV2) capsid protein epitopes of up to a length of 20 amino acids were integrated into the plant virus coat protein of cucumber mosaic virus (CMV)-R strain after amino acid position 131. This insertion point 131-132 is located in the middle of the βE-αEF loop of the CMV CP and it has been concluded that this position has the advantage that the inserted epitopes form a tripartite group in the middle of the CMV CP trimers allowing the production of antibodies more efficiently.

Furthermore, the generation of chimeric virus-like particles (VLPs) of CMV has also been described, even though the expression of viral capsid proteins (CPs) of CMV by the widely used traditional E. coli expression system led only to insoluble inclusion bodies or a very low quantity of soluble proteins (Xu Y, et al., Chem Commun (2008) 49-51). On the other hand, chimeric CMV coat proteins expressed from a potato virus X (PVX)-based vector were capable of assembling into VLPs (Natilla, A, et al., Arch Virol (2006) 151:1373-1386; Natilla, A, et al., Protein Expression and Purification (2008) 59:117-121; Chen Q and Lai H, Human Vaccines & Immunotherapeutics (2013) 9: 26-49). The chimeric CMV coat proteins comprise 17-25 amino acids long epitopes of Newcastle disease virus (NDV) genetically fused into the internal βH-βI (motif 5) loop of the CMV CP which corresponds to amino acids 194-199 thereof (He X, et al (1998) J Gen Virol 79: 3145-3153).

Even though progress has been made in the course of the development of VLP based vaccines, there is still a need for further distinct VLP systems. In particular, vaccines induce variable antibody responses in immunized subjects and individuals often spanning a range of more than 100-fold variation. In addition, some vaccines, such as the vaccine against Hepatitis B, suffer from a certain number of non-responders. Non-responsiveness is associated with certain MHC class II molecules and failure to induce good T helper (Th) cell responses is believed to be responsible for poor antibody responses seen in these individuals (Goncalves L, et al., Virology (2004) 326:20-28). Furthermore, elderly people mount poor antibody responses in general and poor Th cell responses are again thought to be the cause of the inefficient antibody responses. Therefore, vaccines inducing good Th cell responses in essentially all subjects and individuals are an important goal in the field of vaccine development. Moreover, one further associated very important problem yet to be solved during the vaccine construction process is antigen spatial conformation. For vaccine construction, it is important to achieve optimal peptide presentation on the particle surface without affecting the overall viral structure. This becomes evident since only in exceptional cases, VLPs are able to accommodate more than 50 or 70 amino acids-long protein domains or even still longer and retain the typical VLP morphology (I. Balke et al., Adv. Drug Deliv. Rev. (2018), https://doi.org/10.1016/j.addr.2018.08.007; I. Kalnciema, et al. Mol. Biotechnol. 52 (2012) 129-139).

### SUMMARY OF THE INVENTION

We have now surprisingly found that antigenic polypeptides of various and very different length and nature can be inserted at a specific position of CMV polypeptides already modified by the incorporation of a T helper cell epitope, and that said resulting fusion proteins are still capable of forming and assembling to modified virus-like particles (VLPs) which are, in addition, highly immunogenic. Preferably and further surprisingly, mosaic modified virus-like particles have been generated comprising said described fusion proteins with the in-fused antigenic polypeptides and further comprising CMV proteins with no such in-fused antigenic polypeptides. These mosaic modified CMV VLPs were found to be highly beneficial for and allow even the incorporation of antigenic polypeptides of very high length such as the incorporation of antigenic polypeptides of up to more than 200 amino acids in length.

In a first aspect, the present invention provides a modified virus-like particle (VLP) of cucumber mosaic virus (CMV) comprising at least one fusion protein, wherein said at least one fusion protein comprises, or preferably consists of,
a) a chimeric CMV polypeptide, wherein said chimeric CMV polypeptide comprises, or preferably consists of
   (i) a CMV polypeptide, wherein said CMV polypeptide comprises, or preferably consists of, a coat protein of CMV, wherein preferably said coat protein of CMV comprises, or preferably consists of, SEQ ID NO:62; or an amino acid sequence having a sequence identity of at least 75%, preferably of at least 80%, more preferably of at least 85%, again further preferably of at least 90%, again more preferably of at least 95%, still further preferably of at least 98% and still again further more preferably of at least 99% with SEQ ID NO:62; and
   (ii) an antigenic polypeptide, wherein said antigenic polypeptide is inserted into said CMV polypeptide,
   wherein said insertion of said antigenic polypeptide is between amino acid residues of said CMV polypeptide corresponding to amino acid residues of position 84 and position 85 of SEQ ID NO:62; and
   (iii) a T helper cell epitope, wherein said T helper cell epitope replaces a N-terminal region of said CMV polypeptide, and wherein preferably said N-terminal region of said CMV polypeptide corresponds to amino acids 2-12 of SEQ ID NO:62.

In a further aspect, the present invention provides a modified VLP of CMV comprising at least one fusion protein, wherein said at least one fusion protein comprises, or preferably consists of,
a) a chimeric CMV polypeptide, wherein said chimeric CMV polypeptide comprises, or preferably consists of
   (i) a CMV polypeptide, wherein said CMV polypeptide comprises, or preferably consists of, a coat protein of CMV, wherein preferably said coat protein of CMV comprises, or preferably consists of, SEQ ID NO:62; or an amino acid sequence having a sequence identity of at least 75%, preferably of at least 80%, more preferably of at least 85%, again further preferably of at least 90%, again more preferably of at least 95%, still further preferably of at least 98% and still again further more preferably of at least 99% with said coat protein, preferably with said SEQ ID NO:62; and
   (ii) an antigenic polypeptide, wherein said antigenic polypeptide is inserted into said CMV polypeptide,
   wherein said insertion of said antigenic polypeptide is between amino acid residues of said CMV polypeptide corresponding to amino acid residues of position 84 and position 85 of SEQ ID NO:62; and
   (iii) a first amino acid linker, preferably a first amino acid linker and a second amino acid linker, wherein said first amino acid linker is positioned at the N- or at the C-terminus of said antigenic polypeptide, and wherein preferably said first amino acid linker is selected from the group consisting of:
      (a.) a polyglycine linker (Gly)ₙ of a length of n=2-10;
      (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and
      (c.) an amino acid linker comprising at least one Gly, at least one Ser, and at least one amino acid selected from Thr, Ala, Lys, Asp and Glu.

In another aspect, the present invention provides a modified virus-like particle (VLP) of cucumber mosaic virus (CMV) comprising
(a) at least one fusion protein, wherein said at least one fusion protein comprises, or preferably consists of,
   a) a chimeric CMV polypeptide, wherein said chimeric CMV polypeptide comprises, or preferably consists of
      (i) a CMV polypeptide, wherein said CMV polypeptide comprises, or preferably consists of, a coat protein of CMV, wherein preferably said coat protein of CMV comprises, or preferably consists of, SEQ ID NO:62; or an amino acid sequence having a sequence identity of at least 75%, preferably of at least 80%, more preferably of at least 85%, again further preferably of at least 90%, again more preferably of at least 95%, still further preferably of at least 98% and still again further more preferably of at least 99% with SEQ ID NO:62; and
      (ii) an antigenic polypeptide, wherein said antigenic polypeptide is inserted into said CMV polypeptide,
      wherein said insertion of said antigenic polypeptide is between amino acid residues of said CMV polypeptide corresponding to amino acid residues of position 84 and position 85 of SEQ ID NO:62; and
(b) at least one CMV protein, wherein said CMV protein comprises, or preferably consists of, a coat protein of CMV, wherein preferably said coat protein of CMV comprises, or preferably consists of, SEQ ID NO:62; or an amino acid sequence having a sequence identity of at least 75%, preferably of at least 80%, more preferably of at least 85%, again further preferably of at least 90%, again more preferably of at least 95%, still further preferably of at least 98% and still again further more preferably of at least 99% with SEQ ID NO:62, and wherein said CMV protein is optionally modified by a T helper cell epitope, wherein said T helper cell epitope replaces a N-terminal region of said CMV protein, and wherein preferably said N-terminal region of said CMV protein corresponds to amino acids 2-12 of SEQ ID NO:62.

Further aspects and embodiments of the present invention will be become apparent as this description continues.

### BRIEF DESCRIPTION OF FIGURES

FIG. 1: Description of pET-CMV-Ntt830-Ab36 plasmid map with single-cut restriction enzyme sites. All other maps (pET-CMV-Ntt830-Ab15; pET-CMV-Ntt830-Ab16; pET-CMV-Ntt830-Ab17) have essentially the same sequences and gene organisations; they differ only in nucleotide sequences of coding for amyloid (beta) peptides.

FIG. 2A: SDS-PAGE gel analysis of the purification of the VLP derived from the expression of CMV-Ntt830-Ab36. M—protein size marker PageRuler (Thermo Fisher Scientific, #26620); S—soluble proteins in E. coli C2566 cells after 18 h cultivation at 20° C. in cell extract before sucrose gradient (20-60%); P—insoluble proteins; 1-6—sucrose gradient fractions (from 60% at the bottom of tube to 0% at the top. The asterisc (*) denotes the relative position of the corresponding CMV-Ntt830-Ab36 chimeric CMV polypeptide in SDS/PAGE gel.

FIG. 2B: SDS-PAGE gel analysis of the purification of the VLP derived from the expression of CMV-Ntt830-Ab15. M—protein size marker PageRuler (Thermo Fisher Scientific, #26620); S—soluble proteins in E. coli C2566 cells after 18 h cultivation at 20° C. in cell extract before sucrose gradient (20-60%); P—insoluble proteins; 1-6—sucrose gradient fractions (from 60% at the bottom of tube to 0% at the top. The asterisc (*) denotes the relative position of the corresponding CMV-Ntt830-Ab15 chimeric CMV polypeptide in SDS/PAGE gel.

FIG. 2C: SDS-PAGE gel analysis of the purification of the VLP derived from the expression of CMV-Ntt830-Ab16. M—protein size marker PageRuler (Thermo Fisher Scientific, #26620); S—soluble proteins in E. coli C2566 cells after 18 h cultivation at 20° C. in cell extract before sucrose gradient (20-60%); P—insoluble proteins; 1-6—sucrose gradient fractions (from 60% at the bottom of tube to 0% at the top. The asterisc (*) denotes the relative position of the corresponding CMV-Ntt830-Ab16 chimeric CMV polypeptide in SDS/PAGE gel.

FIG. 2D: SDS-PAGE gel analysis of the purification of the VLP derived from the expression of CMV-Ntt830-Ab17. M—protein size marker PageRuler (Thermo Fisher Scientific, #26620); S—soluble proteins in E. coli C2566 cells after 18 h cultivation at 20° C. in cell extract before sucrose gradient (20-60%); P—insoluble proteins; 1-6—sucrose gradient fractions (from 60% at the bottom of tube to 0% at the top. The asterisc (*) denotes the relative position of the corresponding CMV-Ntt830-Ab17 chimeric CMV polypeptide in SDS/PAGE gel.

FIG. 3A: Electron microscopy analysis of purified VLP derived from the expression of CMV-Ntt830-Ab36. White horizontal bar corresponds to 100 nm.

FIG. 3B: Electron microscopy analysis of purified VLP derived from the expression of CMV-Ntt830-Ab15. White horizontal bar corresponds to 100 nm.

FIG. 3C: Electron microscopy analysis of purified VLP derived from the expression of CMV-Ntt830-Ab16. White horizontal bar corresponds to 100 nm.

FIG. 3D: Electron microscopy analysis of purified VLP derived from the expression of CMV-Ntt830-Ab17. White horizontal bar corresponds to 100 nm.

FIG. 4: Binding of monoclonal antibodies with the variable region sequence of Aducanumab to CMV-Ntt830-Ab36 VLP, Abeta1-42 peptide and to CMV-Ntt830 VLP.

FIG. 5A: CMV-Ntt830-Ab16 VLP, CMV-Ntt830-Ab17 VLP and CMV-Ntt830-Ab36 VLP induce antibodies that recognize full length Abeta1-42 peptide.

FIG. 5B: CMV-Ntt830-Ab36 VLP induces antibodies recognizing plaques in brains of Alzheimer's patients FIG. 6: Description of pETDu-CMVB2xArah202-CMV-tt plasmid map with single-cut restriction enzyme sites. The expression vector ensures simultaneous synthesis of CMV-Ntt830-Arah202 and unmodified CMV-Ntt830.

FIG. 7A: SDS-PAGE gel of purification of mosaic VLP comprising CMV-Ntt830-Arah202 and unmodified CMV-Ntt830. M—protein size marker PageRuler (Thermo Fisher Scientific, #26620); T—total proteins in E. coli C2566/pETDu-CMVxArah202-CMVtt cells after 18 h cultivation at 20° C.; S—soluble proteins in cell extract before sucrose gradient (20-60%); P—insoluble proteins; 1-6—sucrose gradient fractions (from 60% at the bottom of tube to 0% at the top). The asterisc (*) denotes the relative position of CMV-Ntt830-Arah202 chimeric CMV polypeptide in gel.

FIG. 7B: Western blot analysis of purification of mosaic VLPs comprising CMV-Ntt830-Arah202 and unmodified CMV-Ntt830. M—protein size marker PageRuler (Thermo Fisher Scientific, #26620); T—total proteins in E. coli C2566/pETDu-CMVxArah202-CMVtt cells after 18 h cultivation at 20° C.; S—soluble proteins in cell extract before sucrose gradient (20-60%); P—insoluble proteins; 1-6—sucrose gradient fractions (from 60% at the bottom of tube to 0% at the top). For Western blot, rabbit pAbs against Arah2 (1:1000; Indoor Biotechologies, #PA-AH2) were used. Western blot confirms the presence of Ara-h202 in CMV VLP fractions. The asterisc (*) denotes the relative position of CMV-Ntt830-Arah202 fusion protein in blot.

FIG. 8A: SDS-PAGE gel analysis of purification of mosaic VLPs comprising CMV-Ntt830-Arah202 and unmodified CMV-Ntt830. M—protein size marker PageRuler (Thermo Fisher Scientific, #26620), 1—purified CMV-Ntt830 VLPs (control sample) 2—purified mosaic VLPs comprising CMV-Ntt830-Arah202 and unmodified CMV-Ntt830.

FIG. 8B: Electron microscopy analysis of purification of mosaic VLPs comprising CMV-Ntt830-Arah202 and unmodified CMV-Ntt830. Electron microscopy image of purified mosaic VLPs comprising CMV-Ntt830-Arah202 and unmodified CMV-Ntt830. White horizontal bar corresponds to 100 nm.

FIG. 9: ELISA for IgG to recombinant Ara-h202 14 days after immunization with Ara-h202 or mosaic VLPs comprising CMV-Ntt830-Arah202 and unmodified CMV-Ntt830 (CMV-M-Arah202).

FIG. 10A: Experimental design to investigate the protective effect of vaccination with CMV-M-Arah202 against allergic systemic and local reaction

FIG. 10B: Protection from systemic and local challenge. Systemic challenge with peanut extract.

FIG. 10C: Protection from systemic and local challenge. Skin prick test with peanut extract.

FIG. 11: Description of pET28-CMVBxFeld1-CMV-tt plasmid map with single-cut restriction enzyme sites. The expression vector ensures simultaneous synthesis of CMV-Ntt830-Feld12 and unmodified CMVNtt830.

FIG. 12A: SDS-PAGE gel of purification of mosaic VLPs comprising CMV-Ntt830-Feld12 and unmodified CMV-Ntt830 proteins, i.e. CMV-M-Fel. M—protein size marker PageRuler (Thermo Fisher Scientific, #26620); 0—total proteins in E. coli C2566/pET28-CMVxFeld1-CMVtt cells before induction; T—total proteins in E. coli C2566/pET28-CMVxFeld1-CMVtt cells after 18 h cultivation at 20° C. S—soluble proteins in cell extract before sucrose gradient centrifugation; P—insoluble proteins; 1-6—sucrose gradient fractions (from 60% at the bottom of tube to 0% at the top). The asterisc (*) denotes the relative position of CMV-Ntt830-Feld12 chimeric CMV polypeptide in gel.

FIG. 12B: SDS-PAGE gel analysis of purification of mosaic VLPs comprising CMV-Ntt830-Feld12 and unmodified CMV-Ntt830 proteins, i.e. CMV-M-Fel. M—protein size marker PageRuler (Thermo Fisher Scientific, #26620), 1—purified mosaic VLPs comprising CMV-Ntt830-Feld12 and unmodified CMV-Ntt830 proteins, i.e. CMV-M-Fel.

FIG. 12C: Electron microscopy analysis of purification of mosaic VLPs comprising CMV-Ntt830-Feld12 and unmodified CMV-Ntt830 proteins, i.e. CMV-M-Fel. Electron microscopy image of purified mosaic VLPs comprising CMV-Ntt830-Feld12 and unmodified CMV-Ntt830 proteins, i.e. CMV-M-Fel. White horizontal bar corresponds to 100 nm.

FIG. 13: CMV-M-Fel induces potent antibody responses against Fel d1. IgG against Fel d1 is shown as absorbance at 450 nm 14 days after immunization of naïve mice with Fel d1, VLP of CMV-Ntt830-Feld12 and mosaic VLPs comprising CMV-Ntt830-Feld12 and unmodified CMV-Ntt830 proteins, i.e. CMV-M-Fel.

FIG. 14A: Experimental design to investigate the protective effect of vaccination with CMV-M-Fel against allergic systemic and local reaction.

FIG. 14B: CMV-M-Fel induces protection against systemic challenge with Fel d1. Challenge was performed i.v with 3 microgram of Fel d1 extract, recombinant dimeric Fel d1.

FIG. 15: Description of pET28-CMVB2x19nanp-CMVtt plasmid map. The plasmid serves for expression of mosaic VLPs comprising CMV-Ntt830-19NANP and unmodified CMV-Ntt830 proteins, i.e. CMV-M-CSP.

FIG. 16: SDS-PAGE gel analysis of purification of mosaic VLPs comprising CMV-Ntt830-19NANP and unmodified CMV-Ntt830 proteins, i.e. CMV-M-CSP. M—protein size marker PageRuler (Thermo Fisher Scientific, #26620); S—soluble proteins in E. coli C2566 after 18 h cultivation at 20° C. in cell extract before sucrose gradient; P—insoluble proteins; 1-6—sucrose gradient fractions (from 60% at the bottom of tube to 0% at the top). The asterisc (*) denotes the relative position of CMV-Ntt830-19nanp in SDS/PAGE gel.

FIG. 17: Electron microscopy image of purified CMV-M-CSP mosaic VLPs. White horizontal bar corresponds to 100 nm.

FIG. 18: Description of pET-CMV-Ntt830-egy plasmid clone containing CMVNtt830 gene with cloned alpha-synuclein epitope cDNA. This plasmid close description is exemplarily for the description of plasmid clones pET-CMV-Ntt830-egy, pET-CMV-Ntt830-kne and pET-CMV-Ntt830-mdv which all have essentially the same sequences and gene organisations; they differ only in nucleotide sequences of coding for alpha-suynuclein peptide variants.

FIG. 19A: SDS-PAGE gel analysis of the purification of the VLP derived from the expression of CMV-Ntt830-egy. M—protein size marker PageRuler (Thermo Fisher Scientific, #26620); T—total proteins in E. coli C2566 cells after 18 h cultivation at 20° C.; 1-6—sucrose gradient fractions (from 60% at the bottom of tube to 0% at the top).

FIG. 19B: SDS-PAGE gel analysis of the purification of the VLP derived from the expression of CMV-Ntt830-kne. M—protein size marker PageRuler (Thermo Fisher Scientific, #26620); T—total proteins in E. coli C2566 cells after 18 h cultivation at 20° C.; 1-6—sucrose gradient fractions (from 60% at the bottom of tube to 0% at the top).

FIG. 19C: SDS-PAGE gel analysis of the purification of the VLP derived from the expression of CMV-Ntt830-mdv. M—protein size marker PageRuler (Thermo Fisher Scientific, #26620); T—total proteins in E. coli C2566 cells after 18 h cultivation at 20° C.; 1-6—sucrose gradient fractions (from 60% at the bottom of tube to 0% at the top).

FIG. 20A: SDS-PAGE gel analysis of purified CMV-Ntt830 and alpha-synuclein peptide fusion VLPs. M—protein size marker PageRuler (Thermo Fisher Scientific, #26620); 1—purified CMV-Ntt830B-mdv fusion protein VLPs; 2—purified CMV-Ntt830B-egy fusion protein VLPs; 3—purified CMV-Ntt830B-kne fusion protein VLPs; 4—purified CMV-Ntt830 unmodified VLPs.

FIG. 20B: Electron microscopy image of purified CMV-Ntt830B-egy fusion protein VLPs. White horizontal bar corresponds to 100 nm.

FIG. 20C: Electron microscopy image of purified CMV-Ntt830B-kne fusion protein VLPs. White horizontal bar corresponds to 100 nm.

FIG. 20D: Electron microscopy image of purified CMV-Ntt830B-kne fusion protein VLPs. White horizontal bar corresponds to 100 nm.

FIG. 21A: Description of pETDu-CMVB3d-CMVB3d-flIL5-CMVtt plasmid map. The plasmid serves for expression of mosaic VLPs comprising CMV-Ntt830-fel-IL-5 and unmodified CMV-Ntt830 proteins, i.e. CMV-M-fel-IL-5.

FIG. 21B: Description of pETDu-CMVB3d-CMVB3-flIL5-CMVtt plasmid map. The plasmid serves for expression of mosaic VLPs comprising CMV-Ntt830-fel-IL-5* and unmodified CMV-Ntt830 proteins, i.e. CMV-M-fel-IL-5*.

FIG. 22A: SDS-PAGE gel analysis of purification of mosaic VLPs comprising CMV-Ntt830-fel-IL-5 and unmodified CMV-Ntt830 proteins, i.e. CMV-M-fel-IL-5. M—protein size marker PageRuler (Thermo Fisher Scientific, #26620); 0—proteins in E. coli C2566/pETDu-CMVB3d-flIL5-CMVtt cells before IPTG induction; T—total proteins in E. coli C2566/pETDu-CMVB3d-flIL5-CMVtt cells after 18 h cultivation at 20° C. S—soluble proteins in cell extract before sucrose gradient (20-60%); P—insoluble proteins; 1-6—sucrose gradient fractions (from 60% at the bottom of tube to 0% at the top). The asterisc (*) denotes the relative position of CMV-Ntt830-fel-IL-5 in the SDS/PAGE gel.

FIG. 22B: SDS-PAGE gel analysis of purification of mosaic VLPs comprising CMV-Ntt830-fel-IL-5 and unmodified CMV-Ntt830 proteins, i.e. CMV-M-fel-IL-5*.

M—protein size marker PageRuler (Thermo Fisher Scientific, #26620); 0—proteins in E. coli C2566/pETDu-CMVB3-flIL5-CMVtt cells before IPTG induction; T—total proteins in E. coli C2566/pETDu-CMVB3-flIL5-CMVtt cells after 18 h cultivation at 20° C. S—soluble proteins in cell extract before sucrose gradient (20-60%); P—insoluble proteins; 1-6—sucrose gradient fractions (from 60% at the bottom of tube to 0% at the top). The asterisc (*) denotes the relative position of CMV-Ntt830-fel-IL-5* in the SDS/PAGE gel.

FIG. 23A: SDS-PAGE gel analysis of purified CMV-M-fel-IL-5 mosaic VLPs. M—protein size marker PageRuler (Thermo Fisher Scientific, #26620); 1—purified soluble CMV-M-fel-IL-5 mosaic VLPs after 13000 rpm clarification; 2—insoluble CMV-Ntt830-fel-IL-5/CMV-Ntt830 after 13000 rpm clarification.

FIG. 23B: Electron microscopy image of purified CMV-M-fel-IL-5 mosaic VLPs. Horizontal bar corresponds to 200 nm.

FIG. 23C: SDS-PAGE gel analysis of purified CMV-M-fel-IL-5* mosaic VLPs.

M—protein size marker PageRuler (Thermo Fisher Scientific, #26620); 1—purified soluble CMV-M-fel-IL-5* mosaic VLPs after 13000 rpm clarification.

FIG. 23D: Electron microscopy image of purified CMV-M-fel-IL-5* mosaic VLPs.

FIG. 24: Description of pETDu-CMVB3d-CMVB3d-2xfIIL5-CMVtt plasmid map. The plasmid serves for expression of mosaic VLPs comprising CMV-Ntt830-fel-IL-5 and unmodified CMV-Ntt830 proteins, i.e. CMV-M-2xfel-IL-5.

FIG. 25: SDS-PAGE gel analysis of purification of mosaic VLPs comprising CMV-Ntt830-2xfel-IL-5 and unmodified CMV-Ntt830 proteins, i.e. CMV-M-2xfel-IL-5. M—protein size marker PageRuler (Thermo Fisher Scientific, #26620); 0—proteins in E. coli C2566/pETDu-CMVB3d-2xflIL5-CMVtt cells before IPTG induction; T—total proteins in E. coli C2566/pETDu-CMVB3d-2xflIL5-CMVtt cells after 18 h cultivation at 20° C. S—soluble proteins in cell extract before sucrose gradient (20-60%); P—insoluble proteins; 1-6—sucrose gradient fractions (from 60% at the bottom of tube to 0% at the top). The asterisc (*) denotes the relative position of CMV-Ntt830-2xfel-IL-5 protein in the gel.

FIG. 26A: SDS-PAGE gel analysis of purified CMV-M-2xfel-IL-5 mosaic VLPs. M—protein size marker PageRuler (Thermo Fisher Scientific, #26620); 1—purified soluble CMV-M-2xfel-IL-5 mosaic VLPs after 13000 rpm clarification; 2—insoluble CMV-Ntt830-2xfel-IL-5/CMV-Ntt830 after 13000 rpm clarification.

FIG. 26B: Electron microscopy image of purified CMV-M-2xfel-IL-5 mosaic VLPs. Horizontal bar corresponds to 200 nm.

FIG. 27: Description of pETDu-CMVB3d-CMVB3d-cIL1b-CMVtt plasmid map. The plasmid serves for expression of mosaic VLPs comprising CMV-Ntt830-cIL-1b and unmodified CMV-Ntt830 proteins, i.e. CMV-M-cIL-1b.

FIG. 28: SDS-PAGE gel analysis of purification of mosaic VLPs comprising CMV-Ntt830-cIL-1b and unmodified CMV-Ntt830 proteins, i.e. CMV-M-cIL-1b. M—protein size marker PageRuler (Thermo Fisher Scientific, #26620); 0—proteins in E. coli C2566/pETDu-CMVB3d-cIL1b-CMVtt cells before IPTG induction; T—total proteins in E. coli C2566/pETDu-CMVB3d-cIL1b-CMVtt cells after 18 h cultivation at 20° C. S—soluble proteins in cell extract before sucrose gradient (20-60%); P—insoluble proteins; 1-6—sucrose gradient fractions (from 60% at the bottom of tube to 0% at the top). The asterisc (*) denotes the relative position of CMV-Ntt830-cIL-1b protein in the gel.

FIG. 29A: SDS-PAGE gel analysis of purified CMV-M-cIL-1b mosaic VLPs. M—protein size marker PageRuler (Thermo Fisher Scientific, #26620); 1—purified soluble CMV-M-cIL-1b mosaic VLPs after 13000 rpm clarification; 2—insoluble CMV-Ntt830-cIL-1b/CMV-Ntt830 after 13000 rpm clarification.

FIG. 29B: Electron microscopy image of purified CMV-M-cIL-1b mosaic VLPs. Horizontal bar corresponds to 200 nm.

### DETAILED DESCRIPTION OF THE INVENTION

Unless defined otherwise, all technical and scientific terms used herein have the same meanings as commonly understood by one of ordinary skill in the art to which this invention belongs. The herein described and disclosed embodiments, preferred embodiments and very preferred embodiments should apply to all aspects and other embodiments, preferred embodiments and very preferred embodiments irrespective of whether is specifically again referred to or its repetition is avoided for the sake of conciseness. The articles “a” and “an”, as used herein, refer to one or to more than one (i.e., to at least one) of the grammatical object of the article. The term “or”, as used herein, should be understood to mean “and/or”, unless the context clearly indicates otherwise.

Virus-like particle (VLP): The term “virus-like particle (VLP)” as used herein, refers to a non-replicative or non-infectious, preferably a non-replicative and non-infectious virus particle, or refers to a non-replicative or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering the host cell. A virus-like particle in accordance with the invention is non-replicative and non-infectious since it lacks all or part of the viral genome or genome function. A virus-like particle in accordance with the invention may contain nucleic acid distinct from their genome. Recombinantly produced virus-like particles typically contain host cell derived RNA. A typical and preferred embodiment of a virus-like particle in accordance with the present invention is a viral capsid composed of polypeptides of the invention. A virus-like particle is typically a macromolecular assembly composed of viral coat protein which typically comprises 60, 120, 180, 240, 300, 360, or more than 360 protein subunits per virus-like particle. Typically and preferably, the interactions of these subunits lead to the formation of viral capsid or viral-capsid like structure with an inherent repetitive organization. One feature of a virus-like particle is its highly ordered and repetitive arrangement of its subunits.

Modified virus-like particle of CMV: The term “modified virus-like particle of CMV” refers to a virus-like particle comprising at least one fusion protein which comprises a CMV polypeptide. Typically and preferably, modified virus-like particles of CMV resemble the structure of the capsid of CMV. Modified virus-like particles of CMV are non-replicative and/or non-infectious, and lack at least the gene or genes encoding for the replication machinery of the CMV, and typically also lack the gene or genes encoding the protein or proteins responsible for viral attachment to or entry into the host. This definition includes also modified virus-like particles in which the aforementioned gene or genes are still present but inactive. Preferably, non-replicative and/or non-infectious modified virus-like particles are obtained by recombinant gene technology and typically and preferably do not comprise the viral genome. Modified virus-like particles comprising two or more different polypeptides are referred to as “mosaic VLPs”, and are in particular encompassed by the invention. Mosaic modified virus-like particle are very preferred embodiments and aspects of the present invention. Thus, in one embodiment, the modified virus-like particle according to the invention comprises at least two different species of polypeptides, very preferably said mosaic VLPs comprise two different species of CMV polypeptides optionally modified in accordance with the present invention leading to mosaic modified CMC VLPs. Preferably, a modified VLP of CMV is a macromolecular assembly composed of CMV polypeptides modified in accordance with the present invention typically comprising 180 of such protein subunits per VLP.

Polypeptide: The term “polypeptide” as used herein refers to a polymer composed of amino acid monomers which are linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. The term “polypeptide” as used herein should also refer, typically and preferably to a polypeptide as defined before and encompassing modifications such as post-translational modifications, including but not limited to glycosylations. In a preferred embodiment, said term “polypeptide” as used herein should refer to a polypeptide as defined before and not encompassing modifications such as post-translational modifications such as glycosylations. In particular, for said biologically active peptides, said modifications such as said glycosylations can occur even in vivo thereafter, for example, by bacteria.

Cucumber Mosaic Virus (CMV) polypeptide, CMV polypeptide: The term “cucumber mosaic virus (CMV) polypeptide” as used herein refers to a polypeptide comprising or preferably consisting of: (i) an amino acid sequence of a coat protein of cucumber mosaic virus (CMV), or (ii) a mutated amino acid sequence, wherein the amino acid sequence to be mutated is an amino acid sequence of a coat protein of CMV, and wherein said mutated amino acid sequence and said amino acid sequence to be mutated, i.e. said coat protein of CMV, show a sequence identity of at least 90%, preferably of at least 95%, further preferably of at least 98% and again more preferably of at least 99%. Typically and preferably, the CMV polypeptide is capable of forming a virus-like particle of CMV upon expression by self-assembly. As used herein, the term “chimeric” is intended—when referring in the context of polypeptides—to refer to polypeptides comprising polypeptidic components from two or more distinct sources. This term is further intended to confer to the specific manner in which the polypeptide components are bound or attached together, namely by way of fusion and peptide bonds, respectively. The term “chimeric CMV polypeptide” is thus defined as such and in particular in accordance with the present invention.

Coat protein (CP) of cucumber mosaic virus (CMV): The term “coat protein (CP) of cucumber mosaic virus (CMV)”, as used herein, refers to a coat protein of the cucumber mosaic virus which occurs in nature. Due to extremely wide host range of the cucumber mosaic virus, a lot of different strains and isolates of CMV are known and the sequences of the coat proteins of said strains and isolates have been determined and are, thus, known to the skilled person in the art as well. The sequences of said coat proteins (CPs) of CMV are described in and retrievable from the known databases such as Genbank, www.dpvweb.net, or www.ncbi.nlm.nih.gov/protein/. Specific examples CPs of CMV are described in WO 2016/062720 at page 12, line 8 to page 13, line 25, the disclosure of which are explicitly incorporated herein by way of reference. A very preferred example and embodiment of a CMV coat protein is provided in SEQ ID NO:62. Thus, preferably, the term “coat protein of cucumber mosaic virus (CMV)”, as used herein, refers to an amino acid sequence of a coat protein of CMV, wherein said amino acid sequence comprises, or preferably consists of, SEQ ID NO:62 or an amino acid sequence having a sequence identity of at least 75%, preferably of at least 80%, more preferably of at least 85%, again further preferably of at least 90%, again more preferably of at least 95%, still further preferably of at least 98% and still again further more preferably of at least 99% of SEQ ID NO:62.

It is noteworthy that these strains and isolates have highly similar coat protein sequences at different protein domains, including the N-terminus of the coat protein. In particular, 98.1% of all completely sequenced CMV isolates share more than 85% sequence identity within the first 28 amino acids of their coat protein sequence, and still 79.5% of all completely sequenced CMV isolates share more than 90% sequence identity within the first 28 amino acids of their coat protein sequence.

N-terminal region of the CMV polypeptide: The term “N-terminal region of the CMV polypeptide” as used herein, refers either to the N-terminus of said CMV polypeptide, and in particular to the N-terminus of a coat protein of CMV, or to the region of the N-terminus of said CMV polypeptide or said coat protein of CMV but starting with the second amino acid of the N-terminus of said CMV polypeptide or said coat protein of CMV if said CMV polypeptide or said coat protein comprises a N-terminal methionine residue. Preferably, in case said CMV polypeptide or said coat protein comprises a N-terminal methionine residue, from a practical point of view, the start-codon encoding methionine will usually be deleted and added to the N-terminus of the Th cell epitope in accordance with the present invention. Further preferably, one, two or three additional amino acids, preferably one amino acid, may be optionally inserted between the stating methionine and the Th cell epitope for cloning purposes. The term “N-terminal region of the mutated amino acid sequence of a CMV polypeptide or a CMV coat protein” as used herein, refers either to the N-terminus of said mutated amino acid sequence of said CMV polypeptide or said coat protein of CMV, or to the region of the N-terminus of said mutated amino acid sequence of said CMV polypeptide or said coat protein of CMV but starting with the second amino acid of the N-terminus of said mutated amino acid sequence of said CMV polypeptide or said coat protein of CMV if said mutated amino acid sequence comprises a N-terminal methionine residue. Preferably, in case said CMV polypeptide or said coat protein comprises a N-terminal methionine residue, from a practical point of view, the start-codon encoding methionine will usually be deleted and added to the N-terminus of the Th cell epitope. Further preferably, one, two or three additional amino acids, preferably one amino acid, may be optionally inserted between the stating methionine and the Th cell epitope for cloning purposes.

Recombinant polypeptide: In the context of the invention the term “recombinant” when used in the context of a polypeptide refers to a polypeptide which is obtained by a process which comprises at least one step of recombinant DNA technology. Typically and preferably, a recombinant polypeptide is produced in a prokaryotic expression system. It is apparent for the artisan that recombinantly produced polypeptides which are expressed in a prokaryotic expression system such as E. coli may comprise an N-terminal methionine residue. The N-terminal methionine residue is typically cleaved off the recombinant polypeptide in the expression host during the maturation of the recombinant polypeptide. However, the cleavage of the N-terminal methionine may be incomplete. Thus, a preparation of a recombinant polypeptide may comprise a mixture of otherwise identical polypeptides with and without an N-terminal methionine residue. Typically and preferably, a preparation of a recombinant polypeptide comprises less than 10%, more preferably less than 5%, and still more preferably less than 1% recombinant polypeptide with an N-terminal methionine residue.

Recombinant modified virus-like particle: In the context of the invention the term “recombinant modified virus-like particle” refers to a modified virus-like particle (VLP) which is obtained by a process which comprises at least one step of recombinant DNA technology.

Mutated amino acid sequence: The term “mutated amino acid sequence” refers to an amino acid sequence which is obtained by introducing a defined set of mutations into an amino acid sequence to be mutated. In the context of the invention, said amino acid sequence to be mutated typically and preferably is an amino acid sequence of a coat protein of CMV. Thus, a mutated amino acid sequence differs from an amino acid sequence of a coat protein of CMV in at least one amino acid residue, wherein said mutated amino acid sequence and said amino acid sequence to be mutated show a sequence identity of at least 90%. Typically and preferably said mutated amino acid sequence and said amino acid sequence to be mutated show a sequence identity of at least 91%, 92%, 93% 94%, 95%, 96%, 97%, 98%, or 99%. Preferably, said mutated amino acid sequence and said sequence to be mutated differ in at most 11, 10, 9, 8, 7, 6, 4, 3, 2, or 1 amino acid residues, wherein further preferably said difference is selected from insertion, deletion and amino acid exchange. Preferably, the mutated amino acid sequence differs from an amino acid sequence of a coat protein of CMV in least one amino acid, wherein preferably said difference is an amino acid exchange.

Position corresponding to residues . . . : The position on an amino acid sequence, which is corresponding to given residues of another amino acid sequence can be identified by sequence alignment, typically and preferably by using the BLASTP algorithm, most preferably using the standard settings. Typical and preferred standard settings are: expect threshold: 10; word size: 3; max matches in a query range: 0; matrix: BLOSUM62; gap costs: existence 11, extension 1; compositional adjustments: conditional compositional score matrix adjustment.

Sequence identity: The sequence identity of two given amino acid sequences is determined based on an alignment of both sequences. Algorithms for the determination of sequence identity are available to the artisan. Preferably, the sequence identity of two amino acid sequences is determined using publicly available computer homology programs such as the “BLAST” program (http://blast.ncbi.nlm.nih.gov/Blast.cgi) or the “CLUSTALW” (http://www.genome.jp/tools/clustalw/), and hereby preferably by the “BLAST” program provided on the NCBI homepage at http://blast.ncbi.nlm.nih.gov/Blast.cgi, using the default settings provided therein. Typical and preferred standard settings are: expect threshold: 10; word size: 3; max matches in a query range: 0; matrix: BLOSUM62; gap costs: existence 11, extension 1; compositional adjustments: conditional compositional score matrix adjustment.

Amino acid exchange: The term amino acid exchange refers to the exchange of a given amino acid residue in an amino acid sequence by any other amino acid residue having a different chemical structure, preferably by another proteinogenic amino acid residue. Thus, in contrast to insertion or deletion of an amino acid, the amino acid exchange does not change the total number of amino acids of said amino acid sequence.

Epitope: The term epitope refers to continuous or discontinuous portions of an antigen, preferably a polypeptide, wherein said portions can be specifically bound by an antibody or by a T-cell receptor within the context of an MEW molecule. With respect to antibodies, specific binding excludes non-specific binding but does not necessarily exclude cross-reactivity. An epitope typically comprise 5-20 amino acids in a spatial conformation which is unique to the antigenic site.

T helper (Th) cell epitope: The term “T helper (Th) cell epitope” as used herein refers to an epitope that is capable of recognition by a helper Th cell. Typically and preferably, the term “Th cell epitope” as used herein refers to a Th cell epitope that is capable of binding to at least one, preferably more than one MEW class II molecules. The simplest way to determine whether a peptide sequence is a Th cell epitope is to measure the ability of the peptide to bind to individual MEW class II molecules. This may be measured by the ability of the peptide to compete with the binding of a known Th cell epitope peptide to the MEW class II molecule. A representative selection of HLA-DR molecules are described in e.g. Alexander J, et al., Immunity (1994) 1:751-761. Affinities of Th cell epitopes for MEW class II molecules should be at least 10⁻⁵M. A representative collection of MEW class II molecules present in different individuals is given in Panina-Bordignon P, et al., Eur J Immunol (1989) 19:2237-2242. As a consequence, the term “Th cell epitope” as used herein preferably refers to a Th cell epitope that generates a measurable T cell response upon immunization and boosting. Moreover, and again further preferred, the term “Th cell epitope” as used herein preferably refers to a Th cell epitope that is capable of binding to at least one, preferably to at least two, and even more preferably to at least three DR alleles selected from of DR1, DR2w2b, DR3, DR4w4, DR4w14, DR5, DR7, DR52a, DRw53, DR2w2a; and preferably selected from DR1, DR2w2b, DR4w4, DR4w14, DR5, DR7, DRw53, DR2w2a, with an affinity at least 500 nM (as described in Alexander J, et al., Immunity (1994) 1:751-761 and references cited herein); a preferred binding assay to evaluate said affinities is the one described by Sette A, et al., J Immunol (1989) 142:35-40. In an even again more preferable manner, the term “Th cell epitope” as used herein refers to a Th cell epitope that is capable of binding to at least one, preferably to at least two, and even more preferably to at least three DR alleles selected from DR1, DR2w2b, DR4w4, DR4w14, DR5, DR7, DRw53, DR2w2a, with an affinity at least 500 nM (as described in Alexander J, et al., Immunity (1994) 1:751-761 and references cited herein); a preferred binding assay to evaluate said affinities is the one described by Sette A, et al., J Immunol (1989) 142:35-40. Th cell epitopes are described, and known to the skilled person in the art, such as by Alexander J, et al., Immunity (1994) 1:751-761, Panina-Bordignon P, et al., Eur J Immunol (1989) 19:2237-2242, Calvo-Calle J M, et al., J Immunol (1997) 159:1362-1373, and Valmori D, et al., J Immunol (1992) 149:717-721.

Antigenic polypeptide: As used herein, the term “antigenic polypeptide” refers to a molecule capable of being bound by an antibody or a T-cell receptor (TCR) if presented by MEW molecules. An antigenic polypeptide is additionally capable of being recognized by the immune system and/or being capable of inducing a humoral immune response and/or cellular immune response leading to the activation of B- and/or T-lymphocytes. An antigenic polypeptide can have one or more epitopes (B- and T-epitopes). Antigenic polypeptides as used herein may also be mixtures of several individual antigenic polypeptides. The polypeptides of the invention, in particular said inventive fusion proteins which are forming the inventive modified virus-like particles, comprise the antigenic polypeptide.

Peanut Allergen: The term “peanut allergen”, as used herein, refers to any protein of the Arachis hypogaea species, and isoforms thereof, suggested to cause an allergy for a human. Preferably, the term “peanut allergen”, as used herein, refers to any of the suggested peanut allergens, and isoforms thereof, as retrievable under www.allergen.org or a protein with an amino acid sequence of at least 90%, preferably of at least 92%, further preferably of at least 95%, and again further preferably of at least 98% amino acid sequence identity with such a peanut allergen and isoform thereof. More preferably, the term “peanut allergen”, as used herein, refers to any of the suggested currently 17 peanut allergens, and isoforms thereof, as retrievable under www.allergen.org or a protein with an amino acid sequence of at least 90%, preferably of at least 92%, further preferably of at least 95%, and again further preferably of at least 98% amino acid sequence identity with such a peanut allergen and isoform thereof. Again more preferably, the term “peanut allergen”, as used herein, refers to any one of the peanut allergens, and isoforms thereof, selected from Ara h1, Ara h2, Ara h3, Ara h4, Ara h5, Ara h6, Ara h7, Ara h8, Ara h9, Ara h10, Ara h11, Ara h12, Ara h13, Ara h14, Ara h15, Ara h16 and Ara h17, or a protein with an amino acid sequence of at least 90%, preferably of at least 92%, further preferably of at least 95%, and again further preferably of at least 98% amino acid sequence identity with such a peanut allergen and isoform thereof. Again more preferably, the term “peanut allergen”, as used herein, refers to any one of the peanut allergens and isoforms thereof, selected from Ara h1, Ara h2, Ara h3, and Ara h6 or a protein with an amino acid sequence of at least 90%, preferably of at least 92%, further preferably of at least 95%, and again further preferably of at least 98% amino acid sequence identity with such a peanut allergen and isoform thereof. Again more preferably, the term “peanut allergen”, as used herein, refers to any proteins selected from Ara h1, Ara h2, Ara h3 and Ara h6, and isoforms thereof, or a protein with an amino acid sequence of at least 90%, preferably of at least 92%, further preferably of at least 95%, and again further preferably of at least 98% amino acid sequence identity with such a peanut allergen. Again more preferably, the term “peanut allergen”, as used herein, refers to any proteins selected from Ara h1, Ara h2, Ara h201, Ara h202, Ara h3 and Ara h6, or a protein with an amino acid sequence of at least 90%, preferably of at least 92%, further preferably of at least 95%, and again further preferably of at least 98% amino acid sequence identity with such a peanut allergen.

Fel d1 protein: The term “Fel d1 protein”, as used herein, refers to a protein comprising or alternatively consisting of chain 1 of Fel d1 and chain 2 of Fel d1. Preferably chain 1 of Fel d1 and chain 2 of Fel d1 are linked covalently. In one preferred embodiment, the chain 1 of Fel d1 and chain 2 of Fel d1 are linked via at least one disulfide bond. In another preferred embodiment, the chain 1 and chain 2 are fused either directly or via a spacer, in which case said Fel d1 protein further comprises or alternatively consists of a spacer. Preferably the Fel d1 protein, as defined herein, consists of at most 300, even more preferably at most 200 amino acids in total. Typically and preferably, Fel d1 protein, according to the invention, is capable of inducing in vivo the production of antibody specifically binding to either the naturally occurring Fel d1.

Chain 1 of Fel d1: The term “chain 1 of Fel d1”, as used herein, refers to a polypeptide comprising or alternatively consisting of an amino acid sequence as of SEQ ID NO:76 or a homologous sequence thereof. The term “homologous sequence of SEQ ID NO:76”, as used herein, refers to a polypeptide that has an identity to SEQ ID NO:76 which is greater than 80%, more preferably greater than 90%, and even more preferably greater than 95%. The term “chain 1 of Fel d1”, as used herein, should also refer to a polypeptide encompassing at least one post-translational modification, including but not limited to at least one glycosylation, of chain 1 of Fel d1, as defined herein. Preferably the chain 1 of Fel d1, as defined herein, consists of at most 130, even more preferably at most 100 amino acids in total.

Chain 2 of Fel d1: The term “chain 2 of Fel d1”, as used herein, refers to a polypeptide comprising or alternatively consisting of an amino acid sequence as of SEQ ID NO:77, SEQ ID NO:78 or SEQ ID NO:79, or a homologous sequence thereof. The term “homologous sequence of SEQ ID NO:77, SEQ ID NO:78 or SEQ ID NO:79, as used herein, refers to a polypeptide that has an identity to SEQ ID NO:77, SEQ ID NO:78 or SEQ ID NO:79 which is greater than 80%, more preferably greater than 90%, and even more preferably greater than 95%. The term “chain 2 of Fel d1”, as used herein, should also refer to a polypeptide encompassing at least one post-translational modification, including but not limited to at least one glycosylation, of chain 2 of Fel d1, as defined herein Preferably the chain 2 of Fel d1, as defined herein, consists of at most 150, even more preferably at most 130, still more preferably at most 100 amino acids in total.

Adjuvant: The term “adjuvant” as used herein refers to non-specific stimulators of the immune response or substances that allow generation of a depot in the host which when combined with the vaccine and pharmaceutical composition, respectively, of the present invention may provide for an even more enhanced immune response. Preferred adjuvants are complete and incomplete Freund's adjuvant, aluminum containing adjuvant, preferably aluminum hydroxide, and modified muramyldipeptide. Further preferred adjuvants are mineral gels such as aluminum hydroxide, surface active substances such as lyso lecithin, pluronic polyols, polyanions, peptides, oil emulsions, keyhole limpet hemocyanins, dinitrophenol, and human adjuvants such as BCG (bacille Calmette Guerin) and Corynebacterium parvum. Such adjuvants are also well known in the art. Further adjuvants that can be administered with the compositions of the invention include, but are not limited to, Monophosphoryl lipid immunomodulator, AdjuVax 100a, QS-21, QS-18, CRL1005, Aluminum salts (Alum), MF-59, OM-174, OM-197, OM-294, and Virosomal adjuvant technology. The adjuvants may also comprise mixtures of these substances. Virus-like particles have been generally described as an adjuvant. However, the term “adjuvant”, as used within the context of this application, refers to an adjuvant not being the inventive modified virus-like particle. Rather “adjuvant” relates to an additional, distinct component of the inventive compositions, vaccines or pharmaceutical compositions.

Amino acid linker: The term “amino acid linker” as used herein, refers to a linker consisting exclusively of amino acid residues. The amino acid residues of the amino acid linker are composed of naturally occurring amino acids or unnatural amino acids known in the art, all-L or all-D or mixtures thereof. The amino acid residues of the amino acid linker are preferably naturally occurring amino acids, all-L or all-D or mixtures thereof.

GS-linker: The term “GS-linker”, as used herein refers to a linker solely consisting of glycine and serine amino acid residues. The GS-linker in accordance with the present invention comprise at least one glycine and at least one serine residue. Typically and preferably, the GS-linker in accordance with the present invention has a length of at most 50 amino acids, and typically and further preferably, the GS-linker in accordance with the present invention has a length of at most 30 amino acids.

GST-linker: The term “GST-linker”, as used herein refers to a linker comprising, preferably consisting of, glycine, serine and threonine amino acid residues. The GST-linker in accordance with the present invention comprise at least one glycine, at least one serine and at least one threonine residue. Typically and preferably, the GST-linker in accordance with the present invention has a length of at most 50 amino acids, and typically and further preferably, the GST-linker in accordance with the present invention has a length of at most 30 amino acids.

GSED-linker: The term “GSED-linker”, as used herein refers to a linker comprising, preferably consisting of, glycine, serine, glutamic acid and aspartic acid amino acid residues. The GSED-linker in accordance with the present invention comprise at least one glycine, at least one serine, at least one glutamic acid and at least one aspartic acid residue. Typically and preferably, the GSED-linker in accordance with the present invention has a length of at most 50 amino acids, and typically and further preferably, the GSED-linker in accordance with the present invention has a length of at most 30 amino acids.

Immunostimulatory substance: As used herein, the term “immunostimulatory substance” refers to a substance capable of inducing and/or enhancing an immune response. Immunostimulatory substances, as used herein, include, but are not limited to, toll-like receptor activating substances and substances inducing cytokine secretion. Toll-like receptor activating substances include, but are not limited to, immunostimulatory nucleic acids, peptideoglycans, lipopolysaccharides, lipoteichonic acids, imidazoquinoline compounds, flagellins, lipoproteins, and immuno stimulatory organic substances such as taxol.

Immunostimulatory nucleic acid (ISS-NA): As used herein, the term immunostimulatory nucleic acid refers to a nucleic acid capable of inducing and/or enhancing an immune response. Immunostimulatory nucleic acids comprise ribonucleic acids and in particular desoxyribonucleic acids, wherein both, ribonucleic acids and desoxyribonucleic acids may be either double stranded or single stranded. Preferred ISS-NA are desoxyribonucleic acids, wherein further preferably said desoxyribonucleic acids are single stranded. Preferably, immunostimulatory nucleic acids contain at least one CpG motif comprising an unmethylated C. Very preferred immunostimulatory nucleic acids comprise at least one CpG motif, wherein said at least one CpG motif comprises or preferably consist of at least one, preferably one, CG dinucleotide, wherein the C is unmethylated. Preferably, but not necessarily, said CG dinucleotide is part of a palindromic sequence. The term immunostimulatory nucleic acid also refers to nucleic acids that contain modified bases, preferably 4-bromo-cytosine. Specifically preferred in the context of the invention are ISS-NA which are capable of stimulating IFN-alpha production in dendritic cells. Immunostimulatory nucleic acids useful for the purpose of the invention are described, for example, in WO2007/068747A1.

Oligonucleotide: As used herein, the term “oligonucleotide” refers to a nucleic acid sequence comprising 2 or more nucleotides, preferably about 6 to about 200 nucleotides, and more preferably 20 to about 100 nucleotides, and most preferably 20 to 40 nucleotides. Very preferably, oligonucleotides comprise about 30 nucleotides, more preferably oligonucleotides comprise exactly 30 nucleotides, and most preferably oligonucleotides consist of exactly 30 nucleotides. Oligonucleotides are polyribonucleotides or polydeoxribonucleotides and are preferably selected from (a) unmodified RNA or DNA, and (b) modified RNA or DNA. The modification may comprise the backbone or nucleotide analogues. Oligonucleotides are preferably selected from the group consisting of (a) single- and double-stranded DNA, (b) DNA that is a mixture of single- and double-stranded regions, (c) single- and double-stranded RNA, (d) RNA that is mixture of single- and double-stranded regions, and (e) hybrid molecules comprising DNA and RNA that are single-stranded or, more preferably, double-stranded or a mixture of single- and double-stranded regions. Preferred nucleotide modifications/analogs are selected from the group consisting of (a) peptide nucleic acid, (b) inosin, (c) tritylated bases, (d) phosphorothioates, (e) alkylphosphorothioates, (f) 5-nitroindole desoxyribofliranosyl, (g) 5-methyldesoxycytosine, and (h) 5,6-dihydro-5,6-dihydroxydesoxythymidine. Phosphothioated nucleotides are protected against degradation in a cell or an organism and are therefore preferred nucleotide modifications. Unmodified oligonucleotides consisting exclusively of phosphodiester bound nucleotides, typically are more active than modified nucleotides and are therefore generally preferred in the context of the invention. Most preferred are oligonucleotides consisting exclusively of phosphodiester bound deoxinucleo tides, wherein further preferably said oligonucleotides are single stranded. Further preferred are oligonucleotides capable of stimulating IFN-alpha production in cells, preferably in dendritic cells. Very preferred oligonucleotides capable of stimulating IFN-alpha production in cells are selected from A-type CpGs and C-type CpGs. Further preferred are RNA-molecules without a Cap.

CpG motif: As used herein, the term “CpG motif refers to a pattern of nucleotides that includes an unmethylated central CpG, i.e. the unmethylated CpG dinucleotide, in which the C is unmethylated, surrounded by at least one base, preferably one or two nucleotides, flanking (on the 3′ and the 5′ side of) the central CpG. Typically and preferably, the CpG motif as used herein, comprises or alternatively consists of the unmethylated CpG dinucleotide and two nucleotides on its 5 ‘ and 3’ ends. Without being bound by theory, the bases flanking the CpG confer a significant part of the activity to the CpG oligonucleotide.

Unmethylated CpG-containing oligonucleotide: As used herein, the term “unmethylated CpG-containing oligonucleotide” or “CpG” refers to an oligonucleotide, preferably to an oligodesoxynucleotide, containing at least one CpG motif. Thus, a CpG contains at least one unmethylated cytosine, guanine dinucleotide. Preferred CpGs stimulate/activate, e.g. have a mitogenic effect on, or induce or increase cytokine expression by, a vertebrate bone marrow derived cell. For example, CpGs can be useful in activating B cells, NK cells and antigen-presenting cells, such as dendritic cells, monocytes and macrophages. Preferably, CpG relates to an oligodesoxynucleotide, preferably to a single stranded oligodesoxynucleotide, containing an unmethylated cytosine followed 3′ by a guanosine, wherein said unmethylated cytosine and said guanosine are linked by a phosphate bond, wherein preferably said phosphate bound is a phosphodiester bound or a phosphothioate bound, and wherein further preferably said phosphate bond is a phosphodiester bound. CpGs can include nucleotide analogs such as analogs containing phosphorothio ester bonds and can be double-stranded or single-stranded. Generally, double-stranded molecules are more stable in vivo, while single-stranded molecules have increased immune activity. Preferably, as used herein, a CpG is an oligonucleotide that is at least about ten nucleotides in length and comprises at least one CpG motif, wherein further preferably said CpG is 10 to 60, more preferably 15 to 50, still more preferably 20 to 40, still more preferably about 30, and most preferably exactly 30 nucleotides in length. A CpG may consist of methylated and/or unmethylated nucleotides, wherein said at least one CpG motif comprises at least one CG dinucleotide wherein the C is unmethylated. The CpG may also comprise methylated and unmethylated sequence stretches, wherein said at least one CpG motif comprises at least one CG dinucleotide wherein the C is unmethylated. Very preferably, CpG relates to a single stranded oligodesoxynucleotide containing an unmethylated cytosine followed 3′ by a guanosine, wherein said unmethylated cytosine and said guanosine are linked by a phosphodiester bound. The CpGs can include nucleotide analogs such as analogs containing phosphorothioester bonds and can be double-stranded or single-stranded. Generally, phosphodiester CpGs are A-type CpGs as indicated below, while phosphothioester stabilized CpGs are B-type CpGs. Preferred CpG oligonucleotides in the context of the invention are A-type CpGs.

A-type CpG: As used herein, the term “A-type CpG” or “D-type CpG” refers to an oligodesoxynucleotide (ODN) comprising at least one CpG motif. A-type CpGs preferentially stimulate activation of T cells and the maturation of dendritic cells and are capable of stimulating IFN-alpha production. In A-type CpGs, the nucleotides of the at least one CpG motif are linked by at least one phosphodiester bond. A-type CpGs comprise at least one phosphodiester bond CpG motif which may be flanked at its 5′ end and/or, preferably and, at its 3′ end by phosphorothioate bound nucleotides. Preferably, the CpG motif, and hereby preferably the CG dinucleotide and its immediate flanking regions comprising at least one, preferably two nucleotides, are composed of phosphodiester nucleotides. Preferred A-type CpGs exclusively consist of phosphodiester (PO) bond nucleotides. Typically and preferably, the poly G motif comprises or alternatively consists of at least one, preferably at least three, at least 4, 5, 6, 7, 8, 9, 10, 11, 12, 13, 14, or 15 Gs (guanosines), most preferably by at least 10 Gs. Preferably, the A-type CpG of the invention comprises or alternatively consists of a palindromic sequence.

Packaged: The term “packaged” as used herein refers to the state of a polyanionic macromolecule or immunostimulatory substances in relation to the core particle and VLP, respectively. The term “packaged” as used herein includes binding that may be covalent, e.g., by chemically coupling, or non-covalent, e.g., ionic interactions, hydrophobic interactions, hydrogen bonds, etc. The term also includes the enclosement, or partial enclosement, of a polyanionic macromolecule. Thus, the polyanionic macromolecule or immunostimulatory substances can be enclosed by the VLP without the existence of an actual binding, in particular of a covalent binding. In preferred embodiments, the at least one polyanionic macromolecule or immunostimulatory substances is packaged inside the VLP, most preferably in a non-covalent manner. In case said immunostimulatory substances is nucleic acid, preferably a DNA, the term packaged implies that said nucleic acid is not accessible to nucleases hydrolysis, preferably not accessible to DNAse hydrolysis (e.g. DNasel or Benzonase), wherein preferably said accessibility is assayed as described in Examples 11-17 of WO2003/024481A2.

Effective amount: As used herein, the term “effective amount” refers to an amount necessary or sufficient to realize a desired biologic effect. An effective amount of the composition, or alternatively the pharmaceutical composition, would be the amount that achieves this selected result, and such an amount could be determined as a matter of routine by a person skilled in the art. Preferably, the term “effective amount”, as used herein, refers to an amount necessary or sufficient to be effective to reduce levels of said at least one peanut allergen to a level that causes the reduction of at least one symptom caused by the peanut allergy. Preferably, the term “effective amount”, as used herein, refers to an amount necessary or sufficient to be effective to neutralize the activity of at least one peanut allergen. The effective amount can vary depending on the particular composition being administered and the size of the subject. One of ordinary skill in the art can empirically determine the effective amount of a particular composition of the present invention without necessitating undue experimentation.

Treatment: As used herein, the terms “treatment”, “treat”, “treated” or “treating” refer to prophylaxis and/or therapy. In one embodiment, the terms “treatment”, “treat”, “treated” or “treating” refer to a therapeutic treatment. In another embodiment, the terms “treatment”, “treat”, “treated” or “treating” refer to a prophylactic treatment.

In a first aspect, the present invention provides modified virus-like particle (VLP) of cucumber mosaic virus (CMV) comprising at least one fusion protein, wherein said at least one fusion protein comprises, or preferably consists of, a) a chimeric CMV polypeptide, wherein said chimeric CMV polypeptide comprises, or preferably consists of (i) a CMV polypeptide, wherein said CMV polypeptide comprises, or preferably consists of, a coat protein of CMV, wherein preferably said coat protein of CMV comprises, or preferably consists of, SEQ ID NO:62; or an amino acid sequence having a sequence identity of at least 75%, preferably of at least 80%, more preferably of at least 85%, again further preferably of at least 90%, again more preferably of at least 95%, still further preferably of at least 98% and still again further more preferably of at least 99% with SEQ ID NO:62; and (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and (iii) a T helper cell epitope, wherein said T helper cell epitope replaces a N-terminal region of said CMV polypeptide, and wherein preferably said N-terminal region of said CMV polypeptide corresponds to amino acids 2-12 of SEQ ID NO:62. In a very preferred embodiment, said chimeric CMV polypeptide further comprises a first amino acid linker, preferably a first amino acid linker and a second amino acid linker, wherein said first amino acid linker is positioned at the N- or at the C-terminus of said antigenic polypeptide, and wherein said first amino acid linker is selected from the group consisting of: (a.) a polyglycine linker (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker comprising at least one Gly, at least one Ser, and at least one amino acid selected from Thr, Ala, Lys, Asp and Glu.

The preferred amino acid linkers, namely the GS-linkers or the GSED-linkers, have been found to be very beneficial to overcome spherical disorders hindering the assembly process forming the modified VLPs of the present invention and/or to overcome aggregation tendencies between formed modified VLPs of the present invention and/or to increase flexibility for insertion of even very long antigenic polypeptides. This is in particular the case if said GS-linkers or GSED-linkers are further applied as first amino acid linker and as second amino acid linker, and again further, if said first amino acid linker and said second amino acid linker mimic the amino acid sequence situation as present without said inserted antigenic polypeptide, This was in particular beneficial for the preferred CMV polypeptides of the present invention. Thus, mimicking the amino acid sequence situation as present without said inserted antigenic polypeptide has been found to be particularly beneficial for the insertion of the antigenic polypeptide between amino acid residues of said CMV polypeptide corresponding to amino acid residues of position 84 and position 85 of SEQ ID NO:62, and, in particular, between positions corresponding to Ser(88) and Tyr(89) of SEQ ID NO:5. The insertion of the antigenic polypeptide after GS (i.e. after position 84 of SEQ ID NO:62 and position 88 of SEQ ID NO:5, respectively) and before YY (i.e. before position 85 of SEQ ID NO:62 and position 89 of SEQ ID NO:5, respectively) by preferably using GS-linkers or GSED-linkers, and in particular by using a second amino acid linker positioned at the C-terminus of the antigenic polypeptide and being a GS-linker or a GSED-linker ending with a GS has been found to be particularly beneficial.

In a further aspect, the present invention provides a modified VLP of CMV comprising at least one fusion protein, wherein said at least one fusion protein comprises, or preferably consists of, a) a chimeric CMV polypeptide, wherein said chimeric CMV polypeptide comprises, or preferably consists of (i) a CMV polypeptide, wherein said CMV polypeptide comprises, or preferably consists of, a coat protein of CMV, wherein preferably said coat protein of CMV comprises, or preferably consists of, SEQ ID NO:62; or an amino acid sequence having a sequence identity of at least 75%, preferably of at least 80%, more preferably of at least 85%, again further preferably of at least 90%, again more preferably of at least 95%, still further preferably of at least 98% and still again further more preferably of at least 99% with said coat protein, preferably with said SEQ ID NO:62; and (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and (iii) a first amino acid linker, preferably a first amino acid linker and a second amino acid linker, wherein said first amino acid linker is positioned at the N- or at the C-terminus of said antigenic polypeptide, and wherein preferably said first amino acid linker is selected from the group consisting of: (a.) a polyglycine linker (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker comprising at least one Gly, at least one Ser, and at least one amino acid selected from Thr, Ala, Lys, Asp and Glu. In a very preferred embodiment, said chimeric CMV polypeptide further comprises a T helper cell epitope, wherein said T helper cell epitope replaces a N-terminal region of said CMV polypeptide, and wherein preferably said N-terminal region of said CMV polypeptide corresponds to amino acids 2-12 of SEQ ID NO:62.

In another aspect, the present invention provides a mosaic modified virus-like particle (VLP) of cucumber mosaic virus (CMV). In a first aspect, the present invention provides modified virus-like particle (VLP) of cucumber mosaic virus (CMV) comprising (a) at least one fusion protein, wherein said at least one fusion protein comprises, or preferably consists of, a) a chimeric CMV polypeptide, wherein said chimeric CMV polypeptide comprises, or preferably consists of (i) a CMV polypeptide, wherein said CMV polypeptide comprises, or preferably consists of, a coat protein of CMV, wherein preferably said coat protein of CMV comprises, or preferably consists of, SEQ ID NO:62; or an amino acid sequence having a sequence identity of at least 75%, preferably of at least 80%, more preferably of at least 85%, again further preferably of at least 90%, again more preferably of at least 95%, still further preferably of at least 98% and still again further more preferably of at least 99% with SEQ ID NO:62; and (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and (b) at least one CMV protein, wherein said CMV protein comprises, or preferably consists of, a coat protein of CMV, wherein preferably said coat protein of CMV comprises, or preferably consists of, SEQ ID NO:62; or an amino acid sequence having a sequence identity of at least 75%, preferably of at least 80%, more preferably of at least 85%, again further preferably of at least 90%, again more preferably of at least 95%, still further preferably of at least 98% and still again further more preferably of at least 99% with SEQ ID NO:62, and wherein said CMV protein is optionally modified by a T helper cell epitope, wherein said T helper cell epitope replaces a N-terminal region of said CMV protein, and wherein preferably said N-terminal region of said CMV protein corresponds to amino acids 2-12 of SEQ ID NO:62; wherein preferably said CMV protein comprises, preferably consists of SEQ ID NO:5. In a very preferred embodiment, said chimeric CMV polypeptide further comprises a T helper cell epitope, wherein said T helper cell epitope replaces a N-terminal region of said CMV polypeptide, and wherein preferably said N-terminal region of said CMV polypeptide corresponds to amino acids 2-12 of SEQ ID NO:62. In a further very preferred embodiment, said chimeric CMV polypeptide further comprises a first amino acid linker, preferably a first amino acid linker and a second amino acid linker, wherein said first amino acid linker is positioned at the N- or at the C-terminus of said antigenic polypeptide, and wherein said first amino acid linker is selected from the group consisting of: (a.) a polyglycine linker (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker comprising at least one Gly, at least one Ser, and at least one amino acid selected from Thr, Ala, Lys, Asp and Glu.

For all aspects of the present invention, said insertion of said antigenic polypeptide between amino acid residues of said CMV polypeptide corresponding to amino acid residues of position 84 and position 85 of SEQ ID NO:62 corresponds to an insertion between said serine (S) residue being position 84 of SEQ ID NO:62 and said tyrosine (Y) residue being position 85 of SEQ ID NO:62.

The herein described and disclosed embodiments, preferred embodiments and very preferred embodiments should apply to all aspects and other embodiments, preferred embodiments and very preferred embodiments irrespective of whether is specifically again referred to or its repetition is avoided for the sake of conciseness.

In a preferred embodiment, said CMV polypeptide comprises, preferably consists of, an amino acid sequence of a coat protein of CMV or a mutated amino acid sequence, wherein the amino acid sequence to be mutated is an amino acid sequence of a coat protein of CMV, and wherein said mutated amino acid sequence and said coat protein of CMV show a sequence identity of at least 90%, preferably of at least 95%, further preferably of at least 98% and again more preferably of at least 99%; wherein preferably said mutated amino acid sequence and said amino acid sequence to be mutated differ in least one and in at most 11, 10, 9, 8, 7, 6, 5, 4, 3, or 2 amino acid residues, and wherein further preferably these differences are selected from (i) insertion, (ii) deletion, (iii) amino acid exchange, and (iv) any combination of (i) to (iii).

In a preferred embodiment, said CMV polypeptide comprises a coat protein of CMV or an amino acid sequence having a sequence identity of at least 75% with SEQ ID NO:62. In another preferred embodiment, said CMV polypeptide comprises a coat protein of CMV or an amino acid sequence having a sequence identity of at least 80% with SEQ ID NO:62. In another preferred embodiment, said CMV polypeptide comprises a coat protein of CMV or an amino acid sequence having a sequence identity of at least 85% with SEQ ID NO:62. In another preferred embodiment, said CMV polypeptide comprises a coat protein of CMV or an amino acid sequence having a sequence identity of at least 90% with SEQ ID NO:62. In another preferred embodiment, said CMV polypeptide comprises a coat protein of CMV or an amino acid sequence having a sequence identity of at least 95% with SEQ ID NO:62. In another preferred embodiment, said CMV polypeptide comprises a coat protein of CMV or an amino acid sequence having a sequence identity of at least 98% with SEQ ID NO:62. In another preferred embodiment, said CMV polypeptide comprises a coat protein of CMV or an amino acid sequence having a sequence identity of at least 99% with SEQ ID NO:62. In a preferred embodiment, said CMV polypeptide consists of a coat protein of CMV or an amino acid sequence having a sequence identity of at least 75% with SEQ ID NO:62. In another preferred embodiment, said CMV polypeptide consists of a coat protein of CMV or an amino acid sequence having a sequence identity of at least 80% with SEQ ID NO:62. In another preferred embodiment, said CMV polypeptide consists of a coat protein of CMV or an amino acid sequence having a sequence identity of at least 85% with SEQ ID NO:62. In another preferred embodiment, said CMV polypeptide consists of a coat protein of CMV or an amino acid sequence having a sequence identity of at least 90% with SEQ ID NO:62. In another preferred embodiment, said CMV polypeptide consists of a coat protein of CMV or an amino acid sequence having a sequence identity of at least 95% with SEQ ID NO:62. In another preferred embodiment, said CMV polypeptide consists of a coat protein of CMV or an amino acid sequence having a sequence identity of at least 98% with SEQ ID NO:62. In another preferred embodiment, said CMV polypeptide consists of a coat protein of CMV or an amino acid sequence having a sequence identity of at least 99% with SEQ ID NO:62. In a preferred embodiment, said CMV polypeptide is a coat protein of CMV or an amino acid sequence having a sequence identity of at least 75%, preferably 85% with SEQ ID NO:62. In a preferred embodiment, said CMV polypeptide is a coat protein of CMV or an amino acid sequence having a sequence identity of at least 90%, preferably 95% with SEQ ID NO:62. In a preferred embodiment, said CMV polypeptide is a coat protein of CMV with SEQ ID NO:62. In a preferred embodiment, said coat protein of CMV comprises SEQ ID NO:62. In a preferred embodiment, said coat protein of CMV consists of SEQ ID NO:62. In a preferred embodiment, said CMV polypeptide comprises a coat protein of CMV. In a preferred embodiment, said CMV polypeptide consists of a coat protein of CMV. In a preferred embodiment, said CMV polypeptide comprises a coat protein of CMV, wherein said coat protein of CMV comprises SEQ ID NO:62. In a preferred embodiment, said CMV polypeptide comprises a coat protein of CMV, wherein said coat protein of CMV consists of SEQ ID NO:62. In a preferred embodiment, said CMV polypeptide consists of a coat protein of CMV, wherein said coat protein of CMV consists of SEQ ID NO:62.

In a preferred embodiment, said CMV polypeptide comprises SEQ ID NO:63 or an amino acid sequence region, wherein said amino acid sequence region has a sequence identity of at least 75% with SEQ ID NO:63. In a preferred embodiment, said CMV polypeptide comprises SEQ ID NO:63 or an amino acid sequence region, wherein said amino acid sequence region has a sequence identity of at least 80% with SEQ ID NO:63. In a preferred embodiment, said CMV polypeptide comprises SEQ ID NO:63 or an amino acid sequence region, wherein said amino acid sequence region has a sequence identity of at least 85% with SEQ ID NO:63. In a preferred embodiment, said CMV polypeptide comprises SEQ ID NO:63 or an amino acid sequence region, wherein said amino acid sequence region has a sequence identity of at least 90% with SEQ ID NO:63. In a preferred embodiment, said CMV polypeptide comprises SEQ ID NO:63 or an amino acid sequence region, wherein said amino acid sequence region has a sequence identity of at least 95% with SEQ ID NO:63. In a preferred embodiment, said CMV polypeptide comprises SEQ ID NO:63 or an amino acid sequence region, wherein said amino acid sequence region has a sequence identity of at least 98% with SEQ ID NO:63. In a preferred embodiment, said CMV polypeptide comprises SEQ ID NO:63 or an amino acid sequence region, wherein said amino acid sequence region has a sequence identity of at least 99% with SEQ ID NO:63.

In a preferred embodiment, said CMV polypeptide comprises, or preferably consists of, (i) an amino acid sequence of a coat protein of CMV, wherein said amino acid sequence comprises, or preferably consists of, SEQ ID NO:62; or (ii) an amino acid sequence having a sequence identity of at least 90% of SEQ ID NO:62; and wherein said amino sequence as defined in (i) or (ii) comprises SEQ ID NO:63 or an amino acid sequence region, wherein said amino acid sequence region has a sequence identity of at least 90% with SEQ ID NO:63. In a preferred embodiment, said CMV polypeptide comprises, or preferably consists of, (i) an amino acid sequence of a coat protein of CMV, wherein said amino acid sequence comprises, or preferably consists of, SEQ ID NO:62; or (ii) an amino acid sequence having a sequence identity of at least 95% of SEQ ID NO:62; and wherein said amino sequence as defined in (i) or (ii) comprises SEQ ID NO:63 or an amino acid sequence region, wherein said amino acid sequence region has a sequence identity of at least 95% with SEQ ID NO:63. In a preferred embodiment, said CMV polypeptide comprises, or preferably consists of, (i) an amino acid sequence of a coat protein of CMV, wherein said amino acid sequence comprises, or preferably consists of, SEQ ID NO:62; or (ii) an amino acid sequence having a sequence identity of at least 90% of SEQ ID NO:62; and wherein said amino sequence as defined in (i) or (ii) comprises SEQ ID NO:63.

In a preferred embodiment, the number of amino acids of said N-terminal region replaced is equal to or lower than the number of amino acids of which said T helper cell epitope consists. In a preferred embodiment, said replaced N-terminal region of said CMV polypeptide consists of 5 to 15 consecutive amino acids. In a preferred embodiment, said replaced N-terminal region of said CMV polypeptide consists of 9 to 14 consecutive amino acids. In a preferred embodiment, said replaced N-terminal region of said CMV polypeptide consists of 11 to 13 consecutive amino acids. In a preferred embodiment, said N-terminal region of said CMV polypeptide corresponds to amino acids 2-12 of SEQ ID NO:62. In a preferred embodiment, said T helper cell epitope is a universal T helper cell epitope. In a preferred embodiment, said T helper cell epitope consists of at most 20 amino acids.

In a preferred embodiment of the present invention, the Th cell epitope is selected from HA 307-319 (SEQ ID NO:67), HBVnc 50-69 (SEQ ID NO:68), TT 830-843 (SEQ ID NO:64), CS 378-398 (SEQ ID NO:69), MT 17-31 (SEQ ID NO:70), TT 947-967 (SEQ ID NO:71) and PADRE (SEQ ID NO:65). In a very preferred embodiment, said Th cell epitope is a Th cell epitope derived from tetanus toxin or is a PADRE sequence. In a preferred embodiment, said T helper cell epitope is derived from a human vaccine. In a very preferred embodiment, said Th cell epitope is a Th cell epitope derived from tetanus toxin. In a preferred embodiment, said Th cell epitope is a PADRE sequence. In a very preferred embodiment, said Th cell epitope comprises the amino acid sequence of SEQ ID NO:64 or SEQ ID NO:65. In a very preferred embodiment, said Th cell epitope consists of the amino acid sequence of SEQ ID NO:64 or SEQ ID NO:65. In a very preferred embodiment, said Th cell epitope comprises the amino acid sequence of SEQ ID NO:64. In a preferred embodiment, said Th cell epitope consists of the amino acid sequence of SEQ ID NO:64. In a very preferred embodiment, said Th cell epitope comprises the amino acid sequence of SEQ ID NO:65. In a very preferred embodiment, said Th cell epitope consists of the amino acid sequence of SEQ ID NO:65.

In a preferred embodiment, said CMV polypeptide comprises, or preferably consists of, an amino acid sequence of a coat protein of CMV, wherein said amino acid sequence comprises, or preferably consists of, SEQ ID NO:62 or an amino acid sequence having a sequence identity of at least 95% of SEQ ID NO:62; and wherein said amino sequence comprises SEQ ID NO:63, and wherein said T helper cell epitope replaces the N-terminal region of said CMV polypeptide, and wherein said replaced N-terminal region of said CMV polypeptide consists of 11 to 13 consecutive amino acids, preferably of 11 consecutive amino acids, and wherein further preferably said N-terminal region of said CMV polypeptide corresponds to amino acids 2-12 of SEQ ID NO:62.

In a very preferred embodiment, said chimeric CMV polypeptide comprises the amino acid sequence of SEQ ID NO:5. In a very preferred embodiment, said chimeric CMV polypeptide comprises the amino acid sequence of SEQ ID NO:66.

In a very preferred embodiment, said chimeric CMV polypeptide comprises the amino acid sequence of SEQ ID NO:5 or SEQ ID NO:66, wherein said antigenic polypeptide is inserted into said chimeric CMV polypeptide of SEQ ID NO:5 between amino acid residues of position 88 and position 89 of SEQ ID NO:5 or wherein said antigenic polypeptide is inserted into said chimeric CMV polypeptide of SEQ ID NO:66 between amino acid residues of position 86 and position 87 of SEQ ID NO:66.

In a very preferred embodiment, said chimeric CMV polypeptide comprises the amino acid sequence of SEQ ID NO:66, wherein said antigenic polypeptide is inserted into said chimeric CMV polypeptide of SEQ ID NO:66 between amino acid residues of position 86 and position 87 of SEQ ID NO:66.

In a very preferred embodiment, said chimeric CMV polypeptide comprises the amino acid sequence of SEQ ID NO:5, wherein said antigenic polypeptide is inserted into said chimeric CMV polypeptide of SEQ ID NO:5 between amino acid residues of position 88 and position 89 of SEQ ID NO:5.

In a preferred embodiment, said chimeric CMV polypeptide further comprises a first amino acid linker, wherein said first amino acid linker is positioned at the N- or at the C-terminus of said antigenic polypeptide, and wherein said first amino acid linker is selected from the group consisting of: (a.) a polyglycine linker (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker comprising at least one Gly, at least one Ser, and at least one amino acid selected from Thr, Ala, Lys, Asp and Glu. In a preferred embodiment, said chimeric CMV polypeptide further comprises a second amino acid linker, wherein said second amino acid linker is positioned at the N- or at the C-terminus of said antigenic polypeptide, and wherein said second amino acid linker is selected from the group consisting of: (a.) a polyglycine linker (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker comprising at least one Gly, at least one Ser, and at least one amino acid selected from Thr, Ala, Lys, Asp and Glu. In a preferred embodiment, said chimeric CMV polypeptide further comprises a first amino acid linker and a second amino acid linker, wherein said first amino acid linker is positioned at the N-terminus of said antigenic polypeptide, and said second amino acid linker is positioned at the C-terminus of said antigenic polypeptide, and wherein said first and said second amino acid linker is independently selected from the group consisting of (a.) a polyglycine linker (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker comprising at least one Gly, at least one Ser, and at least one amino acid selected from Thr, Ala, Lys, Asp and Glu.

In a preferred embodiment, said first amino acid linker has a length of at most 30 amino acids. In a preferred embodiment, said first amino acid linker has a length of at most 20 amino acids. In a preferred embodiment, said first amino acid linker has a length of at most 15 amino acids. In a preferred embodiment, said second amino acid linker has a length of at most 30 amino acids. In a preferred embodiment, said second amino acid linker has a length of at most 20 amino acids. In a preferred embodiment, said second amino acid linker has a length of at most 15 amino acids. In a preferred embodiment, said first amino acid linker is positioned at the N-terminus of said antigenic polypeptide. In a preferred embodiment, said first amino acid linker is positioned at the C-terminus of said antigenic polypeptide. In a preferred embodiment, said chimeric CMV polypeptide further comprises a second amino acid linker. In a preferred embodiment, said first amino acid linker is positioned at the N-terminus of said antigenic polypeptide. In a preferred embodiment, said second amino acid linker is positioned at the C-terminus of said antigenic polypeptide. In a preferred embodiment, said chimeric CMV polypeptide comprises a first amino acid linker and a second amino acid linker. In a preferred embodiment, said first amino acid linker is positioned at the N-terminus of said antigenic polypeptide, and said second amino acid linker is positioned at the C-terminus of said antigenic polypeptide. In a preferred embodiment, said first amino acid linker is selected from the group consisting of: (a.) a polyglycine linker (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker comprising at least one Gly, at least one Ser, and at least one amino acid selected from Thr, Ala, Lys, Asp and Glu, wherein preferably said an amino acid linker comprising at least one Gly, at least one Ser, and at least one amino acid selected from Thr, Ala, Lys, Asp and Glu is a GST-linker or a GSED-linker. In a preferred embodiment, said first amino acid linker is a polyglycine linker (Gly)ₙ of a length of n=2-10. In a preferred embodiment, said first amino acid linker is a glycine-serine linker (GS-linker) comprising at least one glycine and at least one serine. In a preferred embodiment, said first amino acid linker is a glycine-serine linker (GS-linker) comprising at least one glycine and at least one serine, and said second amino acid linker has a Gly-Ser at its N-terminus. In a further preferred embodiment, said GS linker has an amino acid sequence of (GS)ᵣ(GₛS)ₜ(GS)ᵤ with r=0 or 1, s=1-5, t=1-5 and u=0 or 1. In a further preferred embodiment, said first amino acid linker is a glycine-serine linker (GS-linker), said GS linker has an amino acid sequence of (GS)ᵣ(GₛS)ₜ(GS)ᵤ with r=0 or 1, s=3 or 4, t=1, 2 or 3, and u=0 or 1. In a further preferred embodiment, said GS-linker has a length of at most 30 amino acids. In a further preferred embodiment, said GS-linker has a length of at most 20 amino acids. In a further preferred embodiment, said first amino acid linker is a glycine-serine linker (GS-linker), and said GS linker has an amino acid sequence selected from SEQ ID NO:10, SEQ ID NO:11, SEQ ID NO:30, SEQ ID NO:31 and SEQ ID NO:47. In a further preferred embodiment, said first amino acid linker has an amino acid sequence selected from SEQ ID NO:10 and SEQ ID NO:30. In a preferred embodiment, said first amino acid linker is an amino acid linker comprising at least one Gly, at least one Ser, and at least one amino acid selected from Thr, Ala, Lys, Asp and Glu. In a preferred embodiment, said first amino acid linker is an amino acid linker comprising at least one Gly, at least one Ser, and at least Thr (GST-linker), In a further preferred embodiment, said first amino acid linker is an amino acid linker comprising at least one Gly, at least one Ser, at least one glutamic acid and at least aspartic acid (GSED-linker), In a preferred embodiment, said first amino acid linker is an amino acid linker comprising at least one Gly, at least one Ser, and at least Thr (GST-linker), and said second amino acid linker has a Gly-Ser at its N-terminus. In a further preferred embodiment, said first amino acid linker is an amino acid linker comprising at least one Gly, at least one Ser, at least one glutamic acid and at least aspartic acid (GSED-linker), and said second amino acid linker has a Gly-Ser at its N-terminus. In a preferred embodiment, said first amino acid linker is a (GS-linker) comprising at least one glycine and at least one serine, an amino acid linker comprising at least one Gly, at least one Ser, and at least Thr (GST-linker) or an amino acid linker comprising at least one Gly, at least one Ser, at least one glutamic acid and at least aspartic acid (GSED-linker) and said second amino acid linker has a Gly-Ser at its N-terminus. In a further preferred embodiment, said first amino acid linker is a GSED linker, wherein said GSED-linker comprises an amino acid sequence of (DED)ₓ(GₛS)ₜ(G)_{y}(DED)_{z}(GS)ᵤ with s=1-5, t=1-5, u=0 or 1, x=0 or 1, y=0-5 and z=0 or 1. In a further preferred embodiment, said first amino acid linker is a GSED linker, wherein said GSED-linker comprises an amino acid sequence of (DED)ₓ(GₛS)ₜ(G)_{y}(DED)_{z}(GS)ᵤ with s=3 or 4, t=1, 2 or 3, u=0 or 1, x=0, y=1-5, preferably y=3, and z=1. In a further preferred embodiment, said GSED-linker has a length of at most 30 amino acids. In a further preferred embodiment, said GSED-linker has a length of at most 20 amino acids. In a further preferred embodiment, said first amino acid linker is a GSED-linker, and said GSED linker comprises, preferably consists of amino acid sequence SEQ ID NO:126.

In a preferred embodiment, said second amino acid linker is selected from the group consisting of: (a.) a polyglycine linker (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker comprising at least one Gly, at least one Ser, and at least one amino acid selected from Thr, Ala, Lys, Asp and Glu. In a preferred embodiment, said second amino acid linker is selected from the group consisting of: (a.) a polyglycine linker (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker comprising at least one Gly, at least one Ser, and at least one amino acid selected from Thr, Ala, Lys, Asp and Glu, wherein preferably said an amino acid linker comprising at least one Gly, at least one Ser, and at least one amino acid selected from Thr, Ala, Lys, Asp and Glu is a GST-linker or a GSED-linker. In a preferred embodiment, said second amino acid linker is a polyglycine linker (Gly)ₙ of a length of n=2-10. In a preferred embodiment, said second amino acid linker is a glycine-serine linker (GS-linker) consisting of at least one glycine and at least one serine. In a preferred embodiment, said second amino acid linker is a glycine-serine linker (GS-linker) comprising at least one glycine and at least one serine, and said second amino acid linker has a Gly-Ser at its N-terminus. In a further preferred embodiment, said second amino acid linker is a glycine-serine linker (GS-linker), said GS linker has an amino acid sequence of (GS)ᵣ(GₛS)ₜ(GS)ᵤ with r=0 or 1, s=3 or 4, t=1, 2 or 3, u=0 or 1. In a further preferred embodiment, said GS-linker has a length of at most 30 amino acids. In a further preferred embodiment, said GS-linker has a length of at most 20 amino acids. In a further preferred embodiment, said second amino acid linker is a glycine-serine linker (GS-linker), and said GS linker has an amino acid sequence selected from SEQ ID NO:10, SEQ ID NO:11, SEQ ID NO:30, SEQ ID NO:31 and SEQ ID NO:47. In a further preferred embodiment, said second amino acid linker has an amino acid sequence selected from SEQ ID NO:11, SEQ ID NO:31 and SEQ ID NO:47. In a further preferred embodiment, said GS linker has an amino acid sequence of (GS)ᵣ(GₛS)ₜ(GS)ᵤ with r=0 or 1, s=1-5, t=1-5 and u=0 or 1. In a preferred embodiment, said second amino acid linker is an amino acid linker comprising at least one Gly, at least one Ser, and at least one amino acid selected from Thr, Ala, Lys, Asp and Glu.

In a preferred embodiment, said second amino acid linker is an amino acid linker comprising at least one Gly, at least one Ser, and at least Thr (GST-linker), In a further preferred embodiment, said second amino acid linker is an amino acid linker comprising at least one Gly, at least one Ser, at least one glutamic acid and at least aspartic acid (GSED-linker), In a preferred embodiment, said second amino acid linker is an amino acid linker comprising at least one Gly, at least one Ser, and at least Thr (GST-linker), and said second amino acid linker has a Gly-Ser at its N-terminus. In a further preferred embodiment, said second amino acid linker is an amino acid linker comprising at least one Gly, at least one Ser, at least one glutamic acid and at least aspartic acid (GSED-linker), and said second amino acid linker has a Gly-Ser at its N-terminus. In a preferred embodiment, said second amino acid linker is a (GS-linker) comprising at least one glycine and at least one serine, an amino acid linker comprising at least one Gly, at least one Ser, and at least Thr (GST-linker) or an amino acid linker comprising at least one Gly, at least one Ser, at least one glutamic acid and at least aspartic acid (GSED-linker) and said second amino acid linker has a Gly-Ser at its N-terminus. In a further preferred embodiment, said second amino acid linker is a GSED linker, wherein said GSED-linker comprises an amino acid sequence of (DED)ₓ(GₛS)ₜ(G)_{y}(DED)_{z}(GS)ᵤ with s=1-5, t=1-5, u=0 or 1, x=0 or 1, y=0-5 and z=0 or 1. In a further preferred embodiment, said second amino acid linker is a GSED linker, wherein said GSED-linker consists of an amino acid sequence of (DED)ₓ(GₛS)ₜ(G)_{y}(DED)_{z}(GS)ᵤ with s=1-5, t=1-5, u=0 or 1, x=0 or 1, y=0-5 and z=0 or 1. In a further preferred embodiment, said second amino acid linker is a GSED linker, wherein said GSED-linker comprises an amino acid sequence of (DED)ₓ(GₛS)ₜ(G)_{y}(DED)_{z}(GS)ᵤ with s=3 or 4, t=1, 2 or 3, u=0 or 1, x=1, y=0-5, preferably y=0, and z=0. In a further preferred embodiment, said second amino acid linker is a GSED linker, wherein said GSED-linker consists of an amino acid sequence of (DED)ₓ(GₛS)ₜ(G)_{y}(DED)_{z}(GS)ᵤ with s=3 or 4, t=1, 2 or 3, u=0 or 1, x=1, y=0-5, preferably y=0, and z=0. In a further preferred embodiment, said second amino acid linker is a GSED linker, wherein said GSED-linker comprises, preferably consists of, an amino acid sequence of (TS)(DED)ₓ(GₛS)ₜ(G)_{y}(DED)_{z}(GS)ᵤ with s=1-5, t=1-5, u=0 or 1, x=0 or 1, y=0-5 and z=0 or 1. In a further preferred embodiment, said second amino acid linker is a GSED linker, wherein said GSED-linker comprises, preferably consists of, an amino acid sequence of (TS)(DED)ₓ(GₛS)ₜ(G)_{y}(DED)_{z}(GS)ᵤ with s=3 or 4, t=1, 2 or 3, u=0 or 1, x=1, y=0-5, preferably y=0, and z=0. In a further preferred embodiment, said GSED-linker has a length of at most 30 amino acids. In a further preferred embodiment, said GSED-linker has a length of at most 20 amino acids. In a further preferred embodiment, said second amino acid linker is a GSED-linker, and said GSED linker comprises, preferably consists of amino acid sequence SEQ ID NO:127.

In a preferred embodiment, said first and said second amino acid linker is independently selected from the group consisting of: (a.) a polyglycine linker (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker comprising at least one Gly, at least one Ser, and at least one amino acid selected from Thr, Ala, Lys, Asp and Glu. In a preferred embodiment, said first and said second amino acid linker is independently a polyglycine linker (Gly)ₙ of a length of n=2-10. In a preferred embodiment, said first and said second amino acid linker is independently a glycine-serine linker (GS-linker) comprising at least one glycine and at least one serine. In a preferred embodiment, said first and said second amino acid linker is independently a glycine-serine linker (GS-linker) comprising at least one glycine and at least one serine, and wherein said second amino acid linker has a Gly-Ser at its N-terminus. In a further preferred embodiment, said GS linker has an amino acid sequence of (GS)ᵣ(GₛS)ₜ(GS)ᵤ with r=0 or 1, s=1-5, t=1-5 and u=0 or 1. In a further preferred embodiment, said first and said second amino acid linker is independently a glycine-serine linker (GS-linker), said GS linker has an amino acid sequence of (GS)ᵣ(GₛS)ₜ(GS)ᵤ with r=0 or 1, s=3 or 4, t=1, 2 or 3, u=0 or 1. In a further preferred embodiment, said first and said second amino acid linker is independently a glycine-serine linker (GS-linker), and said GS linker has an amino acid sequence selected from SEQ ID NO:10, SEQ ID NO:11, SEQ ID NO:30, SEQ ID NO:31 and SEQ ID NO:47. In a preferred embodiment, said first and said second amino acid linker is independently an amino acid linker comprising at least one Gly, at least one Ser, and at least one amino acid selected from Thr, Ala, Lys, Asp and Glu.

In a preferred embodiment, said first and said second amino acid linker is independently a GSED-linker comprising at least one Gly, at least one Ser, at least one glutamic acid and at least aspartic acid (GSED-linker), and said second amino acid linker has a Gly-Ser at its N-terminus. In a further preferred embodiment, said first and said second amino acid linker is independently a GSED-linker, wherein said GSED linker has independently an amino acid sequence of (DED)ₓ(GₛS)ₜ(G)_{y}(DED)_{z}(GS)ᵤ with s=1-5, t=1-5, u=0 or 1, x=0 or 1, y=0-5 and z=0 or 1; or of (TS)(DED)ₓ(GₛS)ₜ(G)_{y}(DED)_{z}(GS)ᵤ with s=1-5, t=1-5, u=0 or 1, x=0 or 1, y=0-5 and z=0 or 1.

In a further preferred embodiment, said first and said second amino acid linker is independently a GSED-linker, wherein said GSED linker has independently an amino acid sequence of (DED)ₓ(GₛS)ₜ(G)_{y}(DED)_{z}(GS)ᵤ with s=3 or 4, t=1, 2 or 3, u=0 or 1, x=1, y=0-5, preferably y=0, and z=0, or of s=3 or 4, t=1, 2 or 3, u=0 or 1, x=0, y=1-5, preferably y=3, and z=1; or of (TS)(DED)ₓ(GₛS)ₜ(G)_{y}(DED)_{z}(GS)ᵤ with s=3 or 4, t=1, 2 or 3, u=0 or 1, x=1, y=0-5, preferably y=0, and z=0.

In a further preferred embodiment, said first and said second amino acid linker is independently a GSED-linker, and said GS linker has an amino acid sequence selected from SEQ ID NO:126 and SEQ ID NO:127.

In a very preferred embodiment, said chimeric CMV polypeptide comprises the amino acid sequence of SEQ ID NO:5 and said antigenic polypeptide is inserted into said CMV polypeptide between amino acid residues 88 (Ser) and amino acid residue 89 (Thr) of said CMV polypeptide of SEQ ID NO:5.

In a very preferred embodiment, said chimeric CMV polypeptide comprises the amino acid sequence of SEQ ID NO:5 and said antigenic polypeptide is inserted into said CMV polypeptide between amino acid residues 88 (Ser) and amino acid residue 89 (Thr) of said CMV polypeptide of SEQ ID NO:5, and said chimeric CMV polypeptide further comprises a first and a second amino acid linker, wherein said first and said second amino acid linker is independently a glycine-serine linker (GS-linker) comprising at least one glycine and at least one serine, and wherein said second amino acid linker has a Gly-Ser sequence at its N-terminus.

In an aspect and preferred embodiment, the invention provides for mosaic virus-like particles. Thus, in a preferred embodiment, said modified VLP of CMV further comprises at least one CMV protein, wherein said CMV protein comprises, or preferably consists of, a coat protein of CMV, wherein preferably said coat protein of CMV comprises, or preferably consists of, SEQ ID NO:62; or an amino acid sequence having a sequence identity of at least 75%, preferably of at least 80%, more preferably of at least 85%, again further preferably of at least 90%, again more preferably of at least 95%, still further preferably of at least 98% and still again further more preferably of at least 99% with SEQ ID NO:62. In a preferred embodiment, said modified VLP of CMV further comprises at least one CMV protein, wherein said CMV protein comprises a coat protein of CMV or an amino acid sequence having a sequence identity of at least 75%, preferably of at least 85% with SEQ ID NO:62, and wherein said CMV protein is optionally modified by a T helper cell epitope, and wherein preferably said coat protein of CMV comprises SEQ ID NO:62.

In a preferred embodiment, said CMV protein comprises a coat protein of CMV or an amino acid sequence having a sequence identity of at least 75% with SEQ ID NO:62. In another preferred embodiment, said CMV protein comprises a coat protein of CMV or an amino acid sequence having a sequence identity of at least 80% with SEQ ID NO:62. In another preferred embodiment, said CMV protein comprises a coat protein of CMV or an amino acid sequence having a sequence identity of at least 85% with SEQ ID NO:62. In another preferred embodiment, said CMV protein comprises a coat protein of CMV or an amino acid sequence having a sequence identity of at least 90% with SEQ ID NO:62. In another preferred embodiment, said CMV protein comprises a coat protein of CMV or an amino acid sequence having a sequence identity of at least 95% with SEQ ID NO:62. In another preferred embodiment, said CMV protein comprises a coat protein of CMV or an amino acid sequence having a sequence identity of at least 98% with SEQ ID NO:62. In another preferred embodiment, said CMV protein comprises a coat protein of CMV or an amino acid sequence having a sequence identity of at least 99% with SEQ ID NO:62. In a preferred embodiment, said CMV protein comprises a coat protein of CMV. In a preferred embodiment, said CMV protein consists of a coat protein of CMV. In a preferred embodiment, said CMV protein comprises a coat protein of CMV, wherein said coat protein of CMV comprises SEQ ID NO:62. In a preferred embodiment, said CMV protein comprises a coat protein of CMV, wherein said coat protein of CMV consists of SEQ ID NO:62. In a preferred embodiment, said CMV protein consists of a coat protein of CMV, wherein said coat protein of CMV consists of SEQ ID NO:62. In another preferred embodiment, said CMV protein is modified by a T helper cell epitope, wherein said T helper cell epitope replaces a N-terminal region of said CMV protein. In another preferred embodiment, said N-terminal region of said CMV protein corresponds to amino acids 2-12 of SEQ ID NO:62. In a very preferred embodiment, said CMV protein comprises SEQ ID NO:5. In a very preferred embodiment, said CMV protein consists of SEQ ID NO:5.

In a preferred embodiment, said antigenic polypeptide has a length of at least 3 amino acids. In a preferred embodiment, said antigenic polypeptide has a length of at least 3 amino acids and at most 225 amino acids. In a preferred embodiment, said antigenic polypeptide has a length of at least 3 amino acids and at most 200 amino acids. In a preferred embodiment, said antigenic polypeptide has a length of at least 40 amino acids. In a preferred embodiment, said antigenic polypeptide has a length of at least 40 amino acids and at most 225 amino acids, preferably at most 200 amino acids. In a preferred embodiment, said antigenic polypeptide has a length of at least 50 amino acids. In a preferred embodiment, said antigenic polypeptide has a length of at least 50 amino acids and at most 200 amino acids. In a preferred embodiment, said antigenic polypeptide has a length of at least 70 amino acids. In a preferred embodiment, said antigenic polypeptide has a length of at least 70 amino acids and at most 200 amino acids.

In a preferred embodiment, said antigenic polypeptide is a polypeptide derived from the group consisting of: (a) allergens; (b) viruses; (b) bacteria; (c) parasites; (d) tumors; (e) self-molecules; (h) hormones; (i) cytokines; (k) chemokines; (l) biologically active peptides.

In another preferred embodiment, said antigenic polypeptide is an allergen, a self antigen, a tumor antigen, or a polypeptide of a pathogen.

In a further preferred embodiment said antigenic polypeptide is an allergen, wherein preferably said allergen is derived from the group consisting of: (a) pollen extract; (b) dust extract; (c) dust mite extract; (d) fungal extract; (e) mammalian epidermal extract; (f) feather extract; (g) insect extract; (h) food extract; (i) hair extract; (j) saliva extract; and (k) serum extract. In a further preferred embodiment said antigenic polypeptide is an allergen, wherein said allergen is selected from the group consisting of: (a) trees; (b) grasses; (c) house dust; (d) house dust mite; (e) Aspergillus; (f) animal hair; (g) animal feather; (h) bee venom; (i) animal products; (j) plant products; (k) animal dander; (l) peanut allergens.

In a further preferred embodiment said antigenic polypeptide is a recombinant polypeptide derived from an allergen selected from the group consisting of: (a) bee venom phospholipase A2; (b) ragweed pollen Amb a 1; (c) birch pollen Bet v I; (d) white faced hornet venom 5 DoI m V; (e) house dust mite Der p 1; (f) house dust mite Der f 2; (g) house dust mite Der p 2; (h) dust mite Lep d; (i) fungus allergen Alt a 1; (j) fungus allergen Asp f 1; (k) fungus allergen Asp f 16; (l) peanut allergens (m) cat allergen Fel d1; (n) Canine allergens Can f1, Can f2 (o) peanut-derived allergens; or (p) Japanese cedar allergen Cry J2.

In a further preferred embodiment said antigenic polypeptide is a recombinant allergen, wherein said allergen is selected from the group consisting of: (a) bee venom phospholipase A2; (b) ragweed pollen Amb a 1; (c) birch pollen Bet v I; (d) white faced hornet venom 5 DoI m V; (e) house dust mite Der p 1; (f) house dust mite Der f 2; (g) house dust mite Der p 2; (h) dust mite Lep d; (i) fungus allergen Alt a 1; (j) fungus allergen Asp f 1; (k) fungus allergen Asp f 16; (l) peanut allergens (m) cat allergen Fel d1; (n) Canine allergens Can f1, Can f2 (o) peanut-derived allergens; or (p) Japanese cedar allergen Cry J2.

In a further very preferred embodiment, said antigenic polypeptide is a peanut allergen. Preferably, said antigenic polypeptide is a peanut allergen comprising an amino acid sequence selected from SEQ ID NO:27, SEQ ID NO:72 or SEQ ID NO:73. In a very preferred embodiment, said peanut allergen comprises, or preferably consists of, a protein with the amino sequence selected from SEQ ID NO:27, SEQ ID NO:72 or SEQ ID NO:73 or a protein with an amino acid sequence of at least 90%, preferably of at least 92%, further preferably of at least 95%, and again further preferably of at least 98% amino acid sequence identity with SEQ ID NO:27, SEQ ID NO:72 or SEQ ID NO:73. In a further very preferred embodiment, said peanut allergen comprises, or preferably consists of, a protein with the amino sequence selected from SEQ ID NO:27, SEQ ID NO:72 or SEQ ID NO:73, or a protein with an amino acid sequence of at least 90%, preferably of at least 92%, further preferably of at least 95%, and again further preferably of at least 98% amino acid sequence identity with SEQ ID NO:27, SEQ ID NO:72 or SEQ ID NO:73. In a further very preferred embodiment, said peanut allergen comprises a protein with the amino sequence selected from SEQ ID NO:27, SEQ ID NO:72 or SEQ ID NO:73. In a further very preferred embodiment, said peanut allergen consists of a protein with the amino sequence selected from SEQ ID NO:27, SEQ ID NO:72 or SEQ ID NO:73. In a further very preferred embodiment, said peanut allergen comprises the amino acid sequence of SEQ ID NO:27. In a further very preferred embodiment, said peanut allergen consists of the amino acid sequence of SEQ ID NO:27. In a further very preferred embodiment, said antigenic polypeptide comprises the amino acid sequence of SEQ ID NO:27. In a further very preferred embodiment, said antigenic polypeptide consists of the amino acid sequence of SEQ ID NO:27.

In a very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62; or an amino acid sequence having a sequence identity of at least 98% with SEQ ID NO:62, preferably wherein said CMV polypeptide comprises, or preferably consists of, SEQ ID NO:62; (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is a peanut allergen; and wherein said peanut allergen comprises an amino acid sequence selected from: (a) SEQ ID NO:27; (b) SEQ ID NO:72 (c) SEQ ID NO:73, and wherein preferably said a peanut allergen comprises, preferably consists of SEQ ID NO:27; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of the amino acid sequence of SEQ ID NO:64 or SEQ ID NO:65, again preferably of SEQ ID NO:64.

In a further very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is a peanut allergen; and wherein said peanut allergen comprises, preferably consists of SEQ ID NO:27; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:29. In a further very preferred embodiment and aspect hereto, the present invention provides said modified virus-like particle (VLP) of cucumber mosaic virus (CMV) for use in method of treating allergy, preferably peanut allergy.

In a further very preferred embodiment, the modified virus-like particle (VLP) of cucumber mosaic virus (CMV) comprises at least one fusion protein and further comprises at least one CMV protein, wherein said at least one fusion protein comprises, or preferably consists of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is a peanut allergen; and wherein said peanut allergen comprises, preferably consists of SEQ ID NO:27; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64; and wherein said CMV protein comprises, preferably consists of, a coat protein of CMV, preferably of SEQ ID NO:62, and wherein said CMV protein is optionally modified by a T helper cell epitope, and wherein said T helper cell epitope replaces a N-terminal region of said CMV protein, wherein said N-terminal region of said CMV protein corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:29 and said CMV protein consists of SEQ ID NO:5. In a further very preferred embodiment and aspect hereto, the present invention provides said modified virus-like particle (VLP) of cucumber mosaic virus (CMV) for use in method of treating allergy, preferably peanut allergy.

In a further very preferred embodiment, said antigenic polypeptide is an allergen derived from Japanese Cedar Cry J 2. Preferably, said antigenic polypeptide is derived from Japanese Cedar Cry J 2 of SEQ ID NO:74. Preferably, said antigenic polypeptide is derived from Japanese Cedar Cry J 2 and comprises the amino acid sequence of SEQ ID NO:74.

In a further very preferred embodiment, said antigenic polypeptide is an allergen derived from ragweed pollen Amb al. Preferably, said antigenic polypeptide is derived from ragweed pollen Amb a 1 of SEQ ID NO:75. Preferably, said antigenic polypeptide is derived from ragweed pollen Amb al and comprises the amino acid sequence of SEQ ID NO:75.

In a very preferred embodiment of the present invention, said antigenic polypeptide is an allergen derived from cat allergen Fel d1. The domestic cat (Fells domesticus) is an important source of indoor allergens (Lau, S., et al. (2000) Lancet 356, 1392-1397). The severity of symptoms range from relatively mild rhinitis and conjunctivitis to potentially life-threatening asthmatic exacerbation. Although patients are occasionally sensitized to several different molecules in cat dander and pelts, the major allergen is Fel d1. The importance of this allergen has been emphasised in numerous studies. In fact more than 80% of cat allergic patients exhibit IgE antibodies to this potent allergen (van Ree, R., et al. (1999) J. Allergy Clin Immunol 104, 1223-1230). Fel d1 is a 35-39 kDa acidic glycoprotein containing 10-20% N-linked carbohydrates and is found in the pelt, saliva and lachrymal glands of cats. It is formed by two non-covalently linked heterodimers. Each heterodimer consists of one 70 residue peptide (known as “chain 1”) and one 78, 85, 90 or 92 residue peptide (known as “chain 2”) which are encoded by separate genes (see Duffort, 0. A., et al. (1991) Mol Immunol 28, 301-309; Morgenstern, J. P., et al; (1991) Proc Natl Acad Sci USA 88, 9690-9694 and Griffith, I. J., et al. (1992) Gene 113, 263-268). Several recombinant constructs of Fel d1 have been described (Vailes L D, et al., J Allergy Clin Immunol (2002) 110:757-762; Grönlund H, et al., J Biol Chem (2003) 278:40144-40151; 2003; Schmitz N, et al., J Exp Med (2009) 206:1941-1955; WO2006/097530; WO2017/042241).

Thus, in a further very preferred embodiment, said antigenic polypeptide is a rFel d1. In a further very preferred embodiment, said antigenic polypeptide is a Fel d1 protein, wherein said Fel d1 protein is a fusion protein comprising chain 1 of Fel d1 and chain 2 of Fel d1, wherein said chain 2 of Fel d1 is fused via its C-terminus to the N-terminus of said chain 1 of Fel d1 either directly via one peptide bond or via a spacer, wherein said spacer consists of an amino acid sequence having 1-20 amino acid residues, wherein preferably said spacer consists of an amino acid sequence having 10-20 amino acid residues. Very preferably, said spacer consists of an amino acid sequence of 15 amino acid residues, and further preferably said spacer has the amino acid sequence of SEQ ID NO:30. In a further very preferred embodiment, said antigenic polypeptide is a Fel d1 protein, wherein said Fel d1 protein is a fusion protein comprising chain 1 of Fel d1 and chain 2 of Fel d1, wherein said chain 1 of Fel d1 is fused via its C-terminus to the N-terminus of said chain 2 of Fel d1 either directly via one peptide bond or via a spacer, wherein said spacer consists of an amino acid sequence having 1-20 amino acid residues, wherein preferably said spacer consists of an amino acid sequence having 10-20 amino acid residues. Preferably, said chain 1 of Fel d1 comprises a sequence of SEQ ID NO:76 or a homologue sequence thereof, wherein said homologue sequence has an identity to SEQ ID NO:76 of greater than 90%, or even more preferably greater than 95%. Further preferably, said chain 2 of Fel d 1 comprises a sequence of SEQ ID NO:77, SEQ ID NO:78 or SEQ ID NO:79, or a homologue sequence thereof, wherein said homologue sequence has an identity to SEQ ID NO:77, SEQ ID NO:78 or SEQ ID NO:79 of greater than 90%, and even more preferably greater than 95%.

In a very preferred embodiment said antigenic polypeptide is a Fel d1 protein comprising an amino acid sequence selected from: (a) SEQ ID NO:38; (b) SEQ ID NO:80; or (c) or SEQ ID NO:81. In another very preferred embodiment, said antigenic polypeptide comprises, preferably consists of, SEQ ID NO:38. In a very preferred embodiment, said antigenic polypeptide comprises, preferably consists of, SEQ ID NO:80. In another very preferred embodiment, said antigenic polypeptide comprises, preferably consists of, SEQ ID NO:81.

In a further very preferred embodiment, said Fel d1 protein comprises an amino acid sequence selected from: (a) SEQ ID NO:38; (b) SEQ ID NO:80 (c) SEQ ID NO:81.

In another very preferred embodiment, said Fel d1 protein comprises, preferably consists of, an amino acid sequence of SEQ ID NO:38. In another very preferred embodiment, said Fel d1 protein comprises, preferably consists of, an amino acid sequence of SEQ ID NO:80. In another very preferred embodiment, said Fel d1 protein comprises, preferably consists of, an amino acid sequence of SEQ ID NO:81.

In a very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62; or an amino acid sequence having a sequence identity of at least 98% with SEQ ID NO:62, preferably wherein said CMV polypeptide comprises, or preferably consists of, SEQ ID NO:62; (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is a Fel d1 protein; and wherein said Fel d1 protein comprises an amino acid sequence selected from: (a) SEQ ID NO:38; (b) SEQ ID NO:80 (c) SEQ ID NO:81, and wherein preferably said Fel d1 protein comprises, preferably consists of SEQ ID NO:38; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of the amino acid sequence of SEQ ID NO:64 or SEQ ID NO:65, again preferably of SEQ ID NO:64.

In a further very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is a Fel d1 protein; and wherein said Fel d1 protein comprises, preferably consists of SEQ ID NO:38; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:39. In a further very preferred embodiment and aspect hereto, the present invention provides said modified virus-like particle (VLP) of cucumber mosaic virus (CMV) for use in method of treating allergy, preferably cat allergy.

In a further very preferred embodiment, the modified virus-like particle (VLP) of cucumber mosaic virus (CMV) comprises at least one fusion protein and further comprises at least one CMV protein, wherein said at least one fusion protein comprises, or preferably consists of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is a Fel d1 protein; and wherein said Fel d1 protein comprises, preferably consists of SEQ ID NO:38; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64; and wherein said CMV protein comprises, preferably consists of, a coat protein of CMV, preferably of SEQ ID NO:62, and wherein said CMV protein is optionally modified by a T helper cell epitope, and wherein said T helper cell epitope replaces a N-terminal region of said CMV protein, wherein said N-terminal region of said CMV protein corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:39 and said CMV protein consists of SEQ ID NO:5. In a further very preferred embodiment and aspect hereto, the present invention provides said modified virus-like particle (VLP) of cucumber mosaic virus (CMV) for use in method of treating allergy, preferably cat allergy.

In a further preferred embodiment said antigenic polypeptide is a tumor antigen, wherein preferably said tumor antigen is selected from the group consisting of: (a) a polypeptide of breast cancer cells; (b) a polypeptide of kidney cancer cells; (c) a polypeptide of prostate cancer cells; (d) a polypeptide of skin cancer cells; (e) a polypeptide of brain cancer cells; and (f) a polypeptide of leukemia cells.

In a further preferred embodiment said antigenic polypeptide is a tumor antigen selected from the group consisting of: (a) Her2; (b) ganglioside GD2; (c) EGF-R; (d) carcino embryonic antigen (CEA); (e) CD52; (f) CD21; (g) human melanoma gp100; (h) human melanoma melanA/MART-1; (i) Human melanoma melanA/MART-1 analogue; (j) tyrosinase; (k) NA17-A nt; (l) MAGE3; (m) p53 protein; and (n) antigenic fragments of any of the tumor antigens of (a) to (m).

In a further preferred embodiment said antigenic polypeptide is a polypeptide selected from the group consisting of: (a) IgE, (b) IL-6 (c) receptor activator of nuclear factor kB ligand (RANKL); (d) vascular endothelial growth factor (VEGF); (e) vascular endothelial growth factor receptor (VEGF-R); hepatocyte growth factor (HGF) (f) interleukin-1α; (g) interleukin-1 β; (h) interleukin-5; (i) interleukin-8; (j) interleukin-13; (k) interleukin-15; (l) interleukin-17 (IL-17); (m) IL-23; (n) Ghrelin; (o) angiotensin; (p) chemokine (C—C motif) (CCL21); (q) chemokine (C—X motif) (CXCL 12); (r) stromal cell derived factor 1 (SDF-I); (s) macrophage colony stimulating factor (M-CSF); (t) monocyte chemotactic protein 1 (MCP-I); (u) endoglin; (v) resistin; (w) gonadotropin releasing hormone (GnRH); (x) growth hormone releasing (GHRH); (y) lutenizing hormone releasing hormone (LHRH); (z) thyreotropin releasing hormone (TRH); (aa) macrophage migration inhibitory factor (MIF); (bb) glucose-dependent insulinotropic peptide (GIP); (cc) eotaxin; (dd) bradykinin; (ee) Des-Arg bradykinin; (ff) B-lymphocyte chemoattractant (BLC); (gg) macrophage colony stimulating factor M-CSF; (hh) tumor necrosis factor α (TNFα); (ii) amyloid beta peptide (Aβ1-42); (jj) amyloid beta peptide (Aβ3-6); (kk) human IgE; (ii) CCR5 extracellular domain; (mm) CXCR4 extracellular domain; (nn) Gastrin; (oo) CETP; (pp) C5a; (qq) epidermal growth factor receptor (EGF-R); (rr) CGRP; (ss) α-synuclein; (tt) calcitonin gene-related peptide (CGRP) (uu) Amylin; (vv) myostatin; (ww) interleukin-4; (xx) thymic stromal lymphopoietin; (yy) interleukin-33; (zz) interleukin-25; (aaa) interleukin-13 or (bbb) a fragment of any one of the polypeptides (a) to (aaa); and (ccc) an antigenic mutant or fragment of any one of the polypeptides (a) to (aaa).

In a further preferred embodiment said antigenic polypeptide is a self antigen, wherein said self antigen is a polypeptide selected from the group consisting of: (a) IgE, (b) IL-6 (c) receptor activator of nuclear factor kB ligand (RANKL); (d) vascular endothelial growth factor (VEGF); (e) vascular endothelial growth factor receptor (VEGF-R); hepatocyte growth factor (HGF) (f) interleukin-1 α; (g) interleukin-1 β; (h) interleukin-5; (i) interleukin-8; (j) inter leukin-13; (k) interleukin-15; (l) interleukin-17 (IL-17); (m) IL-23; (n) Ghrelin; (o) angiotensin; (p) chemokine (C—C motif) (CCL21); (q) chemokine (C—X motif) (CXCL 12); (r) stromal cell derived factor 1 (SDF-I); (s) macrophage colony stimulating factor (M-CSF); (t) monocyte chemotactic protein 1 (MCP-I); (u) endoglin; (v) resistin; (w) gonadotropin releasing hormon (GnRH); (x) growth hormon releasing (GHRH); (y) lutenizing hormon releasing hormon (LHRH); (z) thyreotropin releasing hormon (TRH); (aa) macrophage migration inhibitory factor (MIF); (bb) glucose-dependent insulinotropic peptide (GIP); (cc) eotaxin; (dd) bradykinin; (ee) Des-Arg bradykinin; (ff) B-lymphocyte chemoattractant (BLC); (gg) macrophage colony stimulating factor M-CSF; (hh) tumor necrosis factor α (TNFα); (ii) amyloid beta peptide (Aβ1-42); (jj) amyloid beta peptide (Aβ3-6); (kk) human IgE; (ii) CCR5 extracellular domain; (mm) CXCR4 extracellular domain; (nn) Gastrin; (oo) CETP; (pp) C5a; (qq) epidermal growth factor receptor (EGF-R); (rr) CGRP; (ss) α-synuclein; (tt) calcitonin gene-related peptide (CGRP) (uu) Amylin; (vv) myostatin; (ww) interleukin-4; (xx) thymic stromal lymphopoietin; (yy) interleukin-33; (zz) interleukin-25; (aaa) interleukin-13 or (bbb) a fragment of any one of the polypeptides (a) to (aaa); and (ccc) an antigenic mutant or fragment of any one of the polypeptides (a) to (aaa).

In a preferred embodiment, said antigenic polypeptide is interleukin 17 (IL-17), preferably human IL-17. Interleukin 17 is a T cell-derived cytokine that induces the release of pro-inflammatory mediators in a wide range of cell types. Aberrant Th17 responses and overexpression of IL-17 have been implicated in a number of autoimmune disorders including rheumatoid arthritis and multiple sclerosis. Molecules blocking IL-17 such as IL-17-specific monoclonal antibodies have proved to be effective in ameliorating disease in animal models. Moreover, active immunization targeting IL-17 has recently been suggested using virus-like particles conjugated with recombinant IL-17 (Röhn T A, et al., Eur J Immunol (2006) 36: 1-11). Immunization with IL-17-VLP induced high levels of anti-IL-17 antibodies thereby overcoming natural tolerance, even in the absence of added adjuvant. Mice immunized with IL-17-VLP had lower incidence of disease, slower progression to disease and reduced scores of disease severity in both collagen-induced arthritis and experimental autoimmune encephalomyelitis. Thus, in a preferred embodiment, said antigenic polypeptide comprises, or preferably consists of SEQ ID NO:82. Furthermore, the inventive modified CMV VLPs are used in a method of treating an inflammatory disease, preferably a chronic inflammatory disease in an animal or human. Preferably, said inflammatory disease is selected from RA, MS, Psoriasis, asthma, Crohns, Colitis, COPD, diabetes, neurodermatitis (allergic dermatitis), again preferably wherein said inflammatory disease MS, and wherein further preferably said antigenic polypeptide comprises, or preferably consists of SEQ ID NO:82.

In another preferred embodiment, said antigenic polypeptide is IL-5, preferably human IL-5. In again a further preferred embodiment, said antigenic polypeptide comprises, or preferably consists of SEQ ID NO:83. Furthermore, the inventive modified VLPs are used in a method of treating an inflammatory disease, preferably a chronic inflammatory disease in an animal or human. Preferably, said inflammatory disease is selected from RA, MS, Psoriasis, asthma, Crohns, Colitis, COPD, diabetes, neurodermatitis (allergic dermatitis), and wherein further preferably said antigenic polypeptide comprises, or preferably consists of SEQ ID NO:83.

In another preferred embodiment, said antigenic polypeptide is canine IL-5. In again a further preferred embodiment, said antigenic polypeptide comprises, or preferably consists of SEQ ID NO:84 or an amino acid sequence having a sequence identity of at least 90%, preferably of at least 95%, with SEQ ID NO:84. Preferably, said antigenic polypeptide comprises, or preferably consists of SEQ ID NO:84. In another preferred embodiment, said antigenic polypeptide consists of SEQ ID NO:84.

In another preferred embodiment, said antigenic polypeptide is feline IL-5. In a very preferred embodiment, said antigenic polypeptide comprises, or preferably consists of, SEQ ID NO:85, SEQ ID NO:125, SEQ ID:141 or an amino acid sequence having a sequence identity of at least 90%, preferably of at least 92%, further preferably of at least 95%, and again further preferably of at least 98% amino acid sequence identity with SEQ ID NO:85, SEQ ID NO:125, SEQ ID:141. In a further very preferred embodiment, said antigenic polypeptide comprises SEQ ID NO:85, SEQ ID NO:125 or SEQ ID:141. In a further very preferred embodiment, said antigenic polypeptide consists of SEQ ID NO:85, SEQ ID NO:125 or SEQ ID:141. In a further very preferred embodiment, said antigenic polypeptide comprises, or preferably consists of, SEQ ID NO:85 or an amino acid sequence having a sequence identity of at least 90%, preferably of at least 92%, further preferably of at least 95%, and again further preferably of at least 98% amino acid sequence identity with SEQ ID NO:85. In a further very preferred embodiment, said antigenic polypeptide comprises, or preferably consists of, SEQ ID NO:125 or an amino acid sequence having a sequence identity of at least 90%, preferably of at least 92%, further preferably of at least 95%, and again further preferably of at least 98% amino acid sequence identity with SEQ ID NO:125. In a further very preferred embodiment, said antigenic polypeptide comprises, or preferably consists of, SEQ ID NO:85. In another preferred embodiment, said antigenic polypeptide consists of SEQ ID NO:85. In a further very preferred embodiment, said antigenic polypeptide comprises SEQ ID NO:125. In another preferred embodiment, said antigenic polypeptide consists of SEQ ID NO:125.

In a very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62; or an amino acid sequence having a sequence identity of at least 98% with SEQ ID NO:62, preferably wherein said CMV polypeptide comprises, or preferably consists of, SEQ ID NO:62; (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide comprises an amino acid sequence selected from: (a) SEQ ID NO:85; (b) SEQ ID NO:125 (c) SEQ ID NO:141, and wherein preferably said antigenic polypeptide comprises, preferably consists of SEQ ID NO:125; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of the amino acid sequence of SEQ ID NO:64 or SEQ ID NO:65, again preferably of SEQ ID NO:64.

In a further very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide comprises, preferably consists of SEQ ID NO:125; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:128, SEQ ID NO:132 or SEQ ID NO:139. In a further very preferred embodiment, said chimeric CMV polypeptide consists of SEQ ID NO:128. In a further very preferred embodiment, said chimeric CMV polypeptide consists of SEQ ID NO:132. In a further very preferred embodiment and aspect hereto, the present invention provides said modified virus-like particle (VLP) of cucumber mosaic virus (CMV) for use in method of treating an inflammatory disease, preferably a chronic inflammatory disease in an animal or human. Preferably, said inflammatory disease is selected from RA, MS, Psoriasis, asthma, Crohns, Colitis, COPD, diabetes, neurodermatitis (allergic dermatitis).

In a further very preferred embodiment, the modified virus-like particle (VLP) of cucumber mosaic virus (CMV) comprises at least one fusion protein and further comprises at least one CMV protein, wherein said at least one fusion protein comprises, or preferably consists of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide comprises, preferably consists of SEQ ID NO:125 and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64; and wherein said CMV protein comprises, preferably consists of, a coat protein of CMV, preferably of SEQ ID NO:62, and wherein said CMV protein is optionally modified by a T helper cell epitope, and wherein said T helper cell epitope replaces a N-terminal region of said CMV protein, wherein said N-terminal region of said CMV protein corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:128 and said CMV protein consists of SEQ ID NO:5. In a further very preferred embodiment, said chimeric CMV polypeptide consists of SEQ ID NO:132 and said CMV protein consists of SEQ ID NO:5. In a further very preferred embodiment and aspect hereto, the present invention provides said modified virus-like particle (VLP) of cucumber mosaic virus (CMV) for use in method of treating an inflammatory disease, preferably a chronic inflammatory disease in an animal or human. Preferably, said inflammatory disease is selected from RA, MS, Psoriasis, asthma, Crohns, Colitis, COPD, diabetes, neurodermatitis (allergic dermatitis).

In another very preferred embodiment, said antigenic polypeptide is IL-4, preferably human 11-4. In again a further very preferred embodiment, said antigenic polypeptide comprises, or preferably consists of SEQ ID NO:86. In another preferred embodiment, said antigenic polypeptide consists of SEQ ID NO:86.

In another very preferred embodiment, said antigenic polypeptide is canine IL-4. In again a further very preferred embodiment, said antigenic polypeptide comprises, or preferably consists of SEQ ID NO:87 or an amino acid sequence having a sequence identity of at least 90%, preferably of at least 95%, with SEQ ID NO:87. Preferably, said antigenic polypeptide comprises, or preferably consists of SEQ ID NO:87. In another preferred embodiment, said antigenic polypeptide consists of SEQ ID NO:87.

In another very preferred embodiment, said antigenic polypeptide is feline IL-4. In again a further very preferred embodiment, said antigenic polypeptide comprises, or preferably consists of SEQ ID NO:88 or an amino acid sequence having a sequence identity of at least 90%, preferably of at least 95%, with SEQ ID NO:88. Preferably, said antigenic polypeptide comprises, or preferably consists of SEQ ID NO:88. Preferably, said antigenic polypeptide comprises, or preferably consists of SEQ ID NO:88. In another preferred embodiment, said antigenic polypeptide consists of SEQ ID NO:88.

In another very preferred embodiment, said antigenic polypeptide is IL-13, preferably human IL-13. In again a further very preferred embodiment, said antigenic polypeptide comprises, or preferably consists of SEQ ID NO:89. In another preferred embodiment, said antigenic polypeptide consists of SEQ ID NO:89.

In another very preferred embodiment, said antigenic polypeptide is canine IL-13. In again a further very preferred embodiment, said antigenic polypeptide comprises, or preferably consists of SEQ ID NO:90 or an amino acid sequence having a sequence identity of at least 90%, preferably of at least 95%, with SEQ ID NO:90. Preferably, said antigenic polypeptide comprises, or preferably consists of SEQ ID NO:90. In another preferred embodiment, said antigenic polypeptide consists of SEQ ID NO:90.

In another very preferred embodiment, said antigenic polypeptide is feline IL-13. In again a further very preferred embodiment, said antigenic polypeptide comprises, or preferably consists of SEQ ID NO:91 or an amino acid sequence having a sequence identity of at least 90%, preferably of at least 95%, with SEQ ID NO:91. Preferably, said antigenic polypeptide comprises, or preferably consists of SEQ ID NO:91. In another preferred embodiment, said antigenic polypeptide consists of SEQ ID NO:91.

In another very preferred embodiment, said antigenic polypeptide is equine IL-13. In again a further very preferred embodiment, said antigenic polypeptide comprises, or preferably consists of SEQ ID NO:92 or an amino acid sequence having a sequence identity of at least 90%, preferably of at least 95%, with SEQ ID NO:92. Preferably, said antigenic polypeptide comprises, or preferably consists of SEQ ID NO:92. In another preferred embodiment, said antigenic polypeptide consists of SEQ ID NO:92.

In a further very preferred embodiment, said antigenic polypeptide is TNFα.

In another very preferred embodiment, said antigenic polypeptide is IL-1α, preferably human IL-1α. In again a further very preferred embodiment, said antigenic polypeptide comprises, or preferably consists of SEQ ID NO:93. In another preferred embodiment, said antigenic polypeptide consists of SEQ ID NO:93.

In another very preferred embodiment, said antigenic polypeptide is canine IL-1α. In again a further very preferred embodiment, said antigenic polypeptide comprises, or preferably consists of SEQ ID NO:94 or an amino acid sequence having a sequence identity of at least 90%, preferably of at least 95%, with SEQ ID NO:94. Preferably, said antigenic polypeptide comprises, or preferably consists of SEQ ID NO:94. In another preferred embodiment, said antigenic polypeptide consists of SEQ ID NO:94.

In another very preferred embodiment, said antigenic polypeptide is feline IL-1α. In again a further very preferred embodiment, said antigenic polypeptide comprises, or preferably consists of SEQ ID NO:95 or an amino acid sequence having a sequence identity of at least 90%, preferably of at least 95%, with SEQ ID NO:95. Preferably, said antigenic polypeptide comprises, or preferably consists of SEQ ID NO:95. In another preferred embodiment, said antigenic polypeptide consists of SEQ ID NO:95.

In another very preferred embodiment, said antigenic polypeptide is equine IL-1α. In again a further very preferred embodiment, said antigenic polypeptide comprises, or preferably consists of SEQ ID NO:96 or an amino acid sequence having a sequence identity of at least 90%, preferably of at least 95%, with SEQ ID NO:96. Preferably, said antigenic polypeptide comprises, or preferably consists of SEQ ID NO:96. In another preferred embodiment, said antigenic polypeptide consists of SEQ ID NO:96.

In another very preferred embodiment, said antigenic polypeptide is IL-33, preferably human IL-33. In again a further very preferred embodiment, said antigenic polypeptide comprises, or preferably consists of SEQ ID NO:97. In another preferred embodiment, said antigenic polypeptide consists of SEQ ID NO:97.

In another very preferred embodiment, said antigenic polypeptide is canine IL-33. In again a further very preferred embodiment, said antigenic polypeptide comprises, or preferably consists of SEQ ID NO:98 or an amino acid sequence having a sequence identity of at least 90%, preferably of at least 95%, with SEQ ID NO:98. Preferably, said antigenic polypeptide comprises, or preferably consists of SEQ ID NO:98. In another preferred embodiment, said antigenic polypeptide consists of SEQ ID NO:98.

In another very preferred embodiment, said antigenic polypeptide is feline IL-33. In again a further very preferred embodiment, said antigenic polypeptide comprises, or preferably consists of SEQ ID NO:99 or an amino acid sequence having a sequence identity of at least 90%, preferably of at least 95%, with SEQ ID NO:99. Preferably, said antigenic polypeptide comprises, or preferably consists of SEQ ID NO:99. In another preferred embodiment, said antigenic polypeptide consists of SEQ ID NO:99.

In another very preferred embodiment, said antigenic polypeptide is equine IL-33. In again a further very preferred embodiment, said antigenic polypeptide comprises, or preferably consists of SEQ ID NO:100 or an amino acid sequence having a sequence identity of at least 90%, preferably of at least 95%, with SEQ ID NO:100. Preferably, said antigenic polypeptide comprises, or preferably consists of SEQ ID NO:100. In another preferred embodiment, said antigenic polypeptide consists of SEQ ID NO:100.

In another very preferred embodiment, said antigenic polypeptide is IL-25, preferably human IL-25. In again a further very preferred embodiment, said antigenic polypeptide comprises, or preferably consists of SEQ ID NO:101. In another preferred embodiment, said antigenic polypeptide consists of SEQ ID NO:101.

In another very preferred embodiment, said antigenic polypeptide is canine IL-25. In again a further very preferred embodiment, said antigenic polypeptide comprises, or preferably consists of SEQ ID NO:102 or an amino acid sequence having a sequence identity of at least 90%, preferably of at least 95%, with SEQ ID NO:102. Preferably, said antigenic polypeptide comprises, or preferably consists of SEQ ID NO:102. In another preferred embodiment, said antigenic polypeptide consists of SEQ ID NO:102.

In another very preferred embodiment, said antigenic polypeptide is feline IL-25. In again a further very preferred embodiment, said antigenic polypeptide comprises, or preferably consists of SEQ ID NO:103 or an amino acid sequence having a sequence identity of at least 90%, preferably of at least 95%, with SEQ ID NO:103. Preferably, said antigenic polypeptide comprises, or preferably consists of SEQ ID NO:103. In another preferred embodiment, said antigenic polypeptide consists of SEQ ID NO:103.

In another very preferred embodiment, said antigenic polypeptide is equine IL-25. In again a further very preferred embodiment, said antigenic polypeptide comprises, or preferably consists of SEQ ID NO:104 or an amino acid sequence having a sequence identity of at least 90%, preferably of at least 95%, with SEQ ID NO:104. Preferably, said antigenic polypeptide comprises, or preferably consists of SEQ ID NO:104. In another preferred embodiment, said antigenic polypeptide consists of SEQ ID NO:104.

In a further very preferred embodiment, said antigenic polypeptide is IL-1β, preferably human IL-113. In again a further very preferred embodiment, said antigenic polypeptide comprises, or preferably consists of SEQ ID NO:105. In a further very preferred embodiment, said antigenic polypeptide is canine IL-113. In a very preferred embodiment, said antigenic polypeptide comprises, or preferably consists of, SEQ ID NO:134, SEQ ID NO:143, SEQ ID NO:144 or an amino acid sequence having a sequence identity of at least 90%, preferably of at least 92%, further preferably of at least 95%, and again further preferably of at least 98% amino acid sequence identity with SEQ ID NO:134, SEQ ID NO:143, SEQ ID NO:144. In a further very preferred embodiment, said antigenic polypeptide comprises SEQ ID NO:134, SEQ ID NO:143, SEQ ID NO:144. In a further very preferred embodiment, said antigenic polypeptide consists of SEQ ID NO:134, SEQ ID NO:143, SEQ ID NO:144. In a further very preferred embodiment, said antigenic polypeptide comprises, or preferably consists of, SEQ ID NO:134 or an amino acid sequence having a sequence identity of at least 90%, preferably of at least 92%, further preferably of at least 95%, and again further preferably of at least 98% amino acid sequence identity with SEQ ID NO:134. In a further very preferred embodiment, said antigenic polypeptide comprises, or preferably consists of, SEQ ID NO:135. In another preferred embodiment, said antigenic polypeptide consists of SEQ ID NO:135.

In a very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62; or an amino acid sequence having a sequence identity of at least 98% with SEQ ID NO:62, preferably wherein said CMV polypeptide comprises, or preferably consists of, SEQ ID NO:62; (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide comprises an amino acid sequence selected from: (a) SEQ ID NO:134; (b) SEQ ID NO:143 (c) SEQ ID NO:144, and wherein preferably said antigenic polypeptide comprises, preferably consists of SEQ ID NO:134; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of the amino acid sequence of SEQ ID NO:64 or SEQ ID NO:65, again preferably of SEQ ID NO:64.

In a further very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide comprises, preferably consists of SEQ ID NO:134; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:135. In a further very preferred embodiment and aspect hereto, the present invention provides said modified virus-like particle (VLP) of cucumber mosaic virus (CMV) for use in method of treating an inflammatory disease, preferably a chronic inflammatory disease in an animal or human. Preferably, said inflammatory disease is selected from RA, MS, Psoriasis, asthma, Crohns, Colitis, COPD, diabetes, neurodermatitis (allergic dermatitis).

In a further very preferred embodiment, the modified virus-like particle (VLP) of cucumber mosaic virus (CMV) comprises at least one fusion protein and further comprises at least one CMV protein, wherein said at least one fusion protein comprises, or preferably consists of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide comprises, preferably consists of SEQ ID NO:134 and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64; and wherein said CMV protein comprises, preferably consists of, a coat protein of CMV, preferably of SEQ ID NO:62, and wherein said CMV protein is optionally modified by a T helper cell epitope, and wherein said T helper cell epitope replaces a N-terminal region of said CMV protein, wherein said N-terminal region of said CMV protein corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:135 and said CMV protein consists of SEQ ID NO:5. In a further very preferred embodiment and aspect hereto, the present invention provides said modified virus-like particle (VLP) of cucumber mosaic virus (CMV) for use in method of treating an inflammatory disease.

In a further very preferred embodiment, said antigenic polypeptide is feline IL-113. In again a further very preferred embodiment, said antigenic polypeptide comprises, or preferably consists of SEQ ID NO:145.

In another very preferred embodiment, said antigenic polypeptide is IL-31, preferably human IL-31. In again a further very preferred embodiment, said antigenic polypeptide comprises, or preferably consists of SEQ ID NO:106. In another preferred embodiment, said antigenic polypeptide consists of SEQ ID NO:106.

In another very preferred embodiment, said antigenic polypeptide is canine IL-31. In again a further very preferred embodiment, said antigenic polypeptide comprises, or preferably consists of SEQ ID NO:107 or an amino acid sequence having a sequence identity of at least 90%, preferably of at least 95%, with SEQ ID NO:107. Preferably, said antigenic polypeptide comprises, or preferably consists of SEQ ID NO:107. In another preferred embodiment, said antigenic polypeptide consists of SEQ ID NO:107.

In another very preferred embodiment, said antigenic polypeptide is feline IL-31. In again a further very preferred embodiment, said antigenic polypeptide comprises, or preferably consists of SEQ ID NO:108 or an amino acid sequence having a sequence identity of at least 90%, preferably of at least 95%, with SEQ ID NO:108. Preferably, said antigenic polypeptide comprises, or preferably consists of SEQ ID NO:108. In another preferred embodiment, said antigenic polypeptide consists of SEQ ID NO:108.

In another very preferred embodiment, said antigenic polypeptide is equine IL-31. In again a further very preferred embodiment, said antigenic polypeptide comprises, or preferably consists of SEQ ID NO:109 or an amino acid sequence having a sequence identity of at least 90%, preferably of at least 95%, with SEQ ID NO:109. Preferably, said antigenic polypeptide comprises, or preferably consists of SEQ ID NO:109. In another preferred embodiment, said antigenic polypeptide consists of SEQ ID NO:109.

In another very preferred embodiment, said antigenic polypeptide is thymic stromal lymphopoietin (TLSP), preferably human thymic stromal lymphopoietin (TLSP). In again a further very preferred embodiment, said antigenic polypeptide comprises, or preferably consists of SEQ ID NO:110. In another preferred embodiment, said antigenic polypeptide consists of SEQ ID NO:110.

In another very preferred embodiment, said antigenic polypeptide is canine TLSP. In again a further very preferred embodiment, said antigenic polypeptide comprises, or preferably consists of SEQ ID NO:111 or an amino acid sequence having a sequence identity of at least 90%, preferably of at least 95%, with SEQ ID NO:111. Preferably, said antigenic polypeptide comprises, or preferably consists of SEQ ID NO:111. In another preferred embodiment, said antigenic polypeptide consists of SEQ ID NO:111.

In another very preferred embodiment, said antigenic polypeptide is feline TLSP. In again a further very preferred embodiment, said antigenic polypeptide comprises, or preferably consists of SEQ ID NO:112 or an amino acid sequence having a sequence identity of at least 90%, preferably of at least 95%, with SEQ ID NO:112. Preferably, said antigenic polypeptide comprises, or preferably consists of SEQ ID NO:112. In another preferred embodiment, said antigenic polypeptide consists of SEQ ID NO:112.

In another very preferred embodiment, said antigenic polypeptide is equine TLSP. In again a further very preferred embodiment, said antigenic polypeptide comprises, or preferably consists of SEQ ID NO:113 or an amino acid sequence having a sequence identity of at least 90%, preferably of at least 95%, with SEQ ID NO:113. Preferably, said antigenic polypeptide comprises, or preferably consists of SEQ ID NO:113. In another preferred embodiment, said antigenic polypeptide consists of SEQ ID NO:113.

In again a further very preferred embodiment, said antigenic polypeptide is IgE or a peptide or domain comprised in IgE.

In again a further very preferred embodiment, said antigenic polypeptide is a peptide derived the N-terminus from Aβ-1-42 (SEQ ID NO:114), in particular a fragment of Aβ-1-42 (SEQ ID NO: 114) of at most 7 consecutive amino acids in length, preferably a fragment of Aβ-1-42 (SEQ ID NO: 114) of at most 6 consecutive amino acids in length.

Thus, in a further very preferred embodiment, said antigenic polypeptide is selected from Aβ-1-6 (SEQ ID NO:1), Aβ-1-7 (SEQ ID NO:2), Aβ-3-6 (SEQ ID NO:3), Aβ-1-5 (SEQ ID NO:4), Aβ-2-6 (SEQ ID NO:115), or Aβ-3-7 (SEQ ID NO:116). In a further very preferred embodiment, said antigenic polypeptide is Aβ-1-6 (SEQ ID NO:1). In a further very preferred embodiment, said antigenic polypeptide is Aβ-1-7 (SEQ ID NO:2). In a further very preferred embodiment, said antigenic polypeptide is Aβ-3-6 (SEQ ID NO:3). In a further very preferred embodiment, said antigenic polypeptide is Aβ-1-5 (SEQ ID NO:4). In a further very preferred embodiment, said antigenic polypeptide is Aβ-2-6 (SEQ ID NO:115). In a further very preferred embodiment, said antigenic polypeptide is Aβ-3-7 (SEQ ID NO:116).

In a very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62; or an amino acid sequence having a sequence identity of at least 98% with SEQ ID NO:62, preferably wherein said CMV polypeptide comprises, or preferably consists of, SEQ ID NO:62; (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is Aβ-1-6 (SEQ ID NO:1); and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of the amino acid sequence of SEQ ID NO:64 or SEQ ID NO:65, again preferably of SEQ ID NO:64. In a further very preferred embodiment and aspect hereto, the present invention provides said modified virus-like particle (VLP) of cucumber mosaic virus (CMV) for use in method of treating Alzheimer's disease.

In a further very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is Aβ-1-6 (SEQ ID NO:1); and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:6. In a further very preferred embodiment and aspect hereto, the present invention provides said modified virus-like particle (VLP) of cucumber mosaic virus (CMV) for use in method of treating Alzheimer's disease.

In a very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62; or an amino acid sequence having a sequence identity of at least 98% with SEQ ID NO:62, preferably wherein said CMV polypeptide comprises, or preferably consists of, SEQ ID NO:62; (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is Aβ-1-7 (SEQ ID NO:2); and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of the amino acid sequence of SEQ ID NO:64 or SEQ ID NO:65, again preferably of SEQ ID NO:64. In a further very preferred embodiment and aspect hereto, the present invention provides said modified virus-like particle (VLP) of cucumber mosaic virus (CMV) for use in method of treating Alzheimer's disease.

In a further very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is Aβ-1-7 (SEQ ID NO:2); and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:7. In a further very preferred embodiment and aspect hereto, the present invention provides said modified virus-like particle (VLP) of cucumber mosaic virus (CMV) for use in method of treating Alzheimer's disease.

In a very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62; or an amino acid sequence having a sequence identity of at least 98% with SEQ ID NO:62, preferably wherein said CMV polypeptide comprises, or preferably consists of, SEQ ID NO:62; (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is Aβ-3-6 (SEQ ID NO:3); and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of the amino acid sequence of SEQ ID NO:64 or SEQ ID NO:65, again preferably of SEQ ID NO:64. In a further very preferred embodiment and aspect hereto, the present invention provides said modified virus-like particle (VLP) of cucumber mosaic virus (CMV) for use in method of treating Alzheimer's disease.

In a further very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is Aβ-3-6 (SEQ ID NO:3); and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:8. In a further very preferred embodiment and aspect hereto, the present invention provides said modified virus-like particle (VLP) of cucumber mosaic virus (CMV) for use in method of treating Alzheimer's disease.

In a very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62; or an amino acid sequence having a sequence identity of at least 98% with SEQ ID NO:62, preferably wherein said CMV polypeptide comprises, or preferably consists of, SEQ ID NO:62; (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is Aβ-1-5 (SEQ ID NO:4); and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of the amino acid sequence of SEQ ID NO:64 or SEQ ID NO:65, again preferably of SEQ ID NO:64. In a further very preferred embodiment and aspect hereto, the present invention provides said modified virus-like particle (VLP) of cucumber mosaic virus (CMV) for use in method of treating Alzheimer's disease.

In a further very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is Aβ-1-5 (SEQ ID NO:4); and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:9. In a further very preferred embodiment and aspect hereto, the present invention provides said modified virus-like particle (VLP) of cucumber mosaic virus (CMV) for use in method of treating Alzheimer's disease.

In another very preferred embodiment, said antigenic polypeptide is α-synuclein or a peptide derived from α-synuclein, and wherein preferably said peptide consists of 6 to 14 amino acids, and wherein further preferably said antigenic polypeptide is a peptide derived from α-synuclein selected from any one of SEQ D NO:49, SEQ ID NO:50, SEQ ID NO:51 and SEQ ID NO:117. Further preferred peptides derived from α-synuclein are disclosed in WO 2011/020133, which is incorporated herein by way of reference.

Alpha-synuclein (α-Syn), a small protein with multiple physiological and pathological functions, is one of the dominant proteins found in Lewy Bodies, a pathological hallmark of Lewy body disorders, including Parkinson's disease (PD). More recently, α-Syn has been found in body fluids, including blood and cerebrospinal fluid, and is likely produced by both peripheral tissues and the central nervous system. Exchange of α-Syn between the brain and peripheral tissues could have important pathophysiologic and therapeutic implications (Gardai S J et al., PLoS ONE (2013) 8(8): e71634). The evidence implicating alpha-synuclein (a-syn) in the pathogenesis of Parkinson's Disease (PD) is overwhelming. However, there is not a clear consensus on the manner in which a-syn leads to pathology in PD and other synucleinopathies.

Alpha-synuclein is a major component of Lewy bodies (LBs), and descriptions of a-syn overexpression leading to aggregation are abundant. Human genetic data have demonstrated that missense mutations and multiplications in the a-syn gene cause familial PD. In the case of gene multiplication, increased levels of a-syn protein are presumed to result in a dominant gain-of-function that leads to pathology. While increased levels of a-syn may lead to aggregation and toxicity, research over the past few years has also revealed that elevated a-syn can interfere with the creation, localization, and/or maintenance of vesicle pools (Gardai S J et al., PLoS ONE (2013) 8(8): e71634; and references cited therein.

Thus, in a further very preferred embodiment, said antigenic polypeptide is selected from any one of the sequences selected from SEQ D NO:49, SEQ ID NO:50, SEQ ID NO:51 and SEQ ID NO:117. In a further very preferred embodiment, said antigenic polypeptide is SEQ D NO:49. In a further very preferred embodiment, said antigenic polypeptide is SEQ D NO:50. In a further very preferred embodiment, said antigenic polypeptide is SEQ D NO:51. In a further very preferred embodiment, said antigenic polypeptide is SEQ D NO:117.

In a very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62; or an amino acid sequence having a sequence identity of at least 98% with SEQ ID NO:62, preferably wherein said CMV polypeptide comprises, or preferably consists of, SEQ ID NO:62; (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is SEQ ID NO:49, and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of the amino acid sequence of SEQ ID NO:64 or SEQ ID NO:65, again preferably of SEQ ID NO:64.

In a further very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is SEQ ID NO:49; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:52. In a further very preferred embodiment and aspect hereto, the present invention provides said modified virus-like particle (VLP) of cucumber mosaic virus (CMV) for use in method of treating a disease, disorder or physiological condition, wherein said disease, disorder or physiological condition is selected from a Lewy body disorder, and wherein preferably said disease, disorder or physiological condition is Parkinson's disease.

In a very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62; or an amino acid sequence having a sequence identity of at least 98% with SEQ ID NO:62, preferably wherein said CMV polypeptide comprises, or preferably consists of, SEQ ID NO:62; (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is SEQ ID NO:50; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of the amino acid sequence of SEQ ID NO:64 or SEQ ID NO:65, again preferably of SEQ ID NO:64.

In a further very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is SEQ ID NO:50; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:53. In a further very preferred embodiment and aspect hereto, the present invention provides said modified virus-like particle (VLP) of cucumber mosaic virus (CMV) for use in method of treating a disease, disorder or physiological condition, wherein said disease, disorder or physiological condition is selected from a Lewy body disorder, and wherein preferably said disease, disorder or physiological condition is Parkinson's disease.

In a very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62; or an amino acid sequence having a sequence identity of at least 98% with SEQ ID NO:62, preferably wherein said CMV polypeptide comprises, or preferably consists of, SEQ ID NO:62; (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is SEQ ID NO:51; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of the amino acid sequence of SEQ ID NO:64 or SEQ ID NO:65, again preferably of SEQ ID NO:64.

In a further very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is SEQ ID NO:51; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:54. In a further very preferred embodiment and aspect hereto, the present invention provides said modified virus-like particle (VLP) of cucumber mosaic virus (CMV) for use in method of treating a disease, disorder or physiological condition, wherein said disease, disorder or physiological condition is selected from a Lewy body disorder, and wherein preferably said disease, disorder or physiological condition is Parkinson's disease.

In again a further very preferred embodiment, said antigenic polypeptide is Amylin. In a very preferred embodiment, said antigenic polypeptide is angiotensin I or a peptide derived from angiotensin I. In another very preferred embodiment, said antigenic polypeptide is angiotensin II or a peptide derived from angiotensin II. In a further very preferred embodiment, said antigenic polypeptide is GnRH. In a further very preferred embodiment, said antigenic polypeptide is eotaxin.

In another very preferred embodiment, said antigenic polypeptide is myostatin, preferably cow myostatin. In again a further very preferred embodiment, said antigenic polypeptide comprises, or preferably consists of SEQ ID NO:118 or an amino acid sequence having a sequence identity of at least 90%, preferably of at least 95%, with SEQ ID NO:118. Preferably, said antigenic polypeptide comprises, or preferably consists of SEQ ID NO:118. In another preferred embodiment, said antigenic polypeptide consists of SEQ ID NO:118.

In a further preferred embodiment said antigenic polypeptide is a polypeptide of a parasite, wherein preferably said pathogen is selected from the group consisting of: (a) Toxoplasma spp.; (b) Plasmodium falciparum; (c) Plasmodium vivax; (d) Plasmodium ovale; (e) Plasmodium malariae; (f) Leishmania; (g) Schistosoma and (h) Nematodes. Preferably, said antigenic polypeptide is derived from Plasmodium falciparum or Plasmodium Vivax (SEQ ID NO: 119).

In a further very preferred embodiment, said antigenic polypeptide is derived from Plasmodium falciparum. In a further very preferred embodiment, said antigenic polypeptide derived from Plasmodium falciparum comprises, or preferably consists of, SEQ ID NO:44.

In a very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62; or an amino acid sequence having a sequence identity of at least 98% with SEQ ID NO:62, preferably wherein said CMV polypeptide comprises, or preferably consists of, SEQ ID NO:62; (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide is derived from Plasmodium falciparum, and wherein preferably said antigenic polypeptide derived from Plasmodium falciparum comprises, or preferably consists of, SEQ ID NO:44, and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of the amino acid sequence of SEQ ID NO:64 or SEQ ID NO:65, again preferably of SEQ ID NO:64.

In a further very preferred embodiment, the modified virus-like particle (VLP) of cucumber mosaic virus (CMV) comprises at least one fusion protein comprising, or preferably consisting of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide comprises, or preferably consists of, SEQ ID NO:44; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:46. In a further very preferred embodiment and aspect hereto, the present invention provides said modified virus-like particle (VLP) of cucumber mosaic virus (CMV) for use in method of treating malaria.

In a further very preferred embodiment, the modified virus-like particle (VLP) of cucumber mosaic virus (CMV) comprises at least one fusion protein and further comprises at least one CMV protein, wherein said at least one fusion protein comprises, or preferably consists of, a) a chimeric CMV polypeptide, wherein said chimeric CMV polypeptide comprises, or preferably consists of, (i) a CMV polypeptide, (ii) an antigenic polypeptide and (iii) a T helper cell epitope; and wherein said CMV polypeptide comprises, or preferably consists of, SEQ ID NO:62, (ii) an antigenic polypeptide, wherein said antigenic polypeptide is inserted into said CMV polypeptide, wherein said insertion of said antigenic polypeptide is between amino acid residues of said CMV polypeptide corresponding to amino acid residues of position 84 and position 85 of SEQ ID NO:62; and wherein said antigenic polypeptide comprises, or preferably consists of, SEQ ID NO:44; and wherein said T helper cell epitope replaces a N-terminal region of said CMV polypeptide, wherein said N-terminal region of said CMV polypeptide corresponds to amino acids 2-12 of SEQ ID NO:62, and wherein preferably said T helper cell epitope comprises, preferably consists of SEQ ID NO:64; and wherein said CMV protein comprises, preferably consists of, a coat protein of CMV, preferably of SEQ ID NO:62, and wherein said CMV protein is optionally modified by a T helper cell epitope, and wherein said T helper cell epitope replaces a N-terminal region of said CMV protein, wherein said N-terminal region of said CMV protein corresponds to amino acids 2-12 of SEQ ID NO:62, and wherein preferably said T helper cell epitope comprises, preferably consists of SEQ ID NO:64. In a further very preferred embodiment, said chimeric CMV polypeptide consists of SEQ ID NO:46 and said CMV protein consists of SEQ ID NO:5. In a further very preferred embodiment and aspect hereto, the present invention provides said modified virus-like particle (VLP) of cucumber mosaic virus (CMV) for use in method of treating malaria

In a further preferred embodiment, said antigenic polypeptide is a polypeptide of a bacterium, wherein preferably said bacterium is selected from the group consisting of: (a) Chlamydia (b) Streptococcus; (c) Pneumococcus; (d) Staphylococcus; (e) Salmonella; (f) Mycobacteria; (g) Clostridia (h) Vibrio (i) Yersinia (k) Meningococcus (l) Borelia.

In a further preferred embodiment said antigenic polypeptide is a viral antigen, wherein preferably said viral antigen is a polypeptide selected from the group consisting of: (a) HIV and other retrovirsues; (b) influenza virus, preferably influenza A M2 extracellular domain or HA or HA globular domain; (c) a polypeptide of Hepatitis B virus, preferably preS1; (d) Hepatitis C virus; (e) HPV, preferably HPV16E7 (f) RSV, (g) SARS and other Coronaviruses, (h) Dengue and other Flaviviruses, such as West Nile Virus and Hand Foot and Mouth Disease Virus, (i) Chikungunya and other Alphaviruses. (k) CMV and other Herpesviruses, (l) Rotavirus. In a further very preferred embodiment, said antigenic polypeptide is the derived from RSV. In a further very preferred embodiment, said antigenic polypeptide is the derived from Dengue virus.

In a preferred embodiment, said antigenic polypeptide is the extracellular domain of Influenza A virus M2 protein, or an antigenic fragment thereof. In a very preferred embodiment said antigenic polypeptide comprises or preferably consists of the extracellular domain of the Influenza A virus M2 protein, wherein preferably said extracellular domain of the Influenza A virus M2 protein is SEQ ID NO:120. In another preferred embodiment, said antigenic polypeptide is the globular domain of Influenza virus.

In a further very preferred embodiment, said chimeric CMV polypeptide is selected from the group consisting of SEQ ID NO:6, SEQ ID NO:7, SEQ ID NO:8, SEQ ID NO:9, SEQ ID NO:29, SEQ ID NO:39, SEQ ID NO:46, SEQ ID NO:52, SEQ ID NO:53, or SEQ ID NO:54.

In a further preferred embodiment said modified VLP further comprises at least one immunostimulatory substance. In a very preferred embodiment, said immunostimulatory substance is packaged into the modified VLPs of the invention. In another preferred embodiment, the immunostimulatory substance is mixed with the modified VLPs of the invention. Immunostimulatory substances useful for the invention are generally known in the art and are disclosed, inter alia, in WO2003/024481A2.

In another embodiment of the present invention, said immunostimulatory substance consists of DNA or RNA of non-eukaryotic origin. In a further preferred embodiment said immunostimulatory substance is selected from the group consisting of: (a) immunostimulatory nucleic acid; (b) peptidoglycan; (c) lipopolysaccharide; (d) lipoteichonic acid; (e) imidazoquinoline compound; (f) flagelline; (g) lipoprotein; and (h) any mixtures of at least one substance of (a) to (g). In a further preferred embodiment said immunostimulatory substance is an immunostimulatory nucleic acid, wherein said immunostimulatory nucleic acid is selected from the group consisting of: (a) ribonucleic acids; (b) deoxyribonucleic acids; (c) chimeric nucleic acids; and (d) any mixture of (a), (b) and/or (c). In a further preferred embodiment said immunostimulatory nucleic acid is a ribonucleic acid, and wherein said ribonucleic acid is bacteria derived RNA. In a further preferred embodiment said immunostimulatory nucleic acid is poly(IC) or a derivative thereof. In a further preferred embodiment said immunostimulatory nucleic acid is a deoxyribonucleic acid, wherein said deoxyribonucleic acid is an unmethylated CpG-containing oligonucleotide.

In a very preferred embodiment said immunostimulatory substance is an unmethylated CpG-containing oligonucleotide. In a further preferred embodiment said unmethylated CpG-containing oligonucleotide is an A-type CpG. In a further preferred embodiment said A-type CpG comprises a palindromic sequence. In a further preferred embodiment said palindromic sequence is flanked at its 5′-terminus and at its 3′-terminus by guanosine entities. In a further preferred embodiment said palindromic sequence is flanked at its 5′-terminus by at least 3 and at most 15 guanosine entities, and wherein said palindromic sequence is flanked at its 3′-terminus by at least 3 and at most 15 guanosine entities.

In another preferred embodiment, said immunostimulatory substance is an unmethylated CpG-containing oligonucleotide, and wherein preferably said unmethylated CpG-containing oligonucleotide comprises a palindromic sequence, and wherein further preferably the CpG motif of said unmethylated CpG-containing oligonucleotide is part of a palindromic sequence.

In a further aspect the invention provides the modified virus-like particle of the invention for use as a medicament.

In a further aspect the invention provides a vaccine comprising or alternatively consisting of the modified virus-like particle of the invention. Encompassed are vaccines wherein said modified VLPs comprise any one of the technical features disclosed herein, either alone or in any possible combination. In one embodiment the vaccine further comprises an adjuvant. In a further embodiment the vaccine is devoid of an adjuvant. In a preferred embodiment said vaccine comprises an effective amount of the composition of the invention.

In a further aspect, the invention relates to a pharmaceutical composition comprising: (a) a modified VLP of the invention or a vaccine of the invention; and (b) a pharmaceutically acceptable carrier, diluent and/or excipient. Said diluent includes sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Pharmaceutical compositions of the invention may be in a form which contain salts, buffers, adjuvants, or other substances which are desirable for improving the efficacy of the conjugate. Examples of materials suitable for use in preparation of pharmaceutical compositions are provided in numerous sources including Remington's Pharmaceutical Sciences (Osol, A, ed., Mack Publishing Co., (1990)). In one embodiment said pharmaceutical composition comprises an effective amount of the vaccine of the invention.

A further aspect of the invention is a method of immunization comprising administering a modified VLP of the invention, a vaccine of the invention, or a pharmaceutical composition of the invention to an animal or a human. In a preferred embodiment said method comprises administering a composition of the invention, a vaccine of the invention, or a pharmaceutical composition of the invention to an animal or a human.

A further aspect of the invention is a method of treating or preventing a disease, disorder or physiological condition in an animal said method comprising administering a modified VLP of the invention, a vaccine of the invention, or a pharmaceutical composition of the invention to said animal, wherein preferably said animal can be a human. In a further preferred embodiment said modified VLP, said vaccine, or said pharmaceutical composition is administered to said animal subcutaneously, intravenously, intradermally, intranasally, orally, intranodal or transdermally.

In a further very preferred embodiment, said disease, disorder or physiological condition is selected from the group consisting of an allergy, a cancer, an autoimmune disease, an inflammatory disease, an infectious disease.

In a further very preferred embodiment, said disease, disorder or physiological condition is selected from the group consisting of RA, MS, Psoriasis, asthma, Crohns, Colitis, COPD, diabetes, neurodermatitis (allergic dermatitis), Alzheimer s disease, Parkinson's disease, influenza A virus infection, malaria, RSV infection.

In a further very preferred embodiment, said disease, disorder or physiological condition is an inflammatory disease. In a further very preferred embodiment, said disease, disorder or physiological condition is an inflammatory disease selected from RA, MS, Psoriasis, asthma, Crohns, Colitis, COPD, diabetes, neurodermatitis (allergic dermatitis). In a further very preferred embodiment, said disease, disorder or physiological condition is an infectious disease. In a further very preferred embodiment, said disease, disorder or physiological condition is an inflammatory disease selected from influenza A virus infection, malaria, RSV infection. In a further very preferred embodiment, said disease, disorder or physiological condition is malaria.

In a further very preferred embodiment, said disease, disorder or physiological condition is Alzheimer s disease or Parkinson's disease. In a further very preferred embodiment, said disease, disorder or physiological condition is Alzheimer's disease. In a further very preferred embodiment, said disease, disorder or physiological condition is Parkinson's disease.

### EXAMPLES

### Example 1

### Cloning Fragments of Abeta1-42 into Modified Coat Protein of Cucumber Mosaic Virus (CMV) Generating CMV-Abeta-Chimeric CMV Polypeptides

CMV-Abeta-chimeric CMV polypeptides in accordance with the present invention have been prepared comprising the Abeta protein fragments Abeta1-6 (SEQ ID NO:1), Abeta1-7 (SEQ ID NO:2), Abeta3-6 (SEQ ID NO:3) and Abeta1-5 (SEQ ID NO:4).

These Abeta peptides have been inserted between amino acid residues Ser(88) and Tyr(89) of CMV-Ntt830 (SEQ ID NO:5) which is a very preferred modified CMV coat protein comprising a T helper cell epitope derived from tetanus toxoid. The amino acid sequences of these preferred chimeric CMV polypeptides in accordance with the present invention as follows:

Amino acid sequence of “CMV-Ntt830-Ab16”: SEQ ID NO:6;

Amino acid sequence of “CMV-Ntt830-Ab17”: SEQ ID NO:7;

Amino acid sequence of “CMV-Ntt830-Ab36”: SEQ ID NO:8;

Amino acid sequence of “CMV-Ntt830-Ab15”: SEQ ID NO:9.

The amino acid sequences of these preferred chimeric CMV polypeptides further comprise glycine-serine linkers flanking the introduced Abeta peptides at both termini. All preferred fusions proteins of SEQ ID NO:6 to SEQ ID NO:9 comprise a GGGS-linker (SEQ ID NO:10) directly at the N-terminus of the introduced Abeta peptide and a GGGSGS-linker (SEQ ID NO:11) at the C-terminus of the introduced Abeta peptide.

The corresponding nucleotide sequences of said preferred chimeric CMV polypeptides are as follows:

Nucleic acid sequence of “CMV-Ntt830-Ab16”: SEQ ID NO:12;

Nucleic acid sequence of “CMV-Ntt830-Ab17”: SEQ ID NO:13;

Nucleic acid sequence of “CMV-Ntt830-Ab36”: SEQ ID NO:14;

Nucleic acid sequence of “CMV-Ntt830-Ab15”: SEQ ID NO:15.

For introduction of these Abeta peptides or other antigenic polypetides coding DNA sequences in the corresponding CMV DNA sequence of CMV-Ntt830, BamHI site-containing sequence was introduced at the corresponding position for subsequent cloning. The CMV-Ntt830 coding nucleic acid sequence was prepared as described in Example 3 of WO2016/062720A1 and corresponds to SEQ ID NO:14 of WO2016/062720A1.

The BamHI site was introduced by two-step PCR mutagenesis using below listed oligonucleotides and previously constructed pET-CMV-Ntt830 as a template. The template pET-CMV-Ntt830 was prepared as described in Example 3 of WO2016/062720A1.
1^{st} PCR: Forward—pET-90 primer (anneals pET28a+) (SEQ ID NO:16)
   Reverse—RGSYrev (SEQ ID NO:17)
2nd PCR Forward—RGSYdir (SEQ ID NO:18)
   Reverse—CMV-AgeR (SEQ ID NO:19)

After purification of both PCR products, the next PCR was carried out to join the PCR fragments (5 cycles without primers then 25 cycles using primers pET-90 and CMV-AgeR).

After amplification of the gene, the obtained PCR product was directly cloned into the pTZ57R/T vector (InsTAclone PCR Cloning Kit, Fermentas #K1214). E. coli XL1-Blue cells were used as a host for cloning and plasmid amplification.

To avoid RT-PCR errors, several CMV-Ntt830 gene-containing pTZ57 plasmid clones were sequenced using a BigDye cycle sequencing kit and an ABI Prism 3100 Genetic analyzer (Applied Biosystems). After sequencing, pTZ-plasmid clone without sequence errors containing CMV-Ntt830B gene with introduced BamHI site was cut with NcoI and AgeI enzymes. Then the fragment was subcloned into the NcoI/AgeI sites of the pET-CMV-Ntt830, resulting in the helper vector pET-CMV-Ntt830B.

For introduction of DNA coding for the Amyloid-(beta) peptides, following oligonucleotides were used in PCR reactions (template in all PCRs was pET-CMV-Ntt830):
1st PCR: Forward—C-Ab15 (SEQ ID NO:20)
   Reverse—CMcpR (SEQ ID NO:21)
2nd PCR: Forward—C-Ab16 (SEQ ID NO:22)
   Reverse—CMcpR (SEQ ID NO:21)
3^{rd} PCR: Forward—C-Ab17 (SEQ ID NO:23)
   Reverse—CMcpR (SEQ ID NO:21)
4^{th} PCR: Forward—C-Ab36 (SEQ ID NO:24)
   Reverse—CMcpR (SEQ ID NO:21)

All PCR fragments were directly ligated into pTZ57R/T vector, and corresponding insert-containing plasmid clones were isolated after transformation in E. coli XL1 cells. Several plasmid clones containing PCR product DNA were sequenced using a BigDye cycle sequencing kit and an ABI Prism 3100 Genetic Analyser (Applied Biosystems). After sequencing, the Ab fragment-containing CMV 3′end fragments were ligated into pET-CMV-Ntt830B helper vector, using sites BamHI and HindIII. The correct clones were selected after BamHI/HindIII restriction enzyme tests.

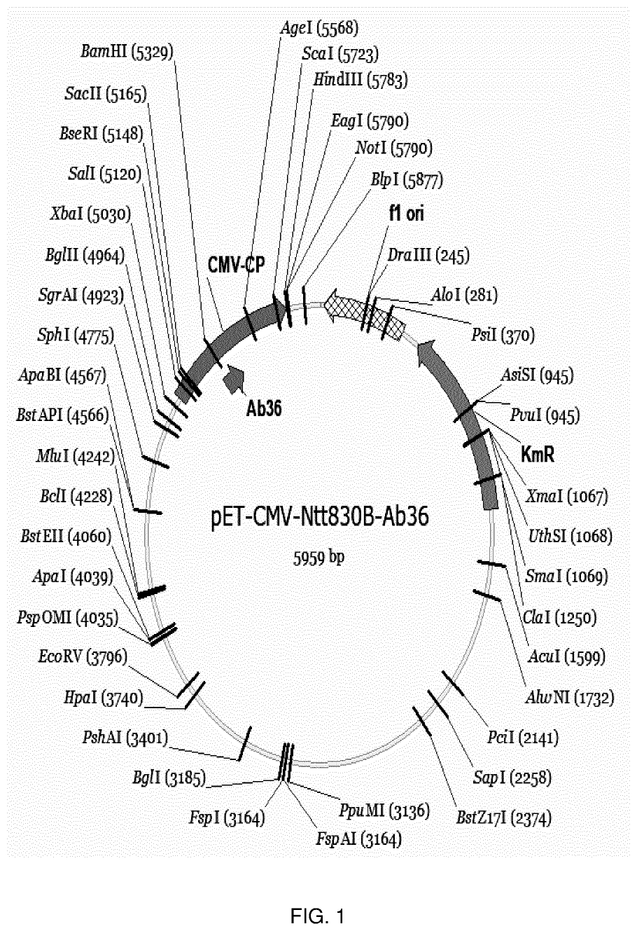

Further, plasmid clones pET-CMV-Ntt830B-Ab15, pET-CMV-Ntt830B-Ab16, pET-CMV-Ntt830B-Ab17 and pET-CMV-Ntt830B-Ab36 were used for transformation of E. coli C2566 cells. The plasmid map of pET-CMV-Ntt830B-Ab36 is exemplarily shown in FIG. 1.

### Example 2

### Expression of the CMV-Abeta-Chimeric CMV Polypeptides Leading to CMV-Abeta VLPs

For isolation of the CMV-Abeta VLPs, namely CMV-Ntt830-Ab16 VLP, CMV-Ntt830-Ab17 VLP, CMV-Ntt830-Ab36 VLP or CMV-Ntt830-Ab15 VLP, E. coli C2566 (New England Biolabs, USA) competent cells were transformed with the corresponding plasmids pET-CMV-Ntt830-Ab15, pET-CMV-Ntt830-Ab16, pET-CMV-Ntt830-Ab17 and pET-CMV-Ntt830-Ab36.

After selection of clones with the highest expression level of target protein, E. coli cultures were grown in 2TY (Trypton 1.6%, yeast extract 1%, 0.5% NaCl, 0.1% glucose) medium containing kanamycin (25 mg/1) on a rotary shaker at 30° C. to the OD(600) value of 0.8-1.0. Then, the cells were induced with 0.2 mM IPTG, and the medium was supplemented with 5 mM MgCl₂. Incubation was continued on the rotary shaker at 20° C. for 18 h. The resulting biomass was collected by low-speed centrifugation and was frozen at −20° C.

The purification of the CMV-Abeta VLPs includes the following steps:

1) suspend 3 g biomass in 20 ml of 50 mM Na citrate, 5 mM Na borate, 5 mM EDTA, 5 mM mercaptoethanol, pH 9.0, treat the suspension with ultrasound (Hielscher sonicator UP200S, 16 min, amplitude 70%, cycle 0.5);

2) Centrifuge the lysate at 11000 rpm for 20 min, at +4° C.;

3) Prepare sucrose gradient (20-60%) in 35 ml tubes, in buffer containing 50 mM Na citrate, 5 mM Na borate, 2 mM EDTA, 0.5% TX-100;

4) Overlay 5 ml of the VLP sample over the sucrose gradient;

5) Centrifuge 6 h using SW32 rotor, Beckman (25000 rpm, at +18° C.).

6) Divide the content of each gradient tube in 6 ml fractions. Pool corresponding fractions;

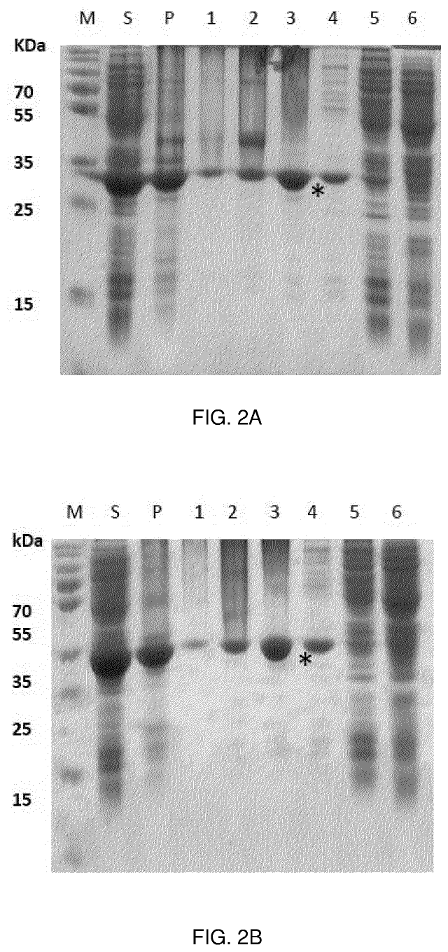

7) Analyse gradient fractions on SDS-PAGE (FIG. 2A, FIG. 2B, FIG. 2C, FIG. 2D).

8) SDS-PAGE analysis suggest the presence of VLPs in 3rd sucrose gradient fraction. Dilute 24 ml of 3rd fraction with 24 ml of buffer (5 mM Na borate, 2 mM EDTA, pH 9.0);

9) Collect the VLPs by ultracentrifugation using rotor Type 70 (Beckman Optima, L100XP ultracentrifuge; 4 h, at 50 000 rpm, 5° C.);

10) Solubilize the pellet in 3 ml of 5 mM Na borate, 2 mM EDTA, pH 9.0;

11) Overly the VLP suspension on the top of 20% sucrose “cushion” (in 5 mM Na borate, 2 mM EDTA, pH 9.0 buffer);

12) Collect the VLPs by ultracentrifugation using rotor TLA100.3 (Beckman; 1 h, at 72000 rpm, 5° C.);

13) Solubilize the pellet in 2 ml of 5 mM Na borate, 2 mM EDTA, pH 9.0, ON, 4° C.;

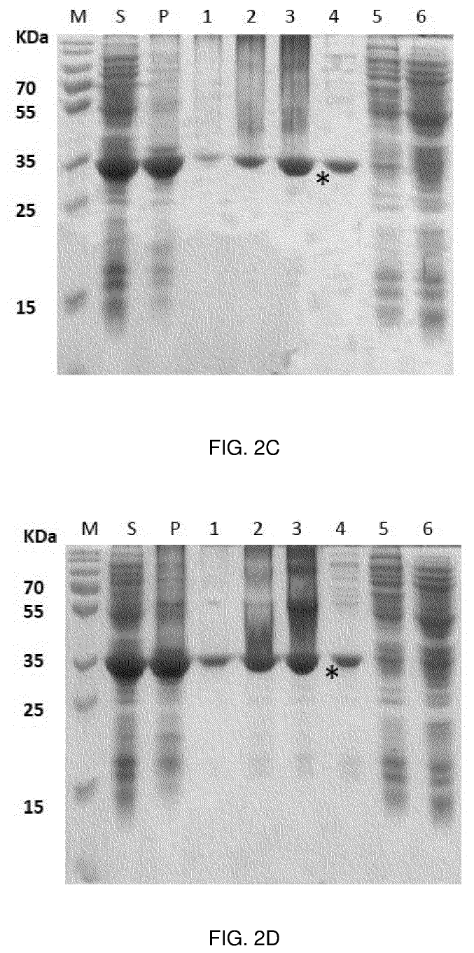

14) Analyse the VLPs under EM (FIG. 3A, FIG. 3B, FIG. 3C, FIG. 3D).

As shown in FIG. 2A, FIG. 2B, FIG. 2C, FIG. 2D, the four CMV-Abeta VLPs can be successfully expressed in E. coli cells and significant part obtained can be in soluble fraction. Moreover, these proteins are found directly in E. coli cell extracts in the form of isometric VLPs, as demonstrated by sucrose gradient analysis (FIG. 2A-2D), and electron-microscopy analysis (FIG. 3A-3D).

### Example 3

### Monoclonal Antibody with Variable Region Sequence of Aducanumab Recognizes CMV-Abeta VLPs

ELISA plates were coated with Aβ₁₋₄₂, CMV-Ntt830-Ab36 VLP or CMV-Ntt830 VLP and binding of a recombinant antibody with the variable regions exhibiting the sequence of Aducanumab was tested by ELISA.

ELISA:

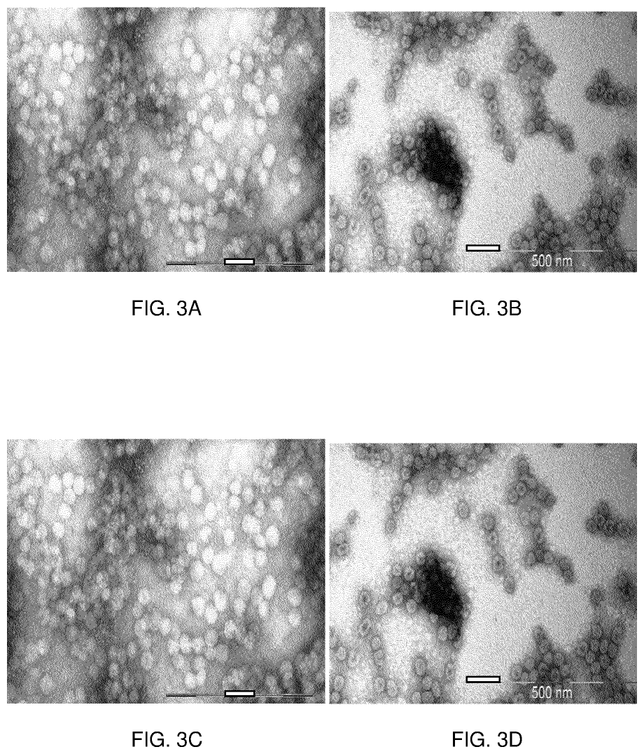

ELISA plates (Nunc Immuno MaxiSorp, Rochester, N.Y.) were coated overnight at 4° C. with 100 μl of Aβ₁₋₄₂, CMV-Ntt830-Ab36 VLP or CMV-Ntt830 VLP (1 μg/ml) in PBS, pH 7.4. In order to avoid unspecific binding the ELISA plates were blocked with 200 μl of 2% BSA in PBST and incubated for 2 hours at RT. Supernatant of cells expressing monoclonal antibodies with the variable region sequence of Aducanumab were transferred onto the coated plates. After 2 hours of incubation at RT, the ELISA plates were washed 5× with 200 μl of PB ST. Binding of serum antibodies was detected by horse-radish peroxidase-conjugated goat anti-human IgG (Jackson ImmunoResearch). The detection antibody was diluted 1:1000 in 2% BSA/PBST and a volume of 100 μl per sample was transferred. The plates were incubated for 1 hour at RT. ELISA plates were washed as described before. Prior washing the substrate solution was prepared. To this end, 1 tablet (10 mg) of OPD (1,2-Phenylenediamine dihydrochloride) and 9 μl of 30% H₂O₂ was dissolved in 25 ml citric acid buffer (0.066 M Na₂HPO₄, 0.035 M citric acid, pH 5.0). A volume of 100 μl of the substrate solution was pipetted onto the plates and exactly incubated for 7 minutes at RT. To stop the reaction 50 μl of stop solution (5% H₂SO₄ in H₂O) was directly pipetted onto the plates. Absorbance readings at 450 nm of the 1, 2-Phenylenediamine dihydrochloride color reaction were analyzed. FIG. 4 shows binding of monoclonal antibodies with the variable region sequence of Aducanumab to the CMV-Ntt830-Ab eta VLP.

### Example 4

### Immunization of Mice with CMV-Abeta VLP

Groups of four female Balb/c mice were either immunized with CMV-Ntt830-Ab16 VLP, CMV-Ntt830-Ab17 VLP or CMV-Ntt830-Ab36 VLP. The VLPs were formulated in 150 mM PBS, pH 7.4 and 150 μl and were injected i.v. on day 0 at 30 ug. Mice were bled on days 0 (pre-immune) and 14, and sera were analyzed using Abeta1-42 coated ELISA plates. Antibodies induced by CMV-Ntt830-Ab36 VLP were further analysed by immunohistochemistry on brain sections from Alzheimer's patients.

ELISA:

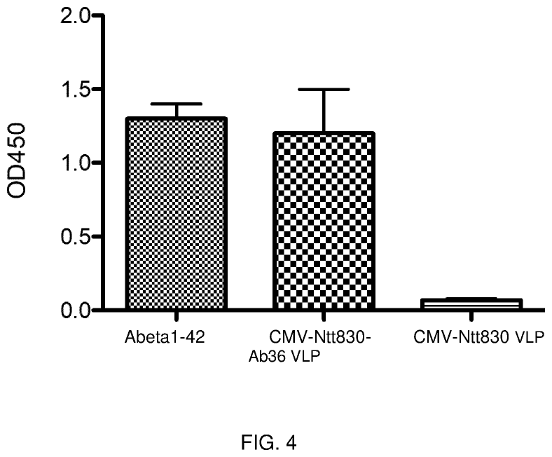

The antibody response in mouse sera were analyzed at the indicated time. For determination of Abeta1-42 specific antibodies, ELISA plates (Nunc Immuno MaxiSorp, Rochester, N.Y.) were coated overnight at 4° C. with 100 μl of Abeta1-42 (1 μg/ml) in PBS, pH 7.4. In order to avoid unspecific binding the ELISA plates were blocked with 200 μl of 2% BSA in PBST and incubated for 2 hours at RT. The serum samples were diluted in 2% BSA/PBST. Pre-diluted sera were transferred onto the coated plates and further serial diluted to obtain antibody titers based on OD50 calculation. After 2 hours of incubation at RT, the ELISA plates were washed 5× with 200 μl of PBST. Binding of serum antibodies was detected by horse-radish peroxidase-conjugated goat anti-mouse IgG (Jackson ImmunoResearch). The detection antibody was diluted 1:1000 in 2% BSA/PBST and a volume of 100 μl per sample was transferred. The plates were incubated for 1 hour at RT. ELISA plates were washed as described before. Prior washing the substrate solution was prepared. To this end, 1 tablet (10 mg) of OPD (1,2-Phenylenediamine dihydrochloride) and 9 μl of 30% H₂O₂ was dissolved in 25 ml citric acid buffer (0.066 M Na₂HPO₄, 0.035 M citric acid, pH 5.0). A volume of 100 μl of the substrate solution was pipetted onto the plates and exactly incubated for 7 minutes at RT. To stop the reaction 50 μl of stop solution (5% H₂SO₄ in H₂O) was directly pipetted onto the plates. Absorbance readings at 450 nm of the 1, 2-Phenylenediamine dihydrochloride color reaction were analyzed. FIG. 5A shows antibodies specific for CMV-Ntt830-Ab16 VLP, CMV-Ntt830-Ab17 VLP or CMV-Ntt830-Ab36 VLP.

Immunohistochemistry:

Sections of paraffin embedded brain (hippocampus) tissue of an Alzheimer's patient were prepared with a microtome. Slized were mounted on slides and endogenous Peroxidase was blocked by incubation with 3% H₂O₂ for 10 min. Slides were then washed with PBS-Tween followed by PBS only 3× for 5 minutes. Slides were blocked with PBS plus oat serum 3%, Casein 0.5%, NaN₃ 0.1% for 30 minutes at RT. Slides were incubated with 1:50 diluted serum from CMV-Ntt830-Ab36 VLP immunized mice at for degrees for one hours. Slides were washed with PBS-Tween followed by PBS only was three times for 5 minutes. Slides were incubated then with secondary antibody goat anti mouse IgG-HRP (#161) 1:1000 for 2 h at RT, washed by PBS-Tween followed by PBS only wash 3×5 for 5 minutes. Bound antibody was visualized by DAB substrate (using Kit abcam ab64238) followed by wash by water. Counter staining was performed with Haematoxilin for 30 seconds followed by wash in water for 2 minutes. FIG. 5B shows staining of plaques by immune serum induced by CMV-Ntt830-Ab36 VLP.

### Example 5

### Cloning of a Modified Coat Protein of CMV Comprising Ara-h202

To obtain mosaic VLPs containing antigens from single plasmid system in accordance with the present invention, the step of the construction was the insertion of CMV-Ntt830 gene in the polylinker of pETDuet-1 (Novagen) under the second T7 promotor. Hereto, the CMV-Ntt830 nucleic acid sequence was prepared as described in Example 3 of WO2016/062720A1 and corresponds to SEQ ID NO:14 of WO2016/062720A1. For CMV-structural gene with corresponding restriction sites for cloning, said CMV-Ntt830 gene was amplified in PCR reaction using following the oligonucleotides:

Forward: CM-830NdeF (SEQ ID NO:25)

Reverse: CM-cpR (SEQ ID NO:26)

After amplification of the gene, the corresponding PCR product was directly cloned into the pTZ57R/T vector (InsTAclone PCR Cloning Kit, Fermentas #K1214). E. coli XL1-Blue cells were used as a host for cloning and plasmid amplification. To avoid RT-PCR errors, several CMV-Ntt830 gene-containing pTZ57 plasmid clones were sequenced using a BigDye cycle sequencing kit and an ABI Prism 3100 Genetic analyzer (Applied Biosystems). After sequencing, pTZ-plasmid clone containing CMV-Ntt830 gene without sequence errors was cut with HindIII enzyme, treated with Klenow enzyme and finally with NdeI restrictase. Then the fragment was subcloned into the NdeI/EcoRV sites of the pETDuet-1, resulting in the helper vector pETDu-CMV-Ntt830.

For the insertion of Ara-h202 protein coding DNA sequence in the CMV-Ntt830 nucleic acid, BamHI site-containing 15 and 10 amino acid long Gly-Ser linker coding sequence were introduced such as to generate said insertion between positions corresponding to Ser(88) and Tyr(89) of SEQ ID NO:5 of CMV-Ntt830. The amino acid sequence of Ara-h202 protein is described in SEQ ID NO:27, whereas the corresponding DNA sequence is described in SEQ ID NO:28.

The amino acid sequence of this preferred chimeric CMV polypeptide in accordance with the present invention referred as “CMV-Ntt830-Arah202” is SEQ ID NO:29. This amino acid sequence of this preferred chimeric CMV polypeptide comprise glycine-serine linkers flanking the introduced Ara-h202 protein at both termini, namely a 15 amino acid long GS-linker (SEQ ID NO:30) directly at the N-terminus of the introduced Ara-h202 protein and a 10 amino acid long GS-linker (SEQ ID NO:31) at the C-terminus of the introduced Ara-h202 protein. The corresponding nucleotide sequence of this preferred chimeric CMV polypeptide CMV-Ntt830-Arah202 is described in SEQ ID NO:32.

First, CMV-Ntt830 gene fragments and Ara-h202 coding sequence (BamHI flanked, without “start” and “stop” codons) were amplified in PCR reactions, using the following oligonucleotides:

1^{st} PCR of CMV gene 5′-end:

Forward: 830-NcoF (SEQ ID NO:33)

Reverse: C-5xg4s-R (SEQ ID NO:34)

Template: pET-CMV-Ntt830 was used, which was prepared as described in Example 3 of WO2016/062720A1

2^{nd} PCR of CMV gene 3′-end:

Forward: C-5xg4s-F (SEQ ID NO:35)

Reverse: CMcpR (SEQ ID NO:21)

Template: pET-CMV-Ntt830 was used, which was prepared as described in Example 3 of WO2016/062720A1

3^{rd} PCR of Arah202:

Forward: Ara-BamF2 (SEQ ID NO:36)

Reverse: Ara-BamR2 (SEQ ID NO:37)

Template: gene-synthesized Ara-h202 gene in pUCIDT plasmid, prepared as described in Example 13 of WO 2017/186808A1.

All PCR fragments were directly ligated into pTZ57R/T vector, and corresponding insert-containing plasmid clones were isolated after transformation in E. coli XL1 cells. To avoid PCR errors, several plasmid clones containing PCR product DNA were sequenced using a BigDye cycle sequencing kit and an ABI Prism 3100 Genetic Analyser (Applied Biosystems). After sequencing, the fragment of CMV 3′end was ligated into pTZ57-CMV-5-end vector, in sites BamHI and HindIII. As a result, the helper plasmid pTZ-CMVB2 was obtained, which contains a GlySer linker and BamHI sites for further subcloning of Ara-h202 coding sequence. As a next step, Ara-h202 fragment after partial BamHI treatment was subloned into pTZ-CMVB2 BamHI site. Correct clones, containing Ara-h202 insert in correct orientation, were found in “colony PCR” reaction, using primers Ara-BamF2/CMcpR. Plasmid clones with positive PCR signal were resequenced. Sequencing results of pTZ-CMVB2-Ara-h202 plasmid clone confirmed the presence of Ara-h202 gene fused with CMV.

To construct the expression vector, the CMVB2-Arah202 insert was excised from helper plasmid using NcoI and HindIII enzymes and subcloned in constructed helper vector pETDu-CMV-Ntt830. The plasmid map of the resulting pETDu-CMVB2xArah202-CMV-tt is shown in FIG. 6.

### Example 6

### Expression of a Mosaic Particle Containing Modified Coat Protein of CMV and in-Fused Ara-h202 (CMV-M-Arah202)

For isolation of mosaic CMV-Arah202 VLPs, E. coli C2566 (New England Biolabs, USA) competent cells were transformed with the plasmid pETDu-CMVB2xArah202-CMV-tt.

After selection of clones with the highest expression level of target protein, E. coli cultures were grown in 2TY (Trypton 1.6%, yeast extract 1%, 0.5% NaCl, 0.1% glucose) medium containing ampicillin (100 mg/1) on a rotary shaker at 30° C. to the OD(600) value of 0.8-1.0. Then, the cells were induced with 0.2 mM IPTG, and the medium was supplemented with 5 mM MgCl₂. Incubation was continued on the rotary shaker at 20° C. for 18 h. The resulting biomass was collected by low-speed centrifugation and was frozen at −20° C. Biomass output—approx. 12 g wet biomass/1 culture, OD(600) was 6.8 at the end of cultivation.

The purification of mosaic VLPs comprising CMV-Ntt830-Arah202 and unmodified CMV-Ntt830 proteins (said mosaic VLPs referred to as “CMV-M-Arah202”) in accordance with the present invention, includes the following steps:

1) suspend 6 g biomass in 20 ml of 50 mM Na citrate, 5 mM Na borate, 5 mM EDTA, 5 mM mercaptoethanol, pH 9.0, treat the suspension with ultrasound (Hielscher sonicator UP200S, 16 min, amplitude 70%, cycle 0.5);

2) Centrifuge the lysate at 11000 rpm for 20 min, at +4° C.;

3) Prepare sucrose gradient (20-60%) in 35 ml tubes, in buffer containing 50 mM Na citrate, 5 mM Na borate, 2 mM EDTA, 0.5% Tx-100;

4) Overlay 5 ml of the VLP sample over the sucrose gradient. Prepare 4 tubes;

5) Centrifuge 6 h using SW32 rotor, Beckman (25000 rpm, at +18° C.).

6) Divide the content of each gradient tube in 6 ml fractions. Pool corresponding fractions;

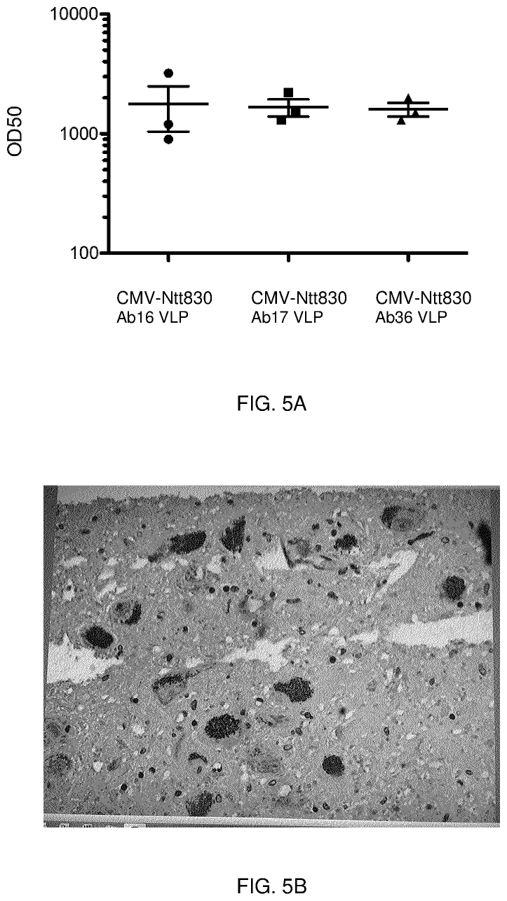

7) Analyse gradient fractions on SDS-PAGE and Western blot (FIG. 7).

8) SDS-PAGE analysis suggest the presence of mosaic VLPs in 3rd sucrose gradient fraction. Dilute 24 ml of 3rd fraction with 24 ml of buffer (5 mM Na borate, 2 mM EDTA, pH 9.0);

9) Collect the VLPs by ultracentrifugation using rotor Type 70 (Beckman Optima, L100XP ultracentrifuge; 4 h, at 50 000 rpm, 5° C.);

10) Solubilize the pellet in 3 ml of 5 mM Na borate, 2 mM EDTA, pH 9.0;

11) Overly the VLP suspension on the top of 20% sucrose “cushion” (in 5 mM Na borate, 2 mM EDTA, pH 9.0 buffer);

12) Collect the VLPs by ultracentrifugation using rotor TLA100.3 (Beckman; 1 h, at 72000 rpm, 5° C.);

13) Solubilize the pellet in 2 ml of 5 mM Na borate, 2 mM EDTA, pH 9.0, ON, 4° C.;

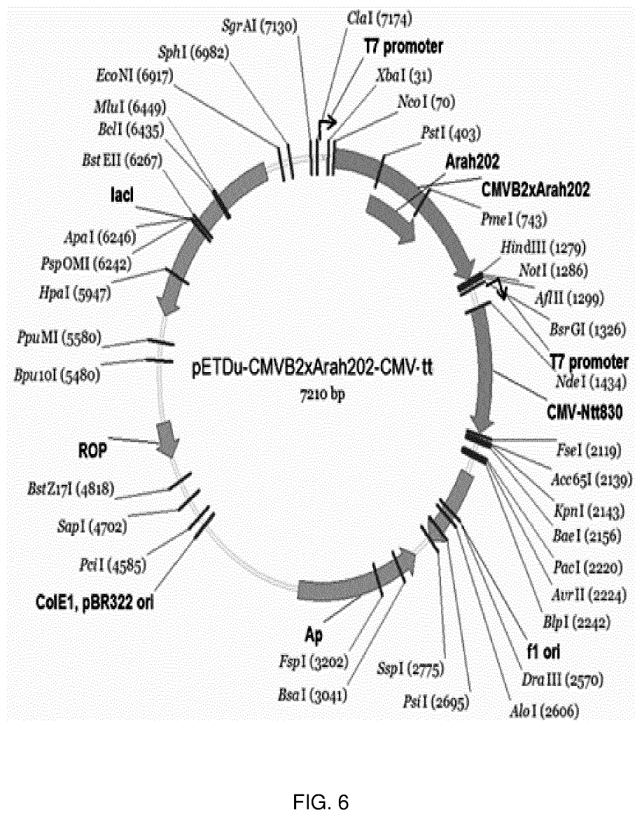

14) Analyse the VLPs after purification on SDS-PAGE gel (FIG. 7) as well as under EM (FIG. 8).

### Example 7

### Immunization of Mice with Mosaic Particle CMV-M-Arah202

Groups of three female Balb/c mice were immunized s.c. with a total Ara-h202 amount of 10 μg either as VLPs comprising CMV-Ntt830-Arah202 and unmodified CMV-Ntt830 proteins (CMV-M-Arah202) or with Ara-h202 in free form. VLPs were formulated in 150 mM PBS, pH 7.4 and 10 ug were injected s.c. in a volume of 150 ul. Mice were bled 14 days after immunization and all immune sera were tested by ELISA against recombinant Ara-h202.

ELISA:

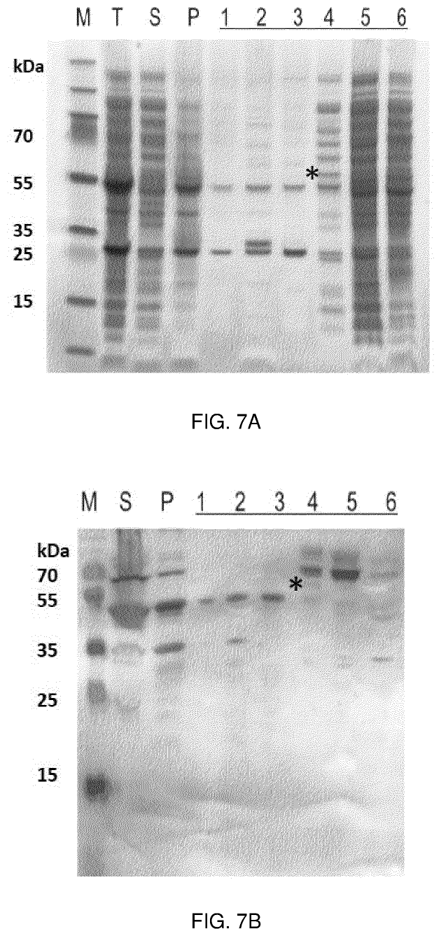

The antibody response in mouse sera were analyzed at the indicated time. For determination of Ara-h202 specific antibody titers, ELISA plates (Nunc Immuno MaxiSorp, Rochester, N.Y.) were coated overnight at 4° C. with 100 μl of Ara-h2 purified out of peanut extracts (1 μg/ml) in PBS. In order to avoid unspecific binding the ELISA plates were blocked with 200 μl of 2% BSA in PBST and incubated for 2 hours at RT. The serum samples were diluted in 2% BSA/PBST. Pre-diluted sera were transferred onto the coated plates and further serial diluted to obtain antibody titers based on OD50 calculation. After 2 hours of incubation at RT, the ELISA plates were washed 5× with 200 μl of PBST. Binding of serum antibodies was detected by horse-radish peroxidase-conjugated goat anti-mouse IgG (Jackson ImmunoResearch). The detection antibody was diluted 1:1000 in 2% BSA/PBST and a volume of 100 μl per sample was transferred. The plates were incubated for 1 hour at RT. ELISA plates were washed as described before. Prior washing the substrate solution was prepared. To this end, 1 tablet (10 mg) of OPD (1,2-Phenylenediamine dihydrochloride) and 9 μl of 30% H₂O₂ was dissolved in 25 ml citric acid buffer (0.066 M Na₂HPO₄, 0.035 M citric acid, pH 5.0). A volume of 100 μl of the substrate solution was pipetted onto the plates and exactly incubated for 7 minutes at RT. To stop the reaction 50 μl of stop solution (5% H₂SO₄ in H₂O) was directly pipetted onto the plates. Absorbance readings at 450 nm of the 1, 2-Phenylenediamine dihydrochloride color reaction were analyzed. FIG. 9 shows antibodies specific for Ara-h2.

For determination of Ara h202 IgG, 96-well Nunc Maxisorp™ ELISA plates (Thermo Fisher Scientific, Waltham, Mass., USA) were coated with 2 μg/ml Ara h2 in PBS buffer at 4° C. overnight. After blocking with PBS/0.15% Casein solution for 2 hours, plates were washed five times with PBS/0.05% Tween. Serial dilutions of sera were added to the plates and incubated for 2 hours at 4° C. Plates were then washed five times with PBS/0.05% Tween (PBST). Thereafter, HRP0-labeled goat anti-mouse IgG (The Jackson Laboratory, Bar Harbor, Me., USA) antibodies were incubated at 4° C. for 1 hour. ELISAs were developed with TMB (3,30,5,50-tetramethyl-benzidine) and H₂O₂ and stopped with 1 mol/L sulfuric acid. Optical densities were measured at 450 nm. Half-maximal antibody titers are defined as the reciprocal of the dilution leading to half of the OD measured at saturation. FIG. 9 shows antibodies specific for Ara-h202.

### Example 8

### Immunization with CMV-M-Arah202 Protects Against Anaphylactic Reaction

Sensitization and Vaccination

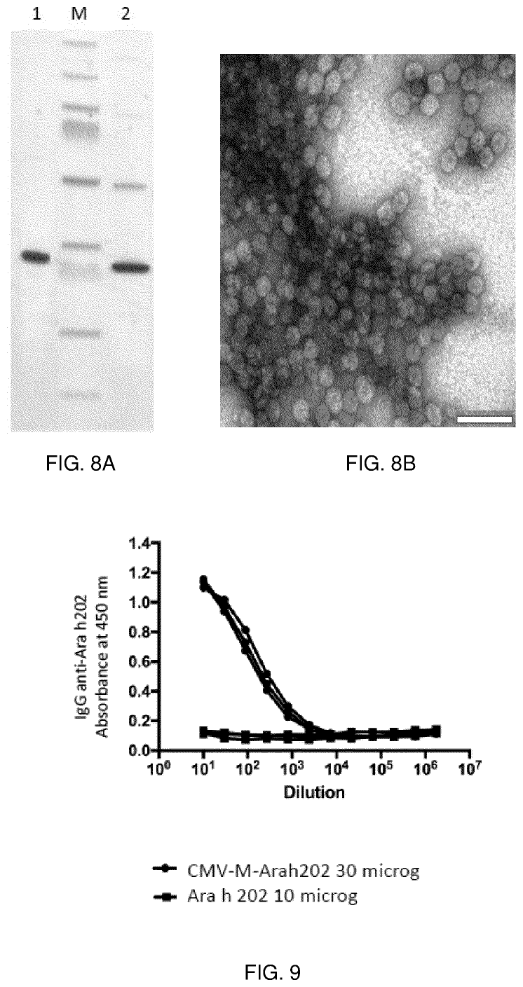

Mice were sensitized to peanut allergens by i.p. injection of peanut extract formulated in 200 μl Alum on days 0 and 7. Mice were then vaccinated with CMV-M-Arah202 or CMV-Ntt830 VLP for the control group (30 ug in 200 ul PBS at day 21). FIG. 10A shows the experimental design to investigate the protective effect of vaccination with CMV-M-Arah202 against allergic systemic and local reaction.

Systemic and Local Challenge

Challenge was performed with peanut extract 20 ug i.v. or via skin prick test (180 ug/20 ul PBS) on the ear of sensitized and vaccinated BALB/c. Systemic and local allergic reactions were determined by temperature drop (FIG. 10B; CMV-Ntt830 VLP abbreviated as CMV for simplicity reasons) or fluid tissue extravasation (diameter of the dot, FIG. 10C, CMV-Ntt830 VLP abbreviated as CMV for simplicity reasons), respectively. Graphs are representative for 2 independent experiments. Mean values+/−SEM of 5 mice per group are shown. Anaphylaxis curves were analyzed by two-way-Anova test. Skin prick tests were analyzed by two-tailed Student's t-test.

### Example 9

### Construction and Expression of a Mosaic Particle Containing Modified Coat Protein of CMV and In-Fused FEL D 1 (CMV-M-Fel)

First, the coding sequence for the preferred chimeric CMV polypeptide in accordance with the present invention referred to as “CMV-Ntt830-Feld12” is prepared.

CMV-Ntt830-Feld12 comprises the Fel d1 protein of SEQ ID NO:38 which corresponds to a fusion protein of chain 1 and chain 2 of Fel d1 with a 15 amino acid GS-linker linking said chain 1 with said chain 2. The construct of SEQ ID NO:38 is described in Example 7 of WO 2017/042241. Further within CMV-Ntt830-Feld12, said is Fel d1 protein of SEQ ID NO:38 is flanked by glycine-serine linkers, in detail, said Fel d1 protein of SEQ ID NO:38 is directly flanked at its N-terminus by the 15 amino acid long GS-linker of SEQ ID NO:30 and directly flanked at its C-terminus by the 10 amino acid long GS-linker of SEQ ID NO:31. Further, said aforementioned described entire construct, i.e. the construct of SEQ ID NO:38 flanked by the described glycine-serine linkers, is inserted between positions corresponding to Ser(88) and Tyr(89) of SEQ ID NO:5 of CMV-Ntt830 leading to CMV-Ntt830-Feld12.

The amino acid sequence of this preferred chimeric CMV polypeptide in accordance with the present invention referred to as “CMV-Ntt830-Feld12” is SEQ ID NO:39. The corresponding nucleotide sequence of this preferred chimeric CMV polypeptide CMV-Ntt830-Feld12 is described in SEQ ID NO:40.

The CMV-Ntt830-Feld12 gene was analogously prepared as described in Example 7 of WO 2017/042241, the entirety of its disclosure is incorporated herein by way of reference. To obtain said CMV-Ntt830-Feld12 gene, first the Fel d1 corresponding gene was amplified with following oligonucleotides by PCR:

Forward: FG4S-BamF (SEQ ID NO:41)

Reverse: FG4S-BamR (SEQ ID NO:42)

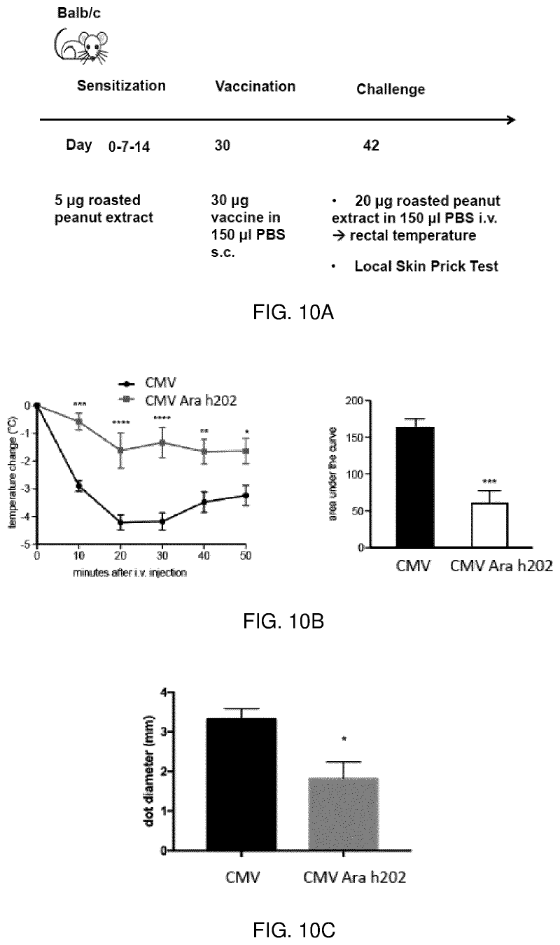

PCR fragment containing flanking Gly-Ser linkers and BamHI sites at both ends was directly ligated into pTZ57R/T vector, and corresponding insert-containing plasmid clones were isolated after transformation in E. coli XL1 cells. To find insert without PCR errors, several plasmid clones containing PCR product DNA were sequenced using a BigDye cycle sequencing kit and an ABI Prism 3100 Genetic Analyser (Applied Biosystems). After sequencing, the correct Fel d1 fragment was ligated into the helper vector pET-CMV-Ntt830B, in site BamHI. Correct clones, containing Fel d1 insert in correct orientation, were found in “colony PCR” reaction, using primers FG4S-BamF/CMcpR. Plasmid clones with positive PCR signal were resequenced and correct clones chosen for further cloning. As a result, the helper plasmid pET-CMVB-Feld1 was obtained. As a next step, CMVB-Feld1 fragment after NcoI/HindIII treatment was subcloned into helper vector pETDu-CMV-Ntt830 (see Example 5). To construct the expression vector without Amp resistance gene, the whole cassette containing CMV-Ntt830-Feld12 fusion and unmodified CMV-Ntt830 gene was excised and subcloned into NcoI/XhoI site of pET28a+(Novagen), resulting in expression vector pET28-CMVBxFeld1-CMVtt. Plasmid map is shown in FIG. 11.

The isolation of mosaic CMV-Feld1 VLPs, i.e. mosaic VLPs comprising CMV-Ntt830-Feld12 and unmodified CMV-Ntt830 proteins (referred to as “CMV-M-Fel”) in accordance with the present invention, includes the following steps:

E. coli C2566 (New England Biolabs, USA) competent cells were transformed with the plasmid pET28-CMVBxFeld1-CMVtt.

After selection of clones with the highest expression level of target protein, E. coli cultures were grown in 2TY (Trypton 1.6%, yeast extract 1%, 0.5% NaCl, 0.1% glucose) medium containing kanamycin (25 mg/1) on a rotary shaker at 30° C. to the OD(600) value of 0.8-1.0. Then, the cells were induced with 0.2 mM IPTG, and the medium was supplemented with 5 mM MgCl₂. Incubation was continued on the rotary shaker at 20° C. for 18 h. The resulting biomass was collected by low-speed centrifugation and was frozen at −20° C.

The purification of mosaic CMV-M-Fel VLPs includes following steps:

1) suspend 6 g biomass in 20 ml of 50 mM Na citrate, 5 mM Na borate, 5 mM EDTA, 5 mM mercaptoethanol, pH 9.0, treat the suspension with ultrasound (Hielscher sonicator UP200S, 16 min, amplitude 70%, cycle 0.5);

2) Centrifuge the lysate at 11000 rpm for 20 min, at +4° C.;

3) Prepare sucrose gradient (20-60%) in 35 ml tubes, in buffer containing 50 mM Na citrate, 5 mM Na borate, 2 mM EDTA, 0.5% TX-100;

4) Overlay 5 ml of the VLP sample over the sucrose gradient;

5) Centrifuge 6 h using SW32 rotor, Beckman (25000 rpm, at +18° C.).

6) Divide the content of each gradient tube in 6 ml fractions. Pool corresponding fractions;

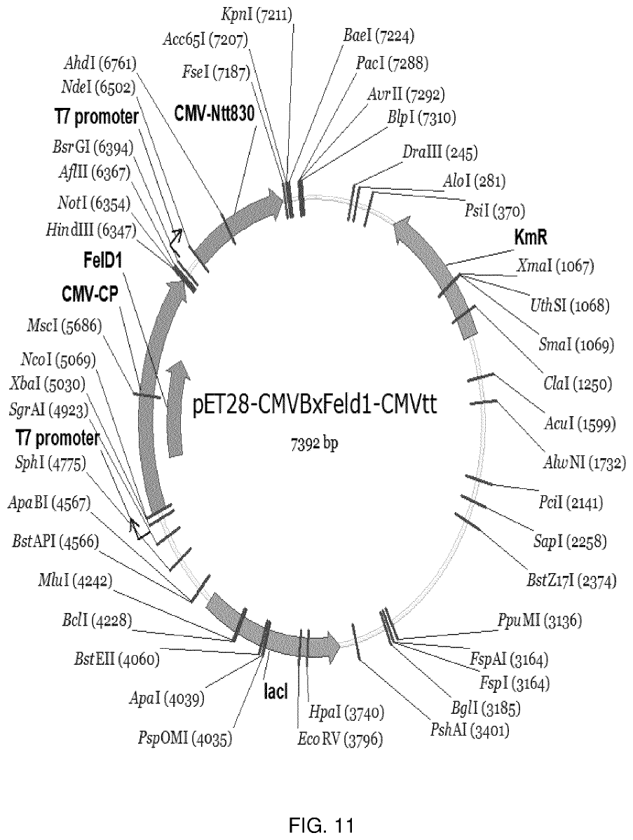

7) Analyse gradient fractions on SDS-PAGE (FIG. 12A);

8) SDS-PAGE analysis suggest the presence of mosaic VLPs in 2nd sucrose gradient fraction. Dilute 24 ml of 3rd fraction with 24 ml of buffer (5 mM Na borate, 2 mM EDTA, pH 9.0);

9) Collect the VLPs by ultracentrifugation using rotor Type 70 (Beckman Optima, L100XP ultracentrifuge; 4 h, at 50 000 rpm, 5° C.);

10) Solubilize the pellet in 3 ml of 5 mM Na borate, 2 mM EDTA, pH 9.0;

11) Overly the VLP suspension on the top of 20% sucrose “cushion” (in 5 mM Na borate, 2 mM EDTA, pH 9.0 buffer);

12) Collect the VLPs by ultracentrifugation using rotor TLA100.3 (Beckman; 1 h, at 72000 rpm, 5° C.);

13) Solubilize the pellet in 2 ml of 5 mM Na borate, 2 mM EDTA, pH 9.0, ON, 4° C.;

14) Analyse the VLPs after purification on SDS-PAGE gel (FIG. 12B) as well as under EM (FIG. 12C).

### Example 10

### Immunization of Mice with Mosaic Particle CMV-M-Fel

Groups of four female Balb/c mice were either immunized with CMV-M-Fel or Fel d1 chemically coupled to CMVNtt830 VLP or recombinant Fel d1 extract. VLPs were formulated in 150 mM PBS, pH 7.4 and 25 ug were injected s.c. in a volume of 150 ul. Recombinant Fel d1 extract was formulated in PBS and 10 ug were injected s.c. Two weeks later, mice were bled and antibody levels against Fel d1 were determined by ELISA. All immune sera were tested by ELISA against recombinant Fel d1 as described by Schmitz N, et al., J Exp Med (2009) 206:1941-1955.

ELISA:

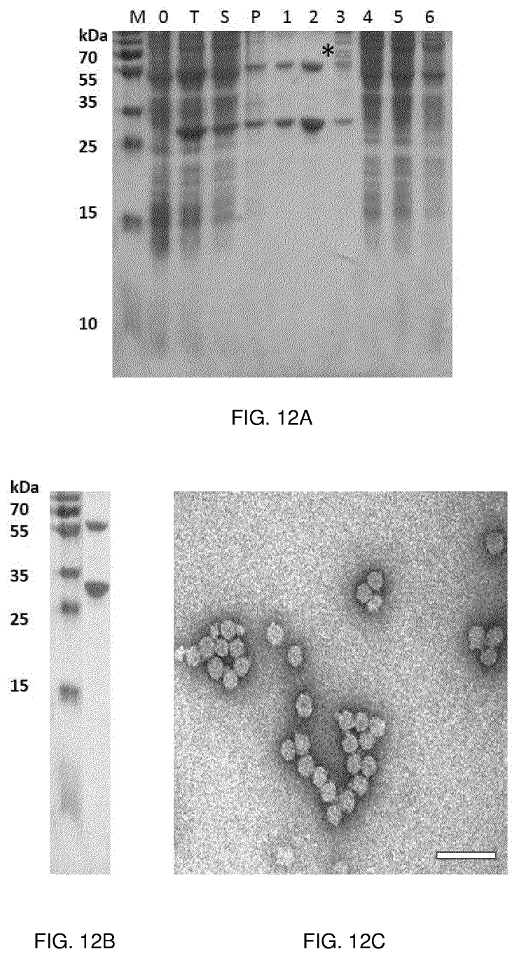

The antibody response in mouse sera were analyzed at the indicated time. For determination of Fel d1 specific antibody titers, ELISA plates (Nunc Immuno MaxiSorp, Rochester, N.Y.) were coated overnight at 4° C. with 100 μl of Fel d1 (1 μg/ml) in PBS. In order to avoid unspecific binding the ELISA plates were blocked with 200 μl of 2% BSA in PBST and incubated for 2 hours at RT. The serum samples were diluted in 2% BSA/PBST. Pre-diluted sera were transferred onto the coated plates and further serial diluted to obtain antibody titers based on OD50 calculation. After 2 hours of incubation at RT, the ELISA plates were washed 5× with 200 μl of PBST. Binding of serum antibodies was detected by horse-radish peroxidase-conjugated goat anti-mouse IgG (Jackson ImmunoResearch). The detection antibody was diluted 1:1000 in 2% BSA/PBST and a volume of 100 μl per sample was transferred. The plates were incubated for 1 hour at RT. ELISA plates were washed as described before. Prior washing the substrate solution was prepared. To this end, 1 tablet (10 mg) of OPD (1,2-Phenylenediamine dihydrochloride) and 9 μl of 30% H₂O₂ was dissolved in 25 ml citric acid buffer (0.066 M Na₂HPO₄, 0.035 M citric acid, pH 5.0). A volume of 100 μl of the substrate solution was pipetted onto the plates and exactly incubated for 7 minutes at RT. To stop the reaction 50 μl of stop solution (5% H₂SO₄ in H₂O) was directly pipetted onto the plates. Absorbance readings at 450 nm of the 1, 2-Phenylenediamine dihydrochloride color reaction were analyzed. FIG. 13 shows antibodies specific for Fel d1 (FIG. 13).

### Example 11

### Immunization with CMV-M-Fel Protects Against Anaphylactic Reaction

Mice were rendered allergic to Fel d1 by sensitizing i.p. with 1 ug natural Fel d1 isolated from cat fur formulated in Alum on day 1. Mice were immunized then with CMV-M-Fel or CMV-Ntt830 VLP (30 ug in PBS, day 14) before i.v. systemic anaphylactic reactions when by temperature drop was determined.

Sensitization

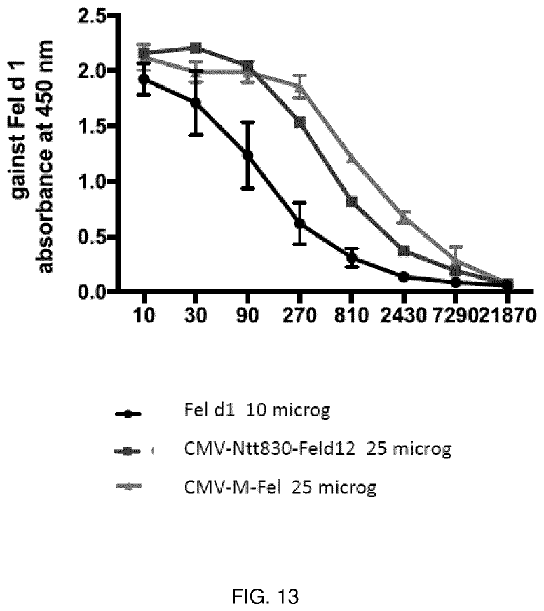

Mice were sensitized to Fel d1 by i.p. injection of natural Fel d1 extract (1 ug) (Indoor Biotechnologies) formulated in 200 μl Alum on day 0. Mice were then vaccinated with CMV-M-Fel or CMV-Ntt830 VLP for the control group (30 ug in 200 ul PBS at day 21). FIG. 14A shows the experimental design to investigate the protective effect of vaccination with CMV-M-Fel against allergic systemic and local reaction.

Systemic Challenge

Challenge was performed i.v with Fel d1 extract in sensitized and vaccinated BALB/c mice. Systemic allergic reactions were determined by temperature drop (FIG. 14B). Graphs are representative for 2 independent experiments. Mean values+/−SEM of 5 mice per group are shown. Anaphylaxis curves were analyzed by two-way-Anova test.

### Example 12

### Construction and Expression of a Mosaic Particle Containing Modified Coat Protein of CMV and In-Fused Internal Repeats of the Circumsporozoite Protein (CSP) of P. falciparum (CMV-M-CSP)

For cloning of Plasmodium falciparum CS protein fragment, namely 19 repeats of NANP (SEQ ID NO:43) leading to SEQ ID NO:44 (19nanp) from central repeat region, the sequence of SEQ ID NO:45 was obtained from commercial source (gene synthesis):

The sequence SEQ ID NO:45 codes also for 3′terminal part of CMV-Ntt830 gene and necessary restriction sites BamHI and HindIII.

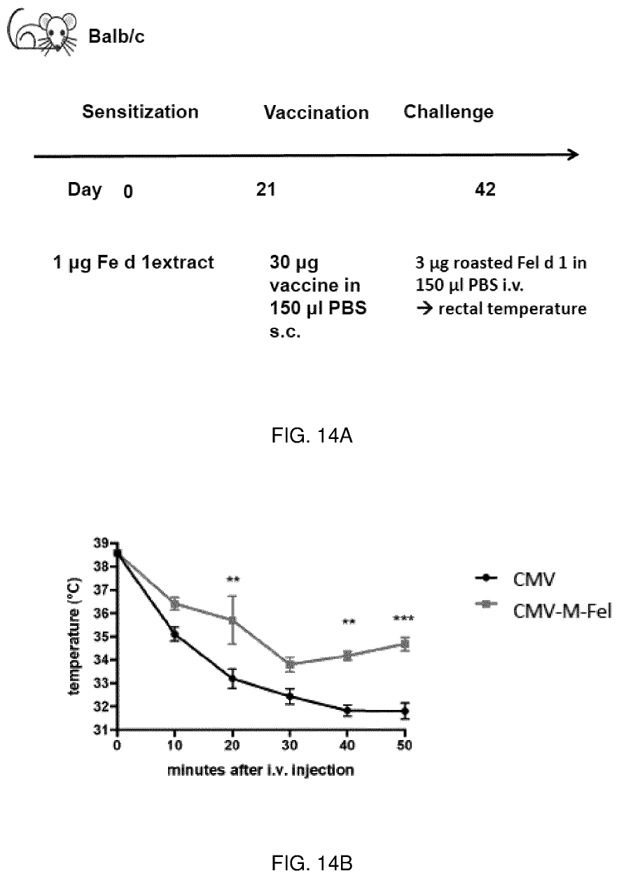

Next, DNA fragment from gene synthesis product was treated with BamHI/HindIII and subloned into helper vector pTZ-CMVB2 (see Example 5). Correct clones, containing 19NANP insert was found using restriction site analysis (BamHI/HindIII). Plasmid clones with correct restriction enzyme pattern were resequenced. As a next step, CMVB-19NANP fragment after NcoI/HindIII treatment was subcloned into helper vector pETDu-CMV-Ntt830 (see Example 5). To construct the expression vector without Amp resistance gene, the whole cassette containing CMV-19NANP fusion and unmodified CMV-Ntt830 gene from pETDu-CMV-Ntt830 was excised and subcloned into NcoI/BlpI site of pET28a+(Novagen), resulting in expression vector pET28-CMVB2x19nanp-CMVtt. Plasmid map and sequence details are shown in FIG. 15.

The amino acid sequence of this preferred chimeric CMV polypeptide in accordance with the present invention referred to as “CMV-Ntt830-19NANP” (or “CMV-Ntt830-19nanp”, as interchange-ably used herein) is SEQ ID NO:46. This amino acid sequence of this preferred chimeric CMV polypeptide comprise glycine-serine linkers flanking the introduced 19nanp protein of SEQ ID NO:44 at both termini, namely a 15 amino acid long GS-linker (SEQ ID NO:30) directly at the N-terminus of the introduced 19nanp protein and a 12 amino acid long GS-linker (SEQ ID NO:47) at the C-terminus of the introduced 19nanp protein. The corresponding nucleotide sequence of this preferred chimeric CMV polypeptide CMV-Ntt830-19NANP is described in SEQ ID NO:48.

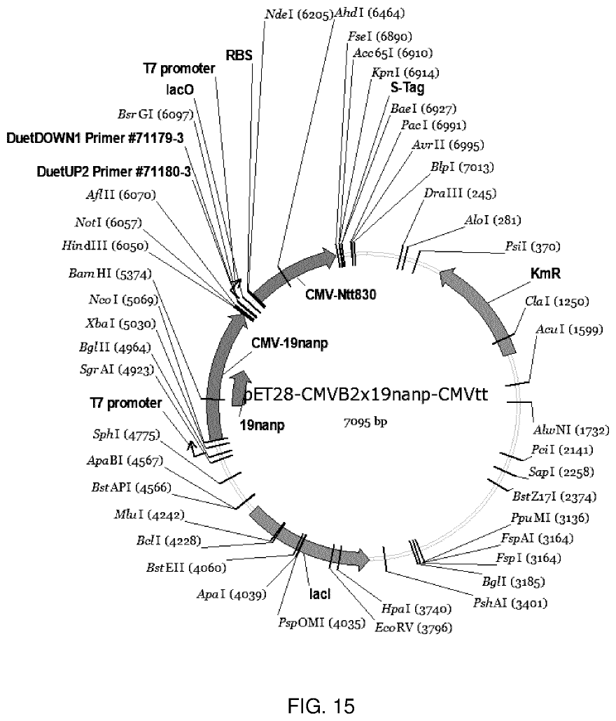

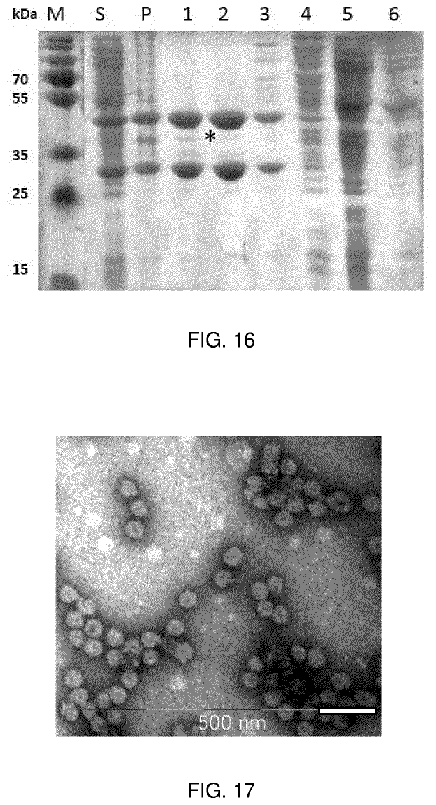

Further, resulting plasmid clone pET28-CMVB2x19nanp-CMVtt for synthesis of mosaic VLPs comprising CMV-Ntt830-19NANP and unmodified CMV-Ntt830 proteins, i.e. CMV-M-CSP, was used for transformation of E. coli C2566 cells. For isolation of CMV-M-CSP, E. coli cell cultivation, treatment of biomass and purification was carried out as described in Example 6. Analysis of the VLPs after sucrose gradient purification in SDS-PAGE gels is shown in FIG. 16. Images of purified CMV-M-CSP after electron microscopy analysis are shown in FIG. 17.

### Example 13

### Protective Efficacy of CMV-M-CSP

Groups of four female Balb/c mice are immunized with CMV-M-CSP. VLPs are formulated in 150 mM PBS, pH 7.4 and 10 ug injected s.c. in a volume of 150 ul. In order to check the vaccine efficacy, 6 female BALB/c inbred mice per group and 8 female CD1 outbred mice per group, 8 weeks old, are purchased from Harlan, United Kingdom, and vaccinated intramuscularly (i.m) with 20 μg (50 μL) per mouse of CMV-Ntt830 or CMV-M-CSP. The vaccinations are done at day 0 and day 21. The samples (blood) were collected before each vaccination on days 0, 21, 42 and CSP specific immune responses are measured by ELISA. On Day 42, the mice are infected with P. berghei replacement expressing CSP protein of P. falciparum protein. The parasitaemia is checked daily beginning on fourth day after the challenge until the mice reach 1% parasitaemia.

### Measurement of CSP-Specific Antibody Responses by ELISA

For assessment of antibody production, total IgG and its subclasses, Enzyme-linked immunosorbent assays (ELISAs) are performed. For that, 96-well microtitre ELISA plates (Thermo Scientific, Nottingham, UK) are coated with 100 μL per well in a concentration of 1 μg/mL of purified CSP and diluted in carbonate buffer (CBB) 50 mM at pH=9.6. and incubated overnight at 4° C. In the next day the plates are filled with 200 μL of 2% BSA-PBS to avoid nonspecific binding and incubated at room temperature for 2 h. Sera from immunized mice are then diluted in 0.2% BSA-PBS buffer, initially starting with 1 in 100 and followed by eleven 1/3 serial dilution in the ELISA plates. For the total IgG measurement, 100 μL per well of goat anti-mouse IgG diluted to 1:2000 (Secondary Antibody, HRP conjugate (ThermoFisher, Paisley, UK) are added and incubated for 1 h at room temperature. For assessment of IgG subclass, goat anti-mouse IgG subclass (goat anti-mouse IgG1, IgG2a, IgG2b HRP coupled, Life Technologies) are used in a dilution of 1:2000, and incubated for 1 h at room temperature. To develop the reaction, 100 μL/well of TMB substrate (Sigma-Aldrich) is applied and incubated at RT for 10 min and under aluminium foil for light protection. After that, the reaction is stopped with 0.5 M H2SO4 (100 μL/well) and the plates read using microplate reader at 450 nm. The titres are expressed as dilutions leading to half-maximal OD (OD50).

### Measurement of Protective Efficacy

The parasite used in this study is the P. bergei which express the CSP protein of P. falciparum (Sci Rep. 2015 Jul. 3; 5:11820. doi: 10.1038/srep11820.). Female Anopheles stephensi mosquitoes are fed with infected Tuck-ordinary (TO) mice. The infected mosquitoes are kept for 21 days in a humidified incubator at a temperature of 19 to 21° C. on a 12-h day-night cycle and fed with a fructose-p-aminobenzoic acid (PABA) solution. After 21 days, salivary glands are dissected from mosquitoes into Schneider's media (Pan Biotech, Aidenbach, Germany) and sporozoites gently liberated using a glass homogenizer. The sporozoites are diluted for a concentration of 1000 parasites in 100 μL and intravenously injected into the tail vein of the mice. Parasitemea. The parasitaemia is checked daily beginning on fourth day after the challenge until the mice reach 1% parasitaemia.

### Example 14

### Construction and Expression of VLPs Containing CMV Fusions with Alpha-Synuclein Peptides

CMV-alpha-synuclein-chimeric CMV polypeptides in accordance with the present invention have been prepared comprising the alpha-synuclein peptides egy (SEQ ID NO:49), kne (SEQ ID NO:50), and mdv (SEQ ID NO:51).

These alpha-synuclein peptides have been inserted between amino acid residues Ser(88) and Tyr(89) of CMV-Ntt830 (SEQ ID NO:5). The amino acid sequences of these preferred chimeric CMV polypeptides in accordance with the present invention as follows:

Amino acid sequence of “CMV-Ntt830-egy”: SEQ ID NO:52;

Amino acid sequence of “CMV-Ntt830-kne”: SEQ ID NO:53;

Amino acid sequence of “CMV-Ntt830-mdv”: SEQ ID NO:54;

The amino acid sequences of these preferred chimeric CMV polypeptides further comprise glycine-serine linkers flanking the introduced alpha-synuclein peptides at both termini. All preferred fusions proteins of SEQ ID NO:52 to SEQ ID NO:54 comprise a GGGS-linker (SEQ ID NO:10) directly at the N-terminus of the introduced alpha-synuclein peptide and a GGGSGS-linker (SEQ ID NO:11) at the C-terminus of the introduced alpha-synuclein peptide.

The corresponding nucleotide sequences of said preferred chimeric CMV polypeptides are as follows:

Nucleic acid sequence of “CMV-Ntt830-egy”: SEQ ID NO:55;

Nucleic acid sequence of “CMV-Ntt830-kne”: SEQ ID NO:56;

Nucleic acid sequence of “CMV-Ntt830-mdv”: SEQ ID NO:57;

For the introduction of DNA coding for alpha-synuclein peptide variants in expression vector, following oligonucleotides were used in PCR reactions whereas the template in all PCRs was pET-CMV-Ntt830:

1^{st} PCR Forward: CM-egyF (SEQ ID NO:58)

Reverse: CMcpR (SEQ ID NO:59)

2^{nd} PCR Forward: CM-kneF (SEQ ID NO:60)

Reverse: CMcpR (SEQ ID NO:59)

3^{rd} PCR Forward: CM-mdvF (SEQ ID NO:61)

Reverse: CMcpR (SEQ ID NO:59)

All PCR fragments were directly ligated into pTZ57R/T vector, and corresponding insert-containing plasmid clones were isolated after transformation in E. coli XL1 cells. Several plasmid clones containing DNA of corresponding PCR product were sequenced using a BigDye cycle sequencing kit and an ABI Prism 3100 Genetic Analyser (Applied Biosystems). After sequencing, the alpha-synuclein fragment-containing CMV 3′ end fragments were excised from pTZ vector and ligated into pET-CMV-Ntt830B helper vector (see Example 1), using BamHI and HindIII restriction enzyme sites. The correct clones were selected after BamHI/HindIII restriction endonuclease tests.

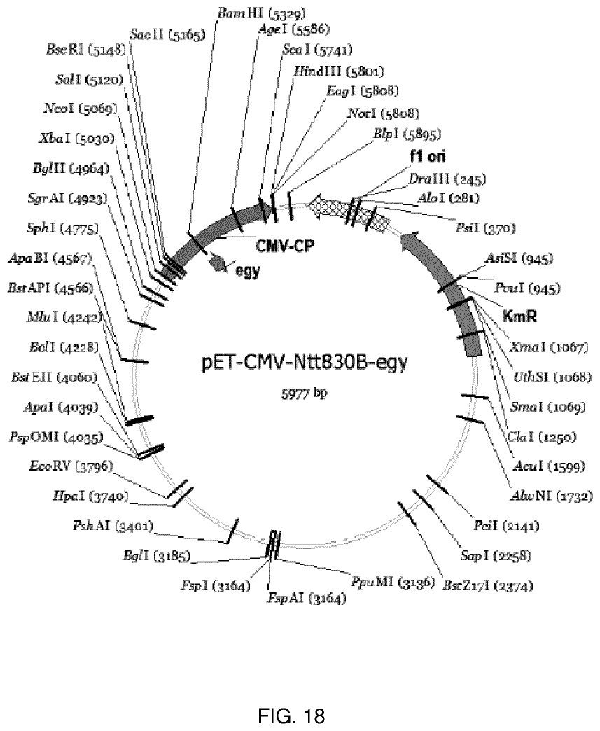

Further, plasmid clones pET-CMV-Ntt830B-egy, pET-CMV-Ntt830B-kne and pET-CMV-Ntt830B-mdv were used for transformation of E. coli C2566 cells. Plasmid map and sequence details are shown in FIG. 18.

For isolation of corresponding alpha-synuclein peptide-containing VLPs, E. coli cell cultivation, treatment of biomass and purification was effected as described in Example 2.

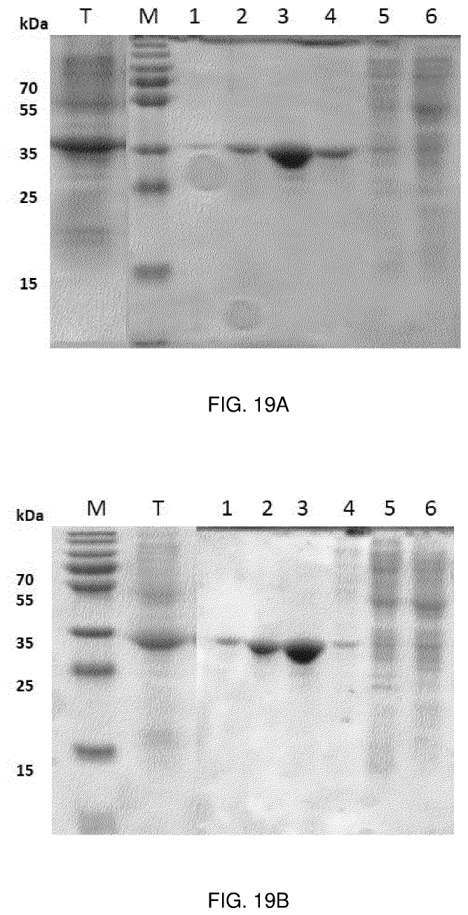

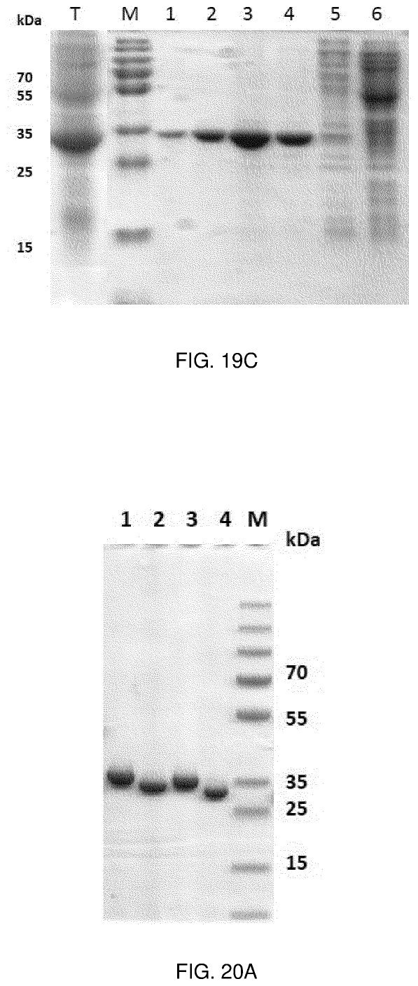

Analysis of the VLPs after sucrose gradient purification in SDS-PAGE gels is shown in FIG. 19A, FIG. 19B and FIG. 19C as well as FIG. 20A. Electromicroscopy images are shown in FIG. 20B, FIG. 20C and FIG. 20D.

### Example 15

### Construction and Expression of a Mosaic Particle Containing Modified Coat Protein of CMV and In-Fused Feline Interleukin 5 (CMV-M-Fel-IL-5)

For cloning of a modified coat protein of CMV comprising feline IL-5 antigen, two different vectors was constructed.

A first vector allowing the introduction of amino acid linkers comprising at least one Gly, at least one Ser, and at least Glu, and even further comprising at least one Asp, on both sides of the fel IL-5 antigen was constructed, using PCR mutagenesis and oligonucleotides as follows:

1^{st} PCR reaction:

Forward: 830-NcoF (SEQ ID NO:33)

Reverse: Cmded-BamR (SEQ ID NO: 121)

Template: pETDu-CMVB2xArah202-CMV-tt

2^{nd} PCR reaction:

Forward: CMded-BamF (SEQ ID NO: 122)

Reverse: CM-cpR (SEQ ID NO:26)

Template: pETDu-CMVB2xArah202-CMV-tt

After amplification of the gene fragments, the corresponding PCR products were directly cloned into the pTZ57R/T vector (InsTAclone PCR Cloning Kit, Fermentas #1(1214). E. coli XL1-Blue cells were used as a host for cloning and plasmid amplification. To avoid RT-PCR errors, several CMV-Ntt830 gene-containing pTZ57 plasmid clones were sequenced using a BigDye cycle sequencing kit and an ABI Prism 3100 Genetic analyzer (Applied Biosystems). After sequencing, pTZ-plasmid clone containing product from the 1^{St} PCR reaction was cut with NcoI/BamHI restrictases, the clone from 2^{nd} PCR was cut with BamHI/HindIII and joined in ligation reaction with the helper vector pETDu-CMVB2xArah202-CMV-tt, which was cut with NcoI/HindIII. Here we used the plasmid pETDu-CMVB2xArah202-CMV-tt as a helper vector to generate new CMV-based expression vectors. The NcoI/HindIII treatment completely removed the CMVB2xArah202 gene. The ligation of three DNA fragments resulted in helper plasmid pETDu-CMVB3d-CMVtt. The vector contains CMV-Ntt830 gene with the Th cell epitope derived from tetanus toxin in both encoded proteins, and introduced sequences coding for amino acid linkers comprising at least one Gly, at least one Ser, and at least Glu, and even further comprising at least one Asp, in detail comprising Gly-Ser linkers with additional Asp-Glu-Asp stretches and BamHI/SpeI sites for subcloning of antigen DNA sequences in the CMV-Ntt830 gene under the first T7 promotor.

Moreover, a second vector allowing the introduction of a GS-linker on the N terminus of the fel IL-5 antigen and an amino acid linker comprising at least one Gly, at least one Ser, and at least Thr on the C terminus of the fel IL-5 antigen was constructed, using PCR mutagenesis and oligonucleotides as follows:

3^{rd} PCR reaction:

Forward: CM-BamSpeF (SEQ ID NO: 137)

Reverse: CM-cpR (SEQ ID NO:26)

Template: pETDu-CMVB2xArah202-CMV-tt

The PCR product from the 3^{rd} reaction was also directly cloned into the pTZ57R/T vector; resulting ligation mixture was used for the transformation of E. coli XL1-Blue cells. After isolation of plasmid DNA, several clones were sequenced. Correct plasmid clone was cut with BamHI/HindIII restrictases and resulting fragment subcloned into pETDu-CMVB2xArah202-CMV-tt cut with the same enzymes. The ligation reaction resulted in the helper plasmid pETDu-CMVB3-CMVtt.

Feline interleukin 5 (IL5) gene was obtained from gene synthesis service (General Biosystems, USA) in the form of plasmid pET42-felIL5N. For cloning, felIL5 gene was amplified in PCR reaction using following oligonucleotides:

Forward: I5-BamF (SEQ ID NO: 123)

Reverse: I5-SpeR (SEQ ID NO: 124)

Template: pET42-felIL5N

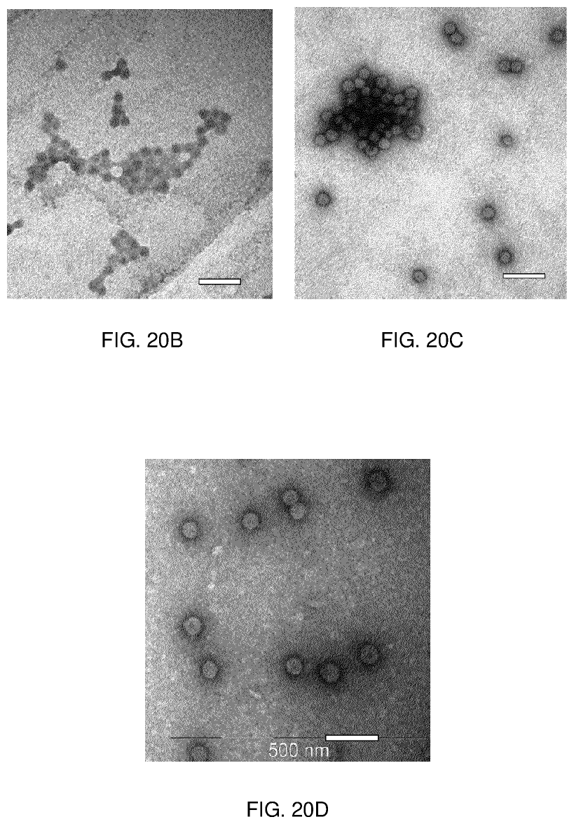

The PCR product was directly ligated into pTZ57 and after plasmid DNA isolation, several clones were sequenced. After identification of the clone without sequence errors the felIL5 gene was further subcloned into pETDu-CMVB3d-CMV-tt or pETDu-CMVB3-CMV-tt in sites BamHI/SpeI. The plasmid maps of the resulting pETDu-CMVB3d-flIL5-CMV-tt and pETDu-CMVB3-flIL5-CMV-tt are shown in FIG. 21A and FIG. 21B. Said plasmid and expression vectors ensure and serves for expression of mosaic VLPs comprising CMV-Ntt830-fel-IL-5 and unmodified CMV-Ntt830 proteins, i.e. CMV-M-fel-IL-5.

CMV-Ntt830-fel-IL-5 comprises the feline IL-5 protein of SEQ ID NO:125 which is flanked by amino acid linkers comprising at least one Gly, at least one Ser and at least one Glu. In detail, said feline IL-5 protein of SEQ ID NO:125 is directly flanked at its N-terminus by the 18 amino acid long GSED-linker of SEQ ID NO:126 and directly flanked at its C-terminus by the 15 amino acid long GSED-linker of SEQ ID NO:127. Further, said aforementioned described entire construct, i.e. the construct of SEQ ID NO:125 flanked by the described GSED-linkers, is inserted between positions corresponding to Ser(88) and Tyr(89) of SEQ ID NO:5 of CMV-Ntt830 leading to CMV-Ntt830-fel-IL-5. The amino acid sequence of this preferred chimeric CMV polypeptide in accordance with the present invention referred to as “CMV-Ntt830-fel-IL-5” is SEQ ID NO:128. The corresponding nucleotide sequence of this preferred chimeric CMV polypeptide CMV-Ntt830-fel-IL-5 is described in SEQ ID NO:129.

CMV-Ntt830-fel-IL-5* comprises the feline IL-5 protein of SEQ ID NO:125 which is flanked by a GS-linker on the N terminus and an amino acid linker comprising at least one Gly, at least one Ser, and at least Thr on the C terminus of the fel IL-5 antigen. In detail, said feline IL-5 protein of SEQ ID NO:125 is directly flanked at its N-terminus by the 15 amino acid long GS-linker of SEQ ID NO:30 and directly flanked at its C-terminus by the 11 amino acid long GST-linker of SEQ ID NO:138. Further, said aforementioned described entire construct, i.e. the construct of SEQ ID NO:125 flanked by the described GS and GST-linkers, is inserted between positions corresponding to Ser(88) and Tyr(89) of SEQ ID NO:5 of CMV-Ntt830 leading to CMV-Ntt830-fel-IL-5*. The amino acid sequence of this chimeric CMV polypeptide in accordance with the present invention referred to as CMV-Ntt830-fel-IL-5* is SEQ ID NO:139. The corresponding nucleotide sequence of this chimeric CMV polypeptide CMV-Ntt830-fel-IL-5* is described in SEQ ID NO:140.

Thus, for expression and purification of mosaic CMV-M-fel-IL-S and CMV-M-fel-IL-5*, E. coli C2566 (New England Biolabs, USA) competent cells were transformed with the plasmid pETDu-CMVB3d-flIL5-CMVtt and pETDu-CMVB3-flIL5-CMVtt.

After selection of clones with the highest expression level of target proteins, E. coli cultures were grown in 2TY (Trypton 1.6%, yeast extract 1%, 0.5% NaCl, 0.1% glucose) medium containing ampicillin (100 mg/1) on a rotary shaker at 30° C. to the OD(600) value of 0.8-1.0. Then, the cells were induced with 0.2 mM IPTG, and the medium was supplemented with 5 mM MgCl₂. Incubation was continued on the rotary shaker at 20° C. for 18 h. The resulting biomass was collected by low-speed centrifugation and was frozen at −20° C. Biomass output—approx. 14 g wet biomass/1 culture, OD(600) was 7.6 at the end of cultivation.

The purification of mosaic VLPs comprising CMV-Ntt830-fel-IL-5 or CMV-Ntt830-fel-IL-5* and unmodified CMV-Ntt830 proteins (said mosaic VLPs referred to as CMV-M-fel-IL-5 and CMV-M-fel-IL-5*) includes the following steps:

1) suspend 1.5 g biomass in 10 ml of 20 mM Tris-HCl (pH 8), 5 mM EDTA, 5 mM mercaptoethanol, 5% glycerol, 10% sucrose, treat the suspension with ultrasound (Hielscher sonicator UP200S, 16 min, amplitude 70%, cycle 0.5);

2) Centrifuge the lysate at 11000 rpm for 20 min, at +4° C.;

3) Prepare sucrose gradient (20-60%) in 35 ml tubes, in buffer containing 20 mM Tris-HCl (pH 8), 5 mM EDTA, 5 mM mercaptoethanol, 5% glycerol, 0.5% TX-100;

4) Overlay 5 ml of the VLP sample over the sucrose gradient. Prepare 2 tubes;

5) Centrifuge 6 h using SW32 rotor, Beckman (25000 rpm, at +18° C.).

6) Divide the content of each gradient tube in 6 ml fractions. Pool corresponding fractions;

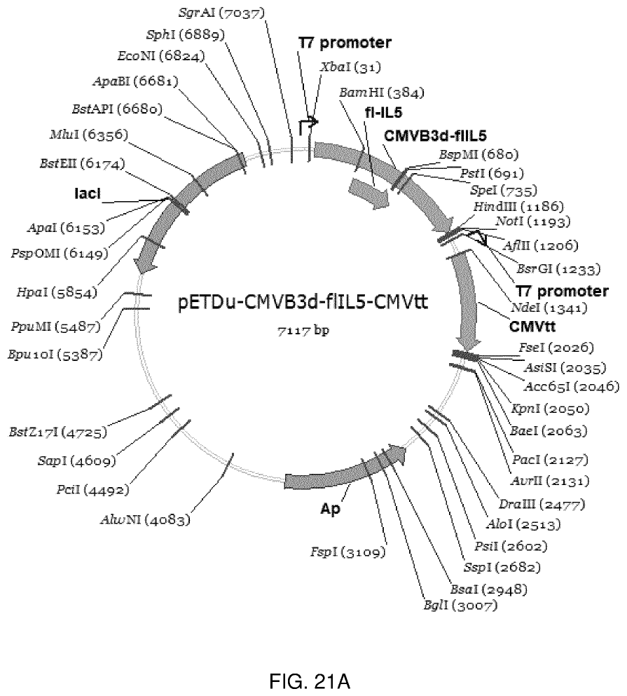

7) Analyze gradient fractions on SDS-PAGE (FIG. 22A and FIG. 22B).

8) SDS-PAGE analysis suggest the presence of mosaic VLPs in 2nd and 3rd sucrose gradient fraction. Dilute 2nd and 3rd fraction with equivalent amount of buffer (20 mM Tris-HCl, 5 mM EDTA, pH 8.0);

9) Collect the VLPs by ultracentrifugation using rotor Type 70 (Beckman Optima, L100XP ultracentrifuge; 4 h, at 50 000 rpm, 5° C.);

10) Solubilize the pellet in 2 ml of 20 mM Tris-HCl (pH 8), 5 mM EDTA, 5 mM mercaptoethanol, 5% glycerol, 10% sucrose;

11) Overly the VLP suspension on the top of 20% sucrose “cushion” (in 20 mM Tris-HCl (pH 8), 5 mM EDTA, 5 mM mercaptoethanol, 5% glycerol buffer);

12) Collect the VLPs by ultracentrifugation using rotor TLA100.3 (Beckman; 1 h, at 72000 rpm, 5° C.);

13) Solubilize the pellet in 2 ml of 20 mM Tris-HCl (pH 8), 5 mM EDTA, 5 mM mercaptoethanol, 5% glycerol, 10% sucrose, ON, 4° C.;

14) Clarify the suspension by centrifugation (5 min, 13000 rpm, Eppendorf 5418)

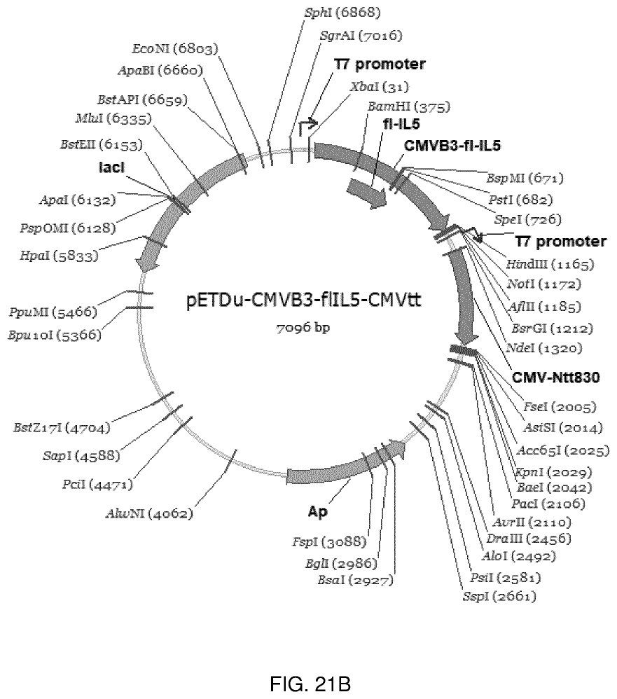

15) Analyze the VLPs after purification on SDS-PAGE gel (FIG. 23A and FIG. 23C) as well as under EM (FIG. 23B and FIG. 23D).

The EM pictures show that the mosaic VLPs comprising CMV-Ntt830-fel-IL-5 and unmodified CMV-Ntt830 proteins and thus the mosaic VLPs CMV-M-fel-IL-5 contains a significant less number of aggregated VLPs as compared to the mosaic VLPs comprising CMV-Ntt830-fel-IL-5* and unmodified CMV-Ntt830 proteins and tus the mosaic VLPs CMV-M-fel-IL-5*. This finding was confirmed by dynamic light scattering (DLS) analysis of both of said mosaic VLPs in accordance with the present invention.

### Example 16

### Construction and Expression of a Mosaic Particle Containing Modified Coat Protein of CMV and In-Fused Duplicated Feline Interleukin 5 (CMV-M-2Xfel-IL-5)

For cloning of a modified coat protein of CMV comprising two copies of feline IL-5 antigen, a corresponding vector allowing the introduction of amino acid linkers comprising at least one Gly, at least one Ser, and at least Glu, and even further comprising at least one Asp, on both sides of the fel IL-5 antigens and an amino acid linker to connect both feline I1-5 antigens was constructed, using PCR mutagenesis and oligonucleotides as follows:

1^{st} PCR reaction:

Forward: I5-BamF (SEQ ID NO:123)

Reverse: 2xIL5-gsKpnR (SEQ ID NO: 130)

Template: pETDu-CMVB3d-flIL5-CMV-tt

2^{nd} PCR reaction:

Forward: 2xIL5-gsKpnF (SEQ ID NO: 131)

Reverse: I5-SpeR (SEQ ID NO:124)

Template: pETDu-CMVB3d-flIL5-CMV-tt

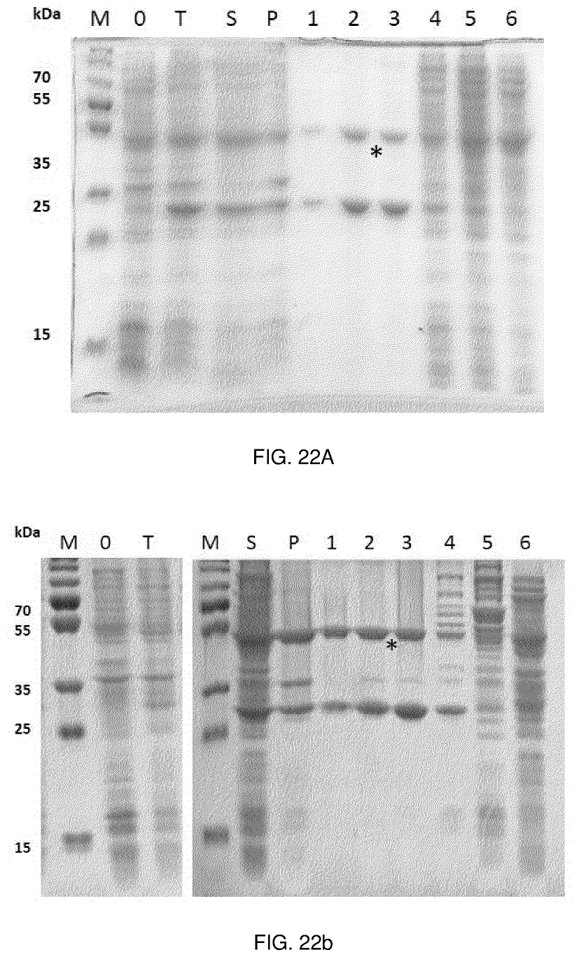

After amplification of the gene fragments, the corresponding PCR products were directly cloned into the pTZ57R/T vector (InsTAclone PCR Cloning Kit, Fermentas #1(1214). E. coli XL1-Blue cells were used as a host for cloning and plasmid amplification. To find plasmids without PCR errors, several fel-IL5 gene-containing pTZ57 plasmid clones were sequenced using a BigDye cycle sequencing kit and an ABI Prism 3100 Genetic analyzer (Applied Biosystems). After sequencing, correct pTZ-plasmid clone containing product from 2^{nd} PCR reaction was cut with Kpn2/EcoRI restrictases, and the resulting fragment was ligated into pTZ plasmid containing the PCR product from 1^{st} PCR reaction cut with the same restrictases. The ligation reaction resulted in helper plasmid pTZ-2xflIL5, containing two copies of flIL5 gene connected with a Gly-Ser linker. The plasmid containing duplicated felIL5 was purified from XL1 cells. The 2xfel-IL5 gene was further excised using BamHI/SpeI restrictases and ligated into expression vector pETDu-CMVB3d-CMVtt in the same restriction sites. The resulting vector contains CMV-Ntt830 with introduced sequence coding for two feline IL5 genes separated by a Gly-Ser linker and flanked by amino acid linkers comprising at least one Gly, at least one Ser, and at least Glu, and even further comprising at least one Asp, in detail comprising Gly-Ser linkers with additional Asp-Glu-Asp stretches. The plasmid map of the resulting pETDu-CMVB3d-2xflIL5-CMV-tt is shown in FIG. 24. Said plasmid and expression vector ensures and serves for expression of mosaic VLPs comprising CMV-Ntt830-2xfel-IL-5 and unmodified CMV-Ntt830 proteins, i.e. CMV-M-2xfel-IL-5.

CMV-Ntt830-2xfel-IL-5 comprises two copies of the feline IL-5 protein of SEQ ID NO:125 connected with a Gly-Ser linker (SEQ ID NO:30) and flanked by amino acid linkers comprising at least one Gly, at least one Ser and at least one Glu. In detail, said sequence of two copies of feline IL-5 protein of SEQ ID NO:125 connected with a Gly-Ser linker (SEQ ID NO:30) are directly flanked at its N-terminus by the 18 amino acid long GSED-linker of SEQ ID NO:126 and directly flanked at its C-terminus by the 15 amino acid long GSED-linker of SEQ ID NO:127. Further, said aforementioned described entire construct, i.e. the construct of two copies of SEQ ID NO:125 connected with SEQ ID NO:30 and flanked by the described GSED-linkers, is inserted between positions corresponding to Ser(88) and Tyr(89) of SEQ ID NO:5 of CMV-Ntt830 leading to CMV-Ntt830-2xfel-IL-5.

The amino acid sequence of this preferred chimeric CMV polypeptide in accordance with the present invention referred to as “CMV-Ntt830-2xfel-IL-5” is SEQ ID NO:132. The corresponding nucleotide sequence of this preferred chimeric CMV polypeptide CMV-Ntt830-2xfel-IL-5 is described in SEQ ID NO:133.

Thus, or expression and purification of mosaic CMV-M-2xfel-IL-5, E. coli C2566 (New England Biolabs, USA) competent cells were transformed with the plasmid pETDu-CMVB3d-2xflIL5-CMVtt.

After selection of clones with the highest expression level of target proteins, E. coli cultures were grown in 2TY (Trypton 1.6%, yeast extract 1%, 0.5% NaCl, 0.1% glucose) medium containing ampicillin (100 mg/1) on a rotary shaker at 30° C. to the OD(600) value of 0.8-1.0. Then, the cells were induced with 0.2 mM IPTG, and the medium was supplemented with 5 mM MgCl₂. Incubation was continued on the rotary shaker at 20° C. for 18 h. The resulting biomass was collected by low-speed centrifugation and was frozen at −20° C. Biomass output—approx. 15 g wet biomass/1 culture, OD(600) was 8.0 at the end of cultivation.

The purification of mosaic VLPs comprising CMV-Ntt830-2xfel-I15 and unmodified CMV-Ntt830 proteins (said mosaic VLPs referred to as “CMV-M-2xfel-IL-5”) in accordance with the present invention, includes the following steps:

1) suspend 1.5 g biomass in 10 ml of 20 mM Tris-HCl (pH 8), 5 mM EDTA, 5 mM mercaptoethanol, 5% glycerol, 10% sucrose, treat the suspension with ultrasound (Hielscher sonicator UP200S, 16 min, amplitude 70%, cycle 0.5);

2) Centrifuge the lysate at 11000 rpm for 20 min, at +4° C.;

3) Prepare sucrose gradient (20-60%) in 35 ml tubes, in buffer containing 20 mM Tris-HCl (pH 8), 5 mM EDTA, 5 mM mercaptoethanol, 5% glycerol, 0.5% TX-100;

4) Overlay 5 ml of the VLP sample over the sucrose gradient. Prepare 2 tubes;

5) Centrifuge 6 h using SW32 rotor, Beckman (25000 rpm, at +18° C.).

6) Divide the content of each gradient tube in 6 ml fractions. Pool corresponding fractions;

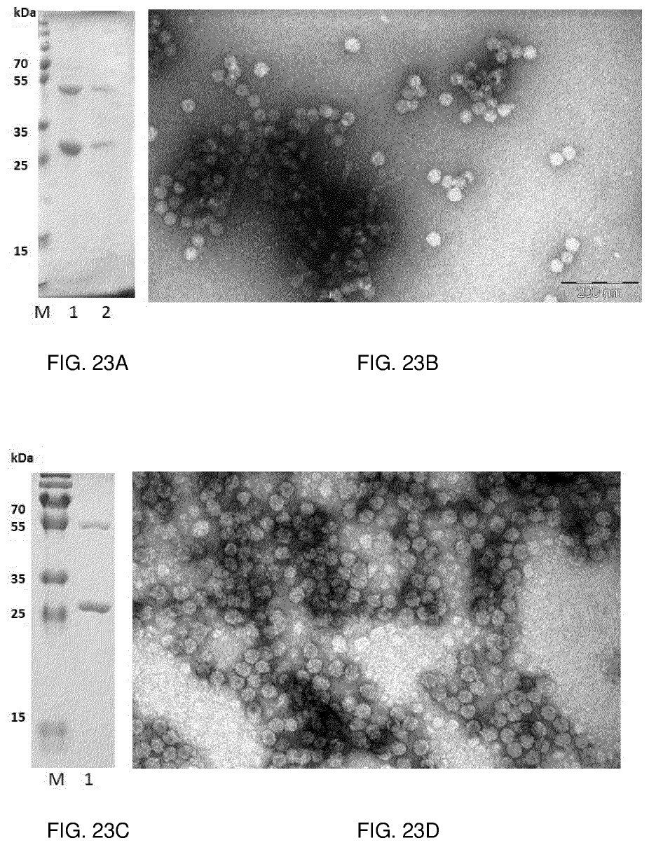

7) Analyze gradient fractions on SDS-PAGE (FIG. 25).

8) SDS-PAGE analysis suggest the presence of mosaic VLPs in 2nd and 3rd sucrose gradient fraction. Pool the 2nd and 3rd fraction, dilute with equivalent amount of buffer (20 mM Tris-HCl, 5 mM EDTA, pH 8.0);

9) Collect the VLPs by ultracentrifugation using rotor Type 70 (Beckman Optima, L100XP ultracentrifuge; 4 h, at 50 000 rpm, 5° C.); 10) Solubilize the pellet in 2 ml of 20 mM Tris-HCl (pH 8), 5 mM EDTA, 5 mM mercaptoethanol, 5% glycerol, 10% sucrose;

11) Overly the VLP suspension on the top of 20% sucrose “cushion” (in 20 mM Tris-HCl (pH 8), 5 mM EDTA, 5 mM mercaptoethanol, 5% glycerol buffer);

12) Collect the VLPs by ultracentrifugation using rotor TLA100.3 (Beckman; 1 h, at 72000 rpm, 5° C.);

13) Solubilize the pellet in 2 ml of 20 mM Tris-HCl (pH 8), 5 mM EDTA, 5 mM mercaptoethanol, 5% glycerol, 10% sucrose, ON, 4° C.;

14) Clarify the suspension by centrifugation (5 min, 13000 rpm, Eppendorf 5418)

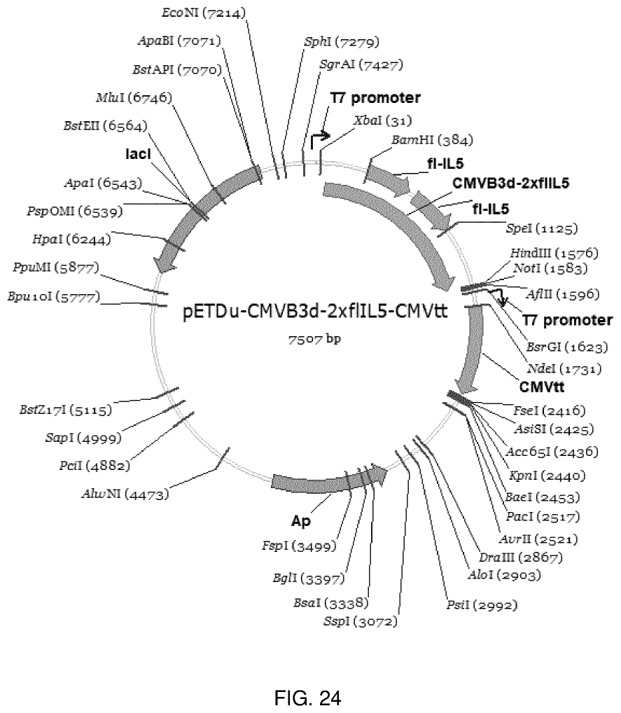

15) Analyse the VLPs after purification on SDS-PAGE gel (FIG. 26A) as well as under EM (FIG. 26B).

### Example 17

Construction and expression of a mosaic particle containing modified Coat Protein of CMV and in-fused canine interleukin 1b (CMV-M-cIL-1b)

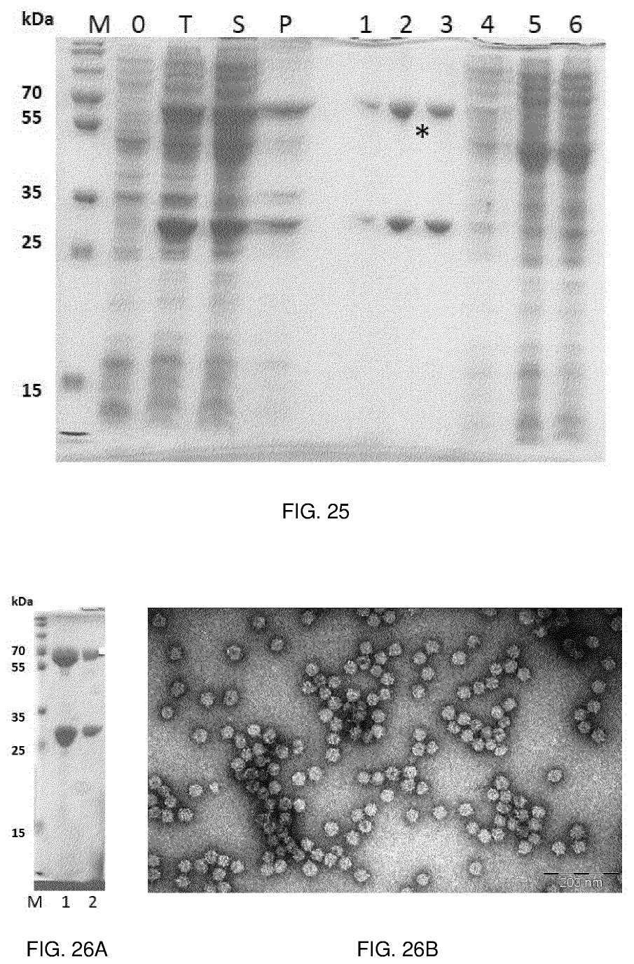

Canine Interleukin 1b gene with flanking BamHI and Spe I sites was obtained from commercial source (gene synthesis product in pUCcIL1b, General Biosystems, USA). The BamHI/SpeI fragment was excised from plasmid pUCcIL1b-BS and ligated into helper vector pETDu-CMVB3d-CMVtt (sites BamHI and SpeI). Resulting plasmid was isolated from E. coli XL1 cells and resequenced to verify the introduced cIL-1b sequence. The plasmid map of the pETDu-CMVB3d-cIL1b-CMV-tt is shown in (FIG. 27). Said plasmid and expression vector ensures and serves for expression of mosaic VLPs comprising CMV-Ntt830-cIL-1b and unmodified CMV-Ntt830 proteins, i.e. CMV-M-cIL-1b.

CMV-Ntt830-cIL-1b comprises the canine IL-1b protein of SEQ ID NO:134 which is flanked by amino acid linkers comprising at least one Gly, at least one Ser and at least one Glu. In detail, said canine IL-1b protein of SEQ ID NO:134 is directly flanked at its N-terminus by the 18 amino acid long GSED-linker of SEQ ID NO:126 and directly flanked at its C-terminus by the 15 amino acid long GSED-linker of SEQ ID NO:127. Further, said aforementioned described entire construct, i.e. the construct of SEQ ID NO:134 flanked by the described GSED-linkers, is inserted between positions corresponding to Ser(88) and Tyr(89) of SEQ ID NO:5 of CMV-Ntt830 leading to CMV-Ntt830-cIL-1b.

The amino acid sequence of this preferred chimeric CMV polypeptide in accordance with the present invention referred to as “CMV-Ntt830-cIL-1b” is SEQ ID NO:135. The corresponding nucleotide sequence of this preferred chimeric CMV polypeptide CMV-Ntt830-cIL-1b is described in SEQ ID NO:136.

Thus, or expression and purification of mosaic CMV-M-cIL-1b, E. coli C2566 (New England Biolabs, USA) competent cells were transformed with the plasmid pETDu-CMVB3d-cIL1b-CMVtt.

After selection of clones with the highest expression level of target proteins, E. coli cultures were grown in 2TY (Trypton 1.6%, yeast extract 1%, 0.5% NaCl, 0.1% glucose) medium containing ampicillin (100 mg/1) on a rotary shaker at 30° C. to the OD(600) value of 0.8-1.0. Then, the cells were induced with 0.2 mM IPTG, and the medium was supplemented with 5 mM MgCl₂. Incubation was continued on the rotary shaker at 20° C. for 18 h. The resulting biomass was collected by low-speed centrifugation and was frozen at −20° C. Biomass output—approx. 15 g wet biomass/1 culture, OD(600) was 8.8 at the end of cultivation.

The purification of mosaic VLPs comprising CMV-Ntt830-cIL-1b and unmodified CMV-Ntt830 proteins (said mosaic VLPs referred to as “CMV-M-cIL-1b”) in accordance with the present invention, includes the following steps:

1) suspend 1.5 g biomass in 10 ml of 20 mM Tris-HCl (pH 8), 5 mM EDTA, 5 mM mercaptoethanol, 5% glycerol, 10% sucrose, treat the suspension with ultrasound (Hielscher sonicator UP200S, 16 min, amplitude 70%, cycle 0.5);

2) Centrifuge the lysate at 11000 rpm for 20 min, at +4° C.;

3) Prepare sucrose gradient (20-60%) in 35 ml tubes, in buffer containing 20 mM Tris-HCl (pH 8), 5 mM EDTA, 5 mM mercaptoethanol, 5% glycerol, 0.5% TX-100;

4) Overlay 5 ml of the VLP sample over the sucrose gradient. Prepare 2 tubes;

5) Centrifuge 6 h using SW32 rotor, Beckman (25000 rpm, at +18° C.).

6) Divide the content of each gradient tube in 6 ml fractions. Pool corresponding fractions;

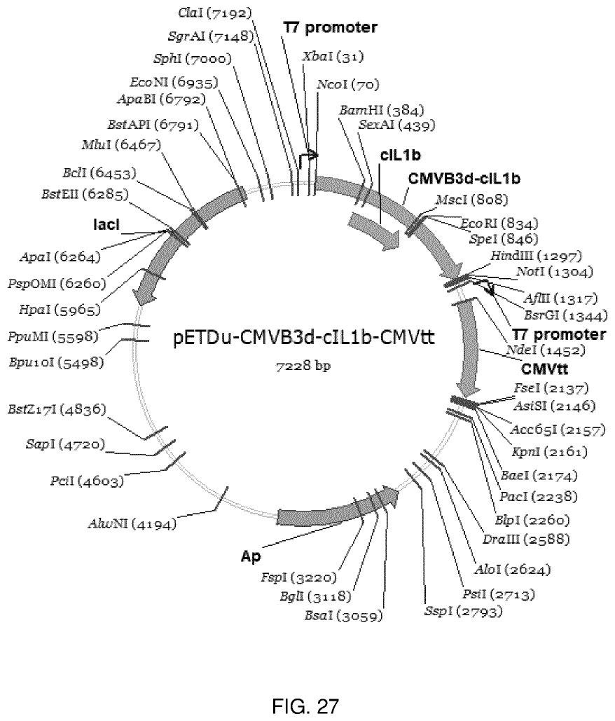

7) Analyze gradient fractions on SDS-PAGE (FIG. 28).

8) SDS-PAGE analysis suggest the presence of mosaic VLPs in 2nd and 3rd sucrose gradient fraction. Pool the 2nd and 3rd fraction, dilute with equivalent amount of buffer (20 mM Tris-HCl, 5 mM EDTA, pH 8.0);

9) Collect the VLPs by ultracentrifugation using rotor Type 70 (Beckman Optima, L100XP ultracentrifuge; 4 h, at 50 000 rpm, 5° C.);

10) Solubilize the pellet in 2 ml of 20 mM Tris-HCl (pH 8), 5 mM EDTA, 5 mM mercaptoethanol, 5% glycerol, 10% sucrose;

11) Overly the VLP suspension on the top of 20% sucrose “cushion” (in 20 mM Tris-HCl (pH 8), 5 mM EDTA, 5 mM mercaptoethanol, 5% glycerol buffer);

12) Collect the VLPs by ultracentrifugation using rotor TLA100.3 (Beckman; 1 h, at 72000 rpm, 5° C.);

13) Solubilize the pellet in 2 ml of 20 mM Tris-HCl (pH 8), 5 mM EDTA, 5 mM mercaptoethanol, 5% glycerol, 10% sucrose, ON, 4° C.;

14) Clarify the suspension by centrifugation (5 min, 13000 rpm, Eppendorf 5418)

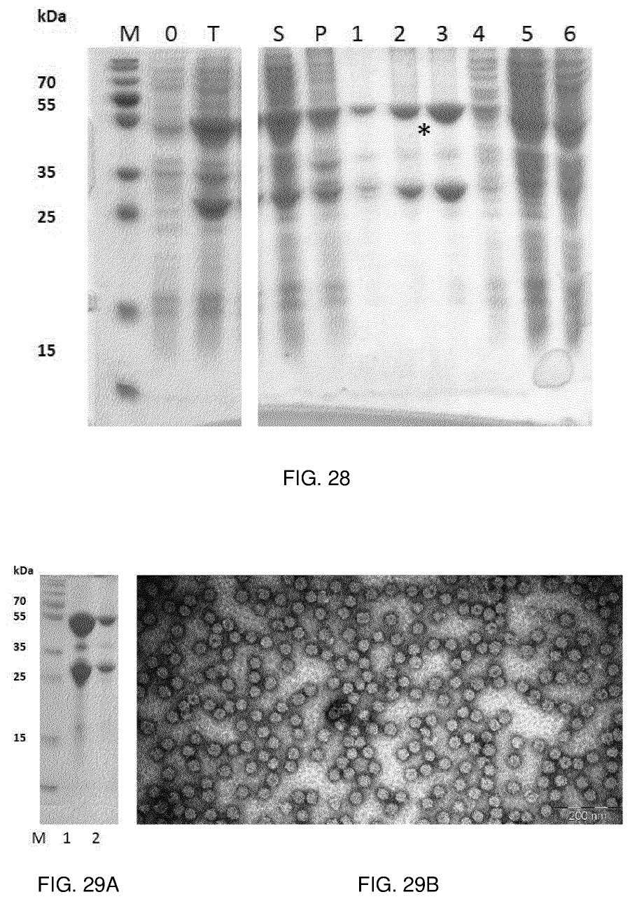

15) Analyze the VLPs after purification on SDS-PAGE gel (FIG. 29A) and under EM (FIG. 29B).

1. A modified virus-like particle (VLP) of cucumber mosaic virus (CMV) comprising at least one fusion protein, wherein said at least one fusion protein comprises
a) a chimeric CMV polypeptide comprising
(i) a CMV polypeptide, wherein said CMV polypeptide comprises a coat protein of CMV or an amino acid sequence having a sequence identity of at least 75% with SEQ ID NO:62; and
(ii) an antigenic polypeptide, wherein said antigenic polypeptide is inserted into said CMV polypeptide,
wherein said insertion of said antigenic polypeptide is between amino acid residues of said CMV polypeptide corresponding to amino acid residues of position 84 and position 85 of SEQ ID NO:62; and
(iii) a T helper cell epitope, wherein said T helper cell epitope replaces a N-terminal region of said CMV polypeptide.

2. The modified VLP of CMV of claim 1, wherein said chimeric CMV polypeptide further comprises a first amino acid linker, wherein said first amino acid linker is positioned at the N- or at the C-terminus of said antigenic polypeptide, and wherein preferably said first amino acid linker has a length of at most 30 amino acids.

3. The modified VLP of CMV of claim 2, wherein said chimeric CMV polypeptide further comprises a second amino acid linker, wherein said first amino acid linker is positioned at the N-terminus of said antigenic polypeptide, and said second amino acid linker is positioned at the C-terminus of said antigenic polypeptide, and wherein preferably said second amino acid linker has a length of at most 30 amino acids.

4. The modified VLP of CMV of claim 2 or claim 3, wherein said first and said second amino acid linker is independently selected from the group consisting of:
(a.) a polyglycine linker (Gly)ₙ of a length of n=2-10;
(b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and
(c.) an amino acid linker comprising at least one Gly, at least one Ser, and at least one amino acid selected from Thr, Ala, Lys, Asp and Glu.

5. The modified VLP of CMV of claim 2 or claim 3, wherein said first and/or said second amino acid linker is independently selected from (i) a glycine-serine linker (GS-linker) comprising at least one glycine and at least one serine, wherein said GS linker has an amino acid sequence of (GS)ᵣ(GₛS)ₜ(GS)ᵤ with r=0 or 1, s=1-5, t=1-5 and u=0 or 1, and (ii) a glycine-serine-glutamic acid-aspartic acid linker (GSED-linker) comprising at least one glycine, at least one serine, at least one glutamic acid and at least one aspartic acid, wherein said GSED linker comprises an amino acid sequence of (DED)ₓ(GₛS)ₜ(G)_{y}(DED)_{z}(GS)ᵤ with s=1-5, t=1-5, u=0 or 1, x=0 or 1, y=0-5 and z=0 or 1.

6. The modified VLP of CMV of any one of the preceding claims, wherein said CMV polypeptide is a coat protein of CMV or an amino acid sequence having a sequence identity of at least 90%, preferably 95% with SEQ ID NO:62.

7. The modified VLP of CMV of any one of the preceding claims, wherein said CMV polypeptide comprises, or preferably consists of,
(i) an amino acid sequence of a coat protein of CMV, wherein said amino acid sequence comprises, or preferably consists of, SEQ ID NO:62; or
(ii) an amino acid sequence having a sequence identity of at least 90% of SEQ ID NO:62; and
wherein said amino sequence as defined in (i) or (ii) comprises SEQ ID NO:63 or an amino acid sequence region, wherein said amino acid sequence region has a sequence identity of at least 90% with SEQ ID NO:63.

8. The modified VLP of CMV of any one of the preceding claims, wherein said N-terminal region of said CMV polypeptide corresponds to amino acids 2-12 of SEQ ID NO:62.

9. The modified VLP of CMV of any one of the preceding claims, wherein said Th cell epitope is derived from tetanus toxin or is a PADRE sequence.

10. The modified VLP of CMV of any one of the preceding claims, wherein said Th cell epitope comprises the amino acid sequence of SEQ ID NO:64 or SEQ ID NO:65.

11. The modified VLP of CMV of any one of the preceding claims, wherein said chimeric CMV polypeptide comprises the amino acid sequence of SEQ ID NO:5 or SEQ ID NO:66, wherein said antigenic polypeptide is inserted into said chimeric CMV polypeptide of SEQ ID NO:5 between amino acid residues of position 88 and position 89 of SEQ ID NO:5 or wherein said antigenic polypeptide is inserted into said chimeric CMV polypeptide of SEQ ID NO:66 between amino acid residues of position 86 and position 87 of SEQ ID NO:66.

12. The modified VLP of CMV of any one of the preceding claims, wherein said modified VLP of CMV further comprises at least one CMV protein, wherein said CMV protein comprises a coat protein of CMV or an amino acid sequence having a sequence identity of at least 75%, preferably of at least 85% with SEQ ID NO:62, and wherein said CMV protein is optionally modified by a T helper cell epitope, and wherein preferably said coat protein of CMV comprises SEQ ID NO:62.

13. The modified VLP of CMV of any one of the preceding claims, wherein said antigenic polypeptide is a polypeptide derived from the group consisting of: (a) allergens; (b) viruses; (b) bacteria; (c) parasites; (d) tumors; (e) self-molecules; (h) hormones; (i) cytokines; and (k) chemokines.

14. The modified VLP of CMV of any one of the preceding claims, wherein said antigenic polypeptide is an allergen, a self antigen, a tumor antigen, or a polypeptide of a pathogen.

15. The modified VLP of CMV of any one of the preceding claims, wherein said chimeric CMV polypeptide is selected from the group consisting of SEQ ID NO:6, SEQ ID NO:7, SEQ ID NO:8, SEQ ID NO:9, SEQ ID NO:29, SEQ ID NO:39, SEQ ID NO:46, SEQ ID NO:52, SEQ ID NO:53, SEQ ID NO:54, SEQ ID NO:128, SEQ ID NO:132, SEQ ID NO:134 or SEQ ID NO:139.
